(19) United States
(12) Patent Application Publication
(10) Pub. No.: US 2025/0352640 A1
(43) Pub. Date: Nov. 20, 2025

(54) MULTI-EPITOPE CONSTRUCT

(71) Applicants: eTheRNA immunotherapies NV, Niel (BE); myNEO, Gent (BE)

(72) Inventors: Filipe BRANCO MADEIRA, Niel (BE); Cedric BOGAERT, Gent (BE); Lena PFITZER, Gent (BE); Bruno FANT, Gent (BE)

(21) Appl. No.: 18/862,813

(22) PCT Filed: May 5, 2023
(86) PCT No.: PCT/EP2023/061894

§ 371 Date: Nov. 4, 2024

(30) Foreign Application Priority Data
May 5, 2022 (EP) 22171827.3

(51) Int. Cl. A61K 39/215; A61K 39/00; A61P 31/14; C07K 14/165; C12N 15/11

(52) U.S. Cl. CPC A61K 39/215; A61P 31/14; C07K 14/165; C12N 15/11; A61K 2039/53; A61K 2039/70

(57) ABSTRACT

The invention is situated in the field of vaccination therapy. More specifically, the invention relates to a multi-epitope construct comprising nucleic acid sequences encoding peptides or functional variants and fragments thereof derived from a coronavirus. The invention further relates to a combination, polypeptides, or pharmaceutical composition for use in the treatment or prevention a coronavirus in a subject; in particular the SARS-COV-2 virus.

### FIELD OF THE INVENTION

The invention is situated in the field of vaccination therapy. More specifically, the invention relates to a multi-epitope construct comprising nucleic acid sequences encoding peptides or functional variants and fragments thereof derived from a coronavirus. The invention further relates to a combination, polypeptides, or pharmaceutical composition for use in the treatment or prevention a coronavirus in a subject; in particular the SARS-COV-2 virus.

### BACKGROUND OF THE INVENTION

The emergence of the COVID-19 pandemic is a threat to the human population worldwide as the infection is highly transmissible and causes severe disease and mortality. Up until now, the global health burden and economy remains hypercritical. Currently, there are three main types of COVD-19 vaccines (mRNA vaccines, viral vector vaccines, and protein subunit vaccines), of which the mRNA and vector vaccines have been successfully used in global vaccination strategies. mRNA vaccines (Moderna; WO2021154763A1 and Pfizer-BioNTech; WO2021188969A2) use genetic information from SARS-COV-2, the virus that causes COVID-19 and gives our cells instructions how to make a harmless protein that is unique to the virus. In a viral vector vaccine (Johnson, AstraZeneca, Sputnik V, Convidecia) genetic information from SARS-COV-2 is placed in a modified version of a different virus. Protein subunit vaccines (Novavax) include harmless pieces (proteins) of SARS-COV-2 instead of the entire virus.

mRNA vaccines represent a promising alternative to conventional vaccine approaches because of their ease of design, capacity for rapid development, potential for low-cost manufacture, safe administration, the induction of both cellular and humoral immunity, and lack of interaction with the genomic DNA. Recent improvements in mRNA vaccines act to increase protein translation, modulate innate and adaptive immunogenicity and improve delivery. While some messenger RNA vaccines, such as the Pfizer-BioNTech COVID-19 vaccine, have the disadvantage of requiring ultra-cold storage before distribution, other mRNA vaccines do not have such requirements.

SARS-COV-2 is a member of the beta corona virus genus, causing pneumonia. SARS-COV-2 is an enveloped virus with single stranded RNA, belonging to the corona viridae family and can cause infection in mammals, birds and humans. The whole genome of SARS-COV-2 was sequenced (Wu et al., 2020) and is approximately 29.9 kb. The availability of the genome had opened the opportunity to develop vaccine against this devastating disease. For COVID-19 vaccines, all the approved mRNA vaccines so far use a spike protein-based approach (Moderna; WO2021154763A1 and Pfizer-BioNTech; WO2021188969A2). A vaccine based on the spike protein induces antibodies that block SARS-COV-2 binding to host cell receptors and therefore neutralize virus infection leading to potent protective immunity. A concerning feature of the spike protein of SARS-COV-2 is how it changes over time during the evolution of the virus. As with many viruses, the viral genome of SARS-COV-2 can mutate and numerous such new variants have been described during the pandemic for SARS-COV-2. These mutations can change the biochemical properties of the spike protein and therefore evade or escape the neutralization capacity of neutralizing antibodies generated against previous variants as the virus evolves. This therefore provides opportunities to search for improved vaccine designs that avoid or reduce the impact if such new “variants of concern” (VOC).

It is therefore the object of the current invention to design a multi-epitope based vaccine comprising of T-cell epitopes based on structural and non-structural proteins other than the spike protein derived from a coronavirus to provide broad protective and durable immunity to multiple coronavirus variants and even strains (pan-corona virus vaccine). Optionally, this multi-epitope vaccine can be combined with one or more constructs encoding full length spike variants providing neutralizing antibody responses similar to the established COVID-19 vaccines mentioned above.

### SUMMARY OF THE INVENTION

In a first aspect, the present invention relates to a multi-epitope construct comprising at least five nucleic acid sequences encoding peptides or functional variants and/or fragments thereof derived from a coronavirus wherein said peptides, variants and/or fragments thereof comprise amino acid sequences having at least 95% sequence identity to the sequences selected from the list comprising SEQ ID NO: 1-47.

In a following embodiment of the present invention, the nucleic acid sequences are optimized with codon optimization.

In a specific embodiment, the present invention provides a multi-epitope construct as defined herein, further comprising one or more nucleic acid sequences encoding a coronaviral glycoprotein or functional variants and fragments thereof, in particular a SARS-COV-2 spike glycoprotein.

In a specific embodiment, the present invention provides a combination comprising said multi-epitope construct and a construct comprising one or more nucleic acid sequences encoding a coronaviral glycoprotein or functional variants and fragments thereof, in particular a SARS-COV-2 spike glycoprotein.

In yet a further embodiment, the present invention provides said multi-epitope construct, or said combination, wherein the encoded peptides or functional variants and fragments thereof are separated with at least one specific molecular linker selected from the list comprising: a flexible linker, a rigid linker, and/or a cleavable linker.

In a further aspect, the present invention provides a multi-epitope construct of the invention wherein said construct comprises at least five nucleic acid sequences having at least 95% sequence identity to the sequences as set forth in SEQ ID NO: 48-94; in particular RNA molecules.

In a further aspect, the present invention provides a multi-epitope construct as defined herein, wherein said at least 5 nucleic acid sequences are selected from any one of the following lists:
a. SEQ ID NO: 55, 89, 57, 53, 49, 92, 70, 71, 48, 74, 79, 77, 87, 65, 59, 88, 72, 60, 81;
b. SEQ ID NOs: 55, 65, 59, 76, 83, 60, 94, 71, 49, 74, 79, 87, 88, 89, 48, 58, 68, 92, 51, 52, 57, 85, 81, 53, 67, 72, 70;
c. 55, 65, 59, 76, 83, 60, 94, 71, 49, 74, 79, 87, 88;
d. SEQ ID NOs: 89, 48, 58, 68, 92, 51, 52, 57, 85, 81, 53, 67, 72, 70;
e. SEQ ID NOs: 60, 63, 54, 49, 84, 89, 71, 57, 65, 91, 48, 56, 66, 80, 79, 67, 78, 75, 59, 82;
f. SEQ ID NOs: 91, 71, 78, 66, 86, 63, 60, 80, 90, 75, 73, 54, 67, 61, 89, 48, 65, 79, 62, 57, 50, 93, 84, 49, 59, 64, 69, 82, 56;
g. SEQ ID NOs: 91, 71, 78, 66, 86, 63, 60, 80, 90, 75, 73, 54, 67, 61;

In yet another aspect, the present invention provides a polypeptide encoded by said multi-epitope construct.

In a particular embodiment, the present invention provides a pharmaceutical composition comprising said multi-epitope construct, said combination, or said polypeptide, and at least one pharmaceutically acceptable agent.

In another embodiment, the present invention provides said multi-epitope construct, said combination, said polypeptide or said pharmaceutical composition; which is formulated in liposomes or nanoparticles, such as lipid nanoparticles or polymeric nanoparticles; in particular lipid nanoparticles.

In a further aspect, the present invention provides said multi-epitope construct, said combination, said polypeptide or said pharmaceutical composition for use in human or veterinary medicine.

In yet embodiment, the present invention provides said multi-epitope construct, said combination, said polypeptide or said pharmaceutical composition for use in vaccination, in particular intramuscular vaccination.

In a specific embodiment, the present invention provides said multi-epitope construct, said combination, said polypeptide or said pharmaceutical composition for use of inducing an immune response against a coronavirus in a subject; in particular the SARS-COV-2 virus.

In yet another specific embodiment, the present invention provides said multi-epitope construct, said combination, said polypeptide or said pharmaceutical composition for use in the treatment or prevention a coronavirus in a subject; in particular the SARS-COV-2 virus.

In a further aspect, the present invention relates to a method of inducing an immune response against a coronavirus, comprising: administering a therapeutically effective amount of said multi-epitope construct, said combination, said polypeptide or said pharmaceutical composition to a subject.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Immunogenicity of mRNA constructs encoded SARS-COV-2 epitopes in mouse. Female BALB/c mice (6-8 weeks old) were injected intramuscularly (on day 0 and day 21) with mRNA vaccine compositions at dose of 5 μg. As a negative control, one group of mice is vaccinated with buffer (TBS). Vaccination with mRNA encoding concatenated epitopes derived from conserved regions of the non-structural proteins of SARS-COV-2 induced strong epitope-specific CD8 T cell responses after a prime/boost vaccination with mRNA-LNPs.

FIG. 2: Antigen (SARS-COV-2 Spike Delta) specific serum Immunoglobulin (IgG1 and IgG2a) titers after prime (21 days) and boost (35 days) intramuscular vaccination of mice with a dose-range amounts of LNP formulated mRNA vaccine. The dose indicated (0.2 to 10 μg) corresponds to the amount of Spike-Delta mRNA delivered to a single mouse at each injection event and is half of the total amount of mRNA as this is a combination containing the same amount of construct2.3 mRNA. There is a clear effect of the vaccine as seen in relatively high IgG titers, a very visible boosting effect from d21 to d35 and a plateau effect above 1 μg of mRNA. The empty LNP group included does not contain any mRNA in the formulation and was prepared based on calculations of the other groups to contain the same amount of lipids as the higher dose

FIG. 3: CD8+ multifunctional T cell responses after restimulation with a peptide library corresponding to the vaccination antigen (SARS-COV-2 Spike, Delta). Splenocytes were collected from the same groups (animals vaccinated 2 times with an LNP formulated mRNA vaccine against SARS-COV-2 consisting of equal amounts of Spike Delta mRNA and construct2.3 at different doses), at day 35 (terminal), and restimulated with a mix of peptides scanning the whole Spike protein and analyzed by flow cytometry. We see a clear dose-range response for CD8+ T cells expressing IFNg (and CD107a) after restimulation.

FIG. 4: Concatenated sequence of SARS-COV-2 construct 2.1_DCL (SEQ ID NO: 95).

FIG. 5: Concatenated sequence of SARS-COV-2 construct 2.2_DCL (SEQ ID NO: 96).

FIG. 6: Concatenated sequence of SARS-COV-2 construct 2.2.2_DCL (SEQ ID NO: 98)

FIG. 7: Concatenated sequence of SARS-COV-2 construct 2.3_DCL (SEQ ID NO: 99)

FIG. 8: Concatenated sequence of SARS-COV-2 construct 2.4_DCL (SEQ ID NO: 100)

FIG. 9: Concatenated sequence of SARS-COV-2 construct 2.4.1_DCL (SEQ ID NO: 101)

FIG. 10: Concatenated sequence of SARS-COV-2 construct 2.4.2_DCL (SEQ ID NO: 102)

FIG. 11: Concatenated sequence of SARS-COV-2 construct 2.5_DCL (SEQ ID NO: 103)

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The terms “comprising”, “comprises” and “comprised of” as used herein are synonymous with “including”, “includes” or “containing”, “contains”, and are inclusive or open-ended and do not exclude additional, non-recited members, elements, or method steps. The terms also encompass “consisting of” and “consisting essentially of”.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

In search for new pan-corona virus vaccines, the inventors investigated whether a nucleic acid vaccine based on expression of other proteins than the spike glycoprotein derived from a coronavirus can be used to induce a broad protective and durable immunity to multiple coronavirus strains. By using bioinformatics tools, the inventors selected a set of conserved peptide windows for further epitope prediction. The inventors unexpectedly found a set of conserved peptide windows that are potential antigenic determinants that evoke the production of specific T cells and thus a strong antiviral immune response in the host. As a results, the inventors designed a multi-epitope construct comprising nucleic acid sequences encoding for at least 5 peptide windows or functional variants and fragments thereof derived from a coronavirus for use in the treatment or prevention of a coronavirus infection in a subject; in particular the SARS-COV-2 virus. The major advantage associated with the multi-epitope vaccine is the induction of a broad immune response against different viral proteins reducing the risk of immune escape during virus evolution.

In a first aspect, the present invention relates to a multi-epitope construct comprising at least five nucleic acid sequences encoding peptides or functional variants and/or fragments thereof derived from a coronavirus wherein said peptides, variants and/or fragments thereof comprise amino acid sequences having at least 95% sequence identity to the sequences selected from the list comprising SEQ ID NO: 1-47.

In the context of the present invention, the term “construct” refers to an artificially-designed segment of nucleic acids that can be used to incorporate genetic material into a target tissue or cell. As used herein, the multi-epitope construct is delivered as a transcript of interest in the host cell cytoplasm where expression generates translated protein(s) to be located within the membrane, secreted or intracellularly located. It should be understood that the translated proteins can be one or more immunogen(s) derived from a coronavirus.

In some embodiments, the construct defined in the present invention can be a multi-epitope DNA construct or a multi-epitope RNA construct, in particular a multi-epitope messenger RNA (mRNA) construct.

In the context of the present invention, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. In particular, the term refers to single stranded RNA, but may also refer to double stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

According to the present invention, the term “RNA” includes and preferably relates to “mRNA” which means “messenger RNA” and relates to a “transcript” which may be produced using DNA as template and encodes a peptide or protein. mRNA typically comprises a 5′ untranslated region (5′-UTR), a protein or peptide coding region and a 3′ untranslated region (3′-UTR). The RNA further comprises a 3′ poly(A) tail and/or a 5′ cap analog. mRNA has a limited halftime in cells and in vitro. The skilled artisan will appreciate that, except where otherwise noted, nucleic acid sequences set forth in the instant application may recite “U”s in a representative RNA sequence but where the sequence represents DNA, the “U”s would be substituted for “T”s. Thus, any of the RNA sequences disclosed herein and identified by a particular sequence identification number herein, is also intended to disclose its corresponding DNA sequence complementary to the RNA, where each “U” of the RNA sequence is substituted with “T”.

For the sake of clarity, an mRNA molecule encompasses any coding RNA molecule, which may be translated by a eukaryotic host into a protein.

In some embodiments, the RNA can be non-replicating RNA (NRM), also termed non-amplifying RNA. Non-amplifying mRNA has only one open reading frame which codes for the antigen protein of interest. The total amount of mRNA used by the cell is equal to the amount of mRNA delivered by the vaccine and thus, dosage strength is limited to the delivered amount of RNA.

In some embodiments, the RNA can be self-amplifying RNA (SAM). SAM has two open reading frames. The first open reading frame, like conventional mRNA, codes for the antigen protein of interest. The second open reading frame codes for an RNA-dependent RNA polymerase (and its helper proteins) which self-replicates the mRNA construct in the cell and creates multiple self-copies.

In some embodiments, the RNA can be a linear or a circular RNA, preferably linear, more preferably a linear non-amplifying RNA.

The term ‘modified mRNA molecules’ means mRNA molecules that contain one or more modified nucleosides (termed “modified nucleic acids”), which have useful properties such as the lack of a substantial induction of the innate immune response of a cell into which the mRNA is introduced. These modified nucleic acids enhance the efficiency of protein production, intracellular retention of nucleic acids, and viability of contacted cells, as well as possess reduced immunogenicity.

In some embodiments, modified nucleobases in nucleic acids (e.g. RNA nucleic acids, such as mRNA nucleic acids) comprise 1-methyl-pseudouridine (m 1 y), 1-ethyl-pseudouridine (e|\|/), 5-methoxy-uridine (mo5U), 5-methyl-cytidine (m5C), and/or pseudouridine (y). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

Preferably, mRNA is produced by in vitro transcription using a DNA template. In one embodiment of the invention, the RNA is obtained by in vitro transcription. The in vitro transcription methodology is known to the skilled person and may comprise a purified linear DNA template containing a promoter, ribonucleotide triphosphates, a buffer system that includes dithiothreitol (DTT) and magnesium ions, spermidine and an appropriate RNA polymerase such as T7 RNA polymerase. The exact conditions used in the transcription reaction depend on the amount of RNA needed for a specific application. There is a variety of in vitro transcription kits commercially available.

In another embodiment, the present invention also provides a multi-epitope DNA construct comprising at least five nucleic acid sequences encoding peptides or functional variants and fragments thereof derived from a coronavirus wherein said peptides, variants and/or fragments thereof comprise amino acid sequences having at least 95% sequence identity to the sequences selected from the list comprising SEQ ID NO: 1-47; wherein each U of the RNA sequence is replaced with a T in the corresponding DNA sequence.

In the context of the present invention, the term “DNA construct” is meant to be a type of construct that works by injecting genetically engineered plasmid containing the DNA sequence encoding the antigen(s) which is then taken up by host cells and transcribed and translated into the encoded genes of interest against which an immune response is sought, so the cells directly produce the antigen, thus causing a protective immunological response. This approach offers a number of potential advantages over traditional approaches, including the stimulation of both B- and T-cell responses, improved vaccine stability, the absence of any infectious agent and the relative ease of large-scale manufacture.

As recognized by those skilled in the art, types of DNA constructs may include but are not limited to bacterial plasmids, bacteriophage vectors, artificial chromosomes or fosmids.

In the context of the present invention, the term “epitope” refers to an antigenic determinant (e.g. a polypeptide of a coronavirus) capable of inducing an immune response, in particular a cellular or a humoral immune response. An immune response is to be understood as the ability of an immune system to produce cytotoxic and helper T cells recognizing the antigens presented by infected host cells or antibodies against antigens. The epitope, which is a small site on an antigen, interacts with a specific antigen-binding site of an antigen-binding protein, e.g., a variable region of an molecule cell receptor. Epitopes may be defined as structural or functional. Functional epitopes are generally a subset of the structural epitopes and have those residues that directly contribute to the affinity of the interaction. Epitopes may be linear or conformational, that is, composed of non-linear amino acids. A single antigen may have more than one epitope. As used herein, the term “multi-epitope” refers to a series of potentially overlapping peptides which allow the induction of a broad immune response. The series of peptides might harbor T-cell and B-cell epitopes.

The term “epitope” also refers to a site on an antigen to which T and/or B cells respond. As used herein, it should be understood that the term “epitope” or “antigen” encompasses immunogenic proteins and immunogenic fragments, unless otherwise stated. “Viral antigens” refer to antigens encoded by a virus. They include, but are not limited to, antigens of coronaviruses, such as COVID-19.

The multi-epitope construct of the present invention comprises nucleic acid molecules encoding various sites of coronavirus antigens. In the context of the present invention, the term “coronavirus antigen” should be understood as an arrangement of amino acids that make part of the gene products encoded by a given coronavirus. The genome organization of a coronavirus is as follows: 5′-leader-UTR-ORF1a-ORF1b-spike(S) gene-envelope (E) gene-membrane (M) gene-nucleocapsid (N) gene-3-UTR-poly (A) tail. The ORF1a and ORF1b encode the replicase polyprotein which itself cleaves to form 16 nonstructural proteins (nsp1-nsp16). The ORF1a encodes for nsp1-nsp10, while ORF1b encodes for nsp11-nsp16. The accessory genes are distributed in between the structural genes and the number of accessory proteins and their function is unique depending on the specific coronavirus (e.g. for the SARS-COV-2 virus, the following accessory genes are present: ORF3a, ORF 6, ORF7a, ORF7b, ORF8a, ORF9b and ORF10).

In the context of the present invention, the multi-epitope construct may comprise at least two nucleic acid sequences, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 encoding for coronavirus peptides or functional variants and fragments thereof. In a particular embodiment, the multi-epitope construct comprises at least two nucleic acid sequences encoding for coronavirus peptides or fragments thereof. In a preferred embodiment, the multi-epitope construct comprises at least five nucleic acid sequences encoding for coronavirus peptides or fragments thereof.

In one embodiment, the multi-epitope construct of the present disclosure may comprise nucleic acid sequences encoding coronavirus peptides or functional variants and fragments thereof selected from the list comprising: E gene, M gene, N gene, ORF3a, ORF6, ORF7a, ORF7b, ORF8a, ORF9b and ORF10, ORF1a and/or ORF1b viral genomic window.

In a particular embodiment, the ORF1a and ORF1b coronavirus peptides or functional variants and fragments may be selected from the list comprising: NSP1, NSP2, NSP3, NSP4, NSP5, NSP6, NSP7, NSP8, NSP9, NSP10, NSP11, NSP12, NSP13, NSP14, NSP15, NSP16, or a combination thereof.

In a further embodiment, the multi-epitope construct of the present disclosure may comprise nucleic acid sequences encoding coronavirus peptides or functional variants and fragments thereof selected from the list comprising: E, M, N, NSP1, NSP3, NSP4, NSP5, NSP6, NSP8, NSP9, NSP12, NSP13, NSP14, NSP15, NSP16, ORF6 or a combination thereof.

In one embodiment, the SEQ ID numbers for the amino acid sequence for coronavirus antigens, as well as the SEQ ID numbers for the nucleic acid sequences encoding them are listed in respectively Table 1 and Table 2.

It should be understood that the amino acid sequences described herein are not limitative and can encompass sequence variation (i.e. have a percentage sequence identity to the described sequence).

In determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp. Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Hence, in one embodiment, a sequence having a given percentage sequence identity as given herein before is a sequence having one, two, three or more conservative amino acid substitutions as compared to the reference sequence.

On the other hand, variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

Hence, in a particular embodiment, the amino acid sequences described herein are at least and/or about 90%, 91%, 92%, 93%, 94%, 95%, 96%, 97%, 98%, 99% or 100% identical to the SEQ IDs recited in table 1.

In one embodiment, the present invention relates to a multi-epitope construct comprising at least two such as at least five nucleic acid sequences encoding peptides or functional variants and fragments thereof derived from a coronavirus wherein said peptides, variants and/or fragments thereof comprise amino acid sequences selected from the list comprising SEQ ID NO: 1-47, or having at least 90% such as at least 95% sequence identity thereto.

In a particular embodiment, the present invention relates to a multi-epitope construct comprising at least two such as at least five nucleic acid sequences encoding peptides or functional variants and fragments thereof derived from a coronavirus comprising amino acid sequences selected from the list comprising SEQ ID NO: 1, 2, 6, 8, 10, 12, 13, 18, 23-25, 27, 30, 32, 34, 40-42, 45, or having at least 90% such as at least 95% sequence identity thereto.

In a further embodiment, the present invention relates to a multi-epitope construct comprising at least two such as at least five nucleic acid sequences encoding peptides or functional variants and fragments thereof derived from a coronavirus comprising amino acid sequences selected from the list comprising SEQ ID NO: 1, 2, 4, 5, 6, 8, 10-13, 18, 20, 21, 23-25, 27, 29, 32, 34, 36, 38, 40-42, 45, 47, or having at least 90% such as at least 95% sequence identity thereto.

In another embodiment, the present invention relates to a multi-epitope construct comprising at least two such as at least five nucleic acid sequences encoding peptides or functional variants and fragments thereof derived from a coronavirus comprising amino acid sequences selected from the list comprising SEQ ID NO: 1, 2, 7, 9, 10, 12, 13, 16, 18-20, 24, 28, 31-33, 35, 37, 42, 44, or having at least 90% such as at least 95% sequence identity thereto.

In a specific embodiment, the present invention relates to a multi-epitope construct comprising at least two such as at least five nucleic acid sequences encoding peptides or functional variants and fragments thereof derived from a coronavirus comprising amino acid sequences selected from the list comprising SEQ ID NO: 1, 2, 3, 7, 9, 10, 12-20, 22, 24, 26, 28, 31-33, 35, 37, 39, 42-44, 46, or having at least 90% such as at least 95% sequence identity thereto.

In a particular embodiment, the present invention relates to a multi-epitope construct comprising at least two such as at least five nucleic acid sequences encoding peptides or functional variants and fragments thereof derived from a coronavirus comprising amino acid sequences selected from the list comprising SEQ ID NO: 1, 2, 10, 12, 13, 18, 20, 24, 32, 42, or having at least 90% such as at least 95% sequence identity thereto.

In Table 3, examples are listed of multi-epitope constructs with corresponding nucleic acid sequences (SEQ ID NO: 95-103) encoding amino acid sequence for coronavirus antigens.

In a particular embodiment, the present invention relates to a multi-epitope construct according to SEQ ID NO: 95 (construct 2.1) comprised of nucleic acid sequences: SEQ ID NO: 55, 89, 57, 53, 49, 92, 70, 71, 48, 74, 79, 77, 87, 65, 59, 88, 72, 60, 81, or having at least 90% such as at least 95% sequence identity thereto.

In a particular embodiment, the present invention relates to a multi-epitope construct according to SEQ ID NO: 96 (construct 2) comprised of nucleic acid sequences: SEQ ID NO: 55, 65, 59, 76, 83, 60, 94, 71, 49, 74, 79, 87, 88, 89, 48, 58, 68, 92, 51, 52, 57, 85, 81, 53, 67, 72, 70, or having at least 90% such as at least 95% sequence identity thereto.

In a particular embodiment, the present invention relates to a multi-epitope construct according to SEQ ID NO: 97 (construct 2.2.1) comprised of nucleic acid sequences: SEQ ID NO: 55, 65, 59, 76, 83, 60, 94, 71, 49, 74, 79, 87, 88, or having at least 90% such as at least 95% sequence identity thereto.

In a particular embodiment, the present invention relates to a multi-epitope construct according to SEQ ID NO: 98 (construct 2.2.2) comprised of nucleic acid sequences: SEQ ID NO: 89, 48, 58, 68, 92, 51, 52, 57, 85, 81, 53, 67, 72, 70, or having at least 90% such as at least 95% sequence identity thereto.

In a particular embodiment, the present invention relates to a multi-epitope construct according to SEQ ID NO: 99 (construct 2.3) comprised of nucleic acid sequences: SEQ ID NO: 60, 63, 54, 49, 84, 89, 71, 57, 65, 91, 48, 56, 66, 80, 79, 67, 78, 75, 59, 82, or having at least 90% such as at least 95% sequence identity thereto.

In a particular embodiment, the present invention relates to a multi-epitope construct according to SEQ ID NO: 100 (construct 2.4) comprised of nucleic acid sequences: SEQ ID NO: 91, 71, 78, 66, 86, 63, 60, 80, 90, 75, 73, 54, 67, 61, 89, 48, 65, 79, 62, 57, 50, 93, 84, 49, 59, 64, 69, 82, 56, or having at least 90% such as at least 95% sequence identity thereto

In a particular embodiment, the present invention relates to a multi-epitope construct according to SEQ ID NO: 101 (construct 2.4.1) comprised of nucleic acid sequences: SEQ ID NO: 91, 71, 78, 66, 86, 63, 60, 80, 90, 75, 73, 54, 67, 61, or having at least 90% such as at least 95% sequence identity thereto.

In a particular embodiment, the present invention relates to a multi-epitope construct according to SEQ ID NO: 102 (construct 2.4.2) comprised of nucleic acid sequences: SEQ ID NO: 89, 48, 65, 79, 62, 57, 50, 93, 84, 49, 59, 64, 69, 82, 56, or having at least 90% such as at least 95% sequence identity thereto.

In a particular embodiment, the present invention relates to a multi-epitope construct according to SEQ ID NO: 103 (construct 2.5) comprised of nucleic acid sequences: SEQ ID NO: 89, 59, 60, 49, 65, 79, 71, 67, 57, 48, or having at least 90% such as at least 95% sequence identity thereto.

In Table 4, examples of multi-epitope constructs with codon-optimized RNA sequences (three versions) are listed. It is therefore evident that the present invention also relates to multi-epitope constructs according to SEQ ID NOs: 104-130, or having at least 90% such as at least 95% sequence identity thereto.

For the sake of clarity, a multi-epitope construct may refer to one nucleic acid (such as mRNA) molecule comprising said at least 5 nucleic acid sequences encoding coronaviral epitopes, or it may refer to two or more nucleic acid (e.g. mRNA) molecules wherein each mRNA molecule comprises a specific set of the at least 5 selected nucleic acid sequences encoding coronaviral epitopes. In other words, lengthy constructs (e.g. >11 K b) are not always well expressed and therefore it might be preferred to encode the epitopes on two or more separate nucleic acid (e.g. mRNA) constructs (see also examples).

Within the context of the present invention, the term “coronavirus” encompasses all human coronaviruses (hCoV) such as but not limited to HCoV-OC43, HCoV-HKU1, HCOV-229E, HCoV-NL63, SARS-COV-2, SARS-COV, MERS-COV. It should be understood that peptides or fragments thereof encoded by the multi-epitope construct are common in in at least Sarbecoviruses including SARS-COV-2 and hence, induces an immune response against all these types of coronavirus. As used herein the multi-epitope construct can also be used in the context of a pan-coronavirus vaccine.

In a specific embodiment, the present invention provides a combination comprising said multi-epitope construct and a construct comprising one or more nucleic acid sequences encoding a coronaviral glycoprotein or functional variants and fragments thereof, in particular a SARS-COV-2 spike glycoprotein.

Alternatively, the present invention also provides a construct wherein sequences encoding for coronaviral glycoproteins are combined with sequences encoding peptides or functional variants and fragments thereof derived from a coronavirus comprising amino acid sequences selected from the list comprising SEQ ID NO: 1-47, or having at least 90% such as at least 95% sequence identity thereto;

Within the context of the present invention, the term “combination” refers to at least two separate multi-epitope constructs, of which one type of construct expresses peptides or fragments thereof as defined in Table 1, and at least one other type of multi-epitope construct expressing an additional protein and/or an adjuvant. It should be understood that the combination may comprise a plurality of different multi-epitope designs.

Examples of an additional protein can be an immunostimulatory protein, a co-stimulatory molecule, a cytokine, a chemokine, and/or an innate pathway triggering molecule. Adjuvants can be used for the protection of vaccine from degradation or to enhance or prolong vaccine immunogenicity.

In a specific embodiment, the combination refers to at least two separate constructs, of which one type of construct is a multi-epitope construct expressing peptides or fragments thereof as defined in Table 1, and at least one other construct expressing a coronal spike glycoprotein.

Within the context of the present invention, the term “spike glycoproteins(S)” is meant to be a specialized viral protein trimer acting as a chief mediator of attachment with the host cell receptors and viral entry. In one embodiment, the combination comprises nucleic acid sequences encoding a coronaviral glycoprotein or functional variants and fragments thereof selected from the list comprising HCoV-OC43, HCoV-HKU1, HCOV-229E, HCoV-NL63, MERS-COV, SARS-COV-2, SARS-COV, or a combination thereof, in particular a SARS-COV-2. It should be understood that S-proteins within the family of coronaviruses have an amino acid sequence similarity. For example, S-proteins of SARS-CoV and SARS-COV2 share approximately 76% identity in amino acid sequence. Hence, the combination described herein may comprise a construct comprising at least one nucleic acid molecule encoding a coronaviral glycoprotein (such as full length-S) or functional variants or at least fragments thereof, that covers all types of coronaviruses, or at least one type of coronavirus.

In some embodiments, the construct encoding a coronaviral glycoprotein comprises nucleic acid sequences that encode a coronavirus antigen variant. Antigen variants or other polypeptide variants refer to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

In Table 5, examples are listed of codon optimized RNA sequences of a construct (SEQ ID NO: 131-134) encoding a SARS-COV-2 spike glycoprotein.

In another particular embodiment, the combination comprises the multi-epitope construct of the invention in combination with a construct comprising one or more nucleic acid sequences encoding a SARS-COV-2 spike glycoprotein comprising an amino acid sequence as set forth in SEQ ID NOs: 131-134 or having at least 90% such as at least 95% sequence identity thereto.

In another specific embodiment, the combination refers to at least two separate constructs, of which one type is a multi-epitope construct expressing peptides or fragments thereof as defined in Table 1, and at least one other construct expressing at least 2 immunostimulatory proteins selected from the group comprising CD40L, CD70, and caTLR4. In a preferred embodiment, the at least one other multi-epitope construct comprises nucleic acid molecule encoding CD40L and CD70 (i.e. “DiMix”). In a more preferred embodiment, the at least one other construct can additionally comprise nucleic acid molecules encoding for the caTLR4, resulting in the so-called “TriMix”.

Throughout the invention, the term “TriMix” stands for a mixture of mRNA molecules encoding CD40L, CD70 and caTRLA4 immunostimulatory proteins.

In a further preferred embodiment, the specific combination of coronaviral glycoprotein and TriMix is used to improve the immunostimulatory effect of the construct encoding coronaviral epitopes as defined in Table 1.

In a specific embodiment, the combination of the present invention comprises at least two separate constructs, of which one type is a multi-epitope construct expressing peptides or fragments thereof as defined in Table 1, and at least one other construct expressing a plurality of predefined additional proteins, in particular a coronaviral glycoprotein or immunostimulatory proteins such as TriMix.

In a specific embodiment, the present invention may also provide a construct expressing peptides or fragments thereof as defined in Table 1, and expressing one or a plurality of predefined additional proteins, in particular a coronaviral glycoprotein or immunostimulatory proteins such as TriMix. In the context of the present invention, it should be understood that additional proteins and/or adjuvants can be encoded by the same additional construct; or be encoded by a plurality of separate constructs.

In yet a further embodiment, the present invention provides said multi-epitope construct, or said combination, wherein the encoded peptides are separated with at least one specific molecular linker selected from the list comprising: a flexible linker, a rigid linker, and/or a cleavable linker.

In yet another aspect, the present invention provides a polypeptide encoded by said multi-epitope construct.

In some embodiments, the multi-epitope construct encodes more than one polypeptide referred to as a fusion protein. In some embodiments, the construct further encodes a linker located between at least one or each domain of the fusion protein. Linkers play vital roles in splicing together epitopes and producing an extended conformation (flexibility), protein folding, and separation of functional domains, and therefore, make the protein structure more stable. Flexible and rigid linkers covalently join functional domains together to act as one molecule throughout the in vivo processes and thus are not cleaved. In some embodiments, the flexible linker is selected from the group comprising Gly, Ser, Thr, Lys, Glu, Thr, Ala or combinations thereof. In particular, an example can be stretches of Gly and Ser residues “(GSₙ” linker), wherein n indicates the length of this GS linker. An example of the most widely used flexible linker has the sequence of (Gly-Gly-Gly-Gly-Ser) n, GGS, GGSG, G. Other examples can be a GS linker comprising additional amino acids to improve solubility and flexibility. Several other types of flexible linkers include KESGSVSSEQLAQFRSLD (SEQ ID NO: 135) and EGKSSGSGSESKST (SEQ ID NO: 136). In some embodiments, the rigid linker can be for example an alpha helix-forming linkers with the sequence of (EAAAK)n, which is frequently applied to the construction of many recombinant fusion proteins. Another type of rigid linkers has a Pro-rich sequence, (XP)ₙ, with X designating any amino acid, preferably Ala, Lys, or Glu. An example of a rigid linker can be stretches of Glu and Pro residues “(GPₙ” linker), wherein n indicates the length of this GP linker, in particular GPG, GPPPG, GPGPG, GP8G, or PAPAP, PA.

On the other hand, the linker can be an in vivo cleavable linker, to release free functional domains in vivo. This type of linker may reduce steric hindrance, improve bioactivity, or achieve independent actions/metabolism of individual domains of recombinant fusion proteins after linker cleavage. For the sake of clarity, cleavable linkers known in the art may be used in connection with the disclosure. Exemplary such linkers include: F2A linkers, T2A linkers, P2A linkers, E2A linkers and combinations thereof (See, e.g., WO2017/127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

In some embodiments, the linker can be selected from the group comprising GGGGS, GGS, GGGS, GGSG, G, GS, Ankyrin repeat, EAAAK, GPG, GPPPG, GPGPG, GP8G, GTP, PAPAP, KK, AP, F2A, E2A, P2A, T2A, AAY and/or AYY. In some embodiments, the fusion protein contains three domains with intervening linkers, having the structure: domain-linker-domain-linker-domain.

In a further embodiment, the nucleic acid sequences encoding the peptides derived from a coronavirus are part of a single nucleic acid molecule. This single nucleic acid molecule is preferably capable of expressing several proteins independently. In a preferred embodiment, the nucleic acid sequences encoding the peptides derived from a coronavirus are linked in the single nucleic acid molecule by an internal ribosomal entry site (IRES), enabling separate translation of each of the two or more nucleic acid sequences into an amino acid sequence. Alternatively, a self-cleaving 2a peptide-encoding sequence is incorporated between the coding sequences of the different coronavirus antigens. In this way, two or more factors can be encoded by one single nucleic acid molecule.

The invention thus further provides for a multi-epitope construct comprising nucleic acid sequences encoding two or more peptides derived from a coronavirus, wherein the two or more coronavirus derived peptides are either translated separately from the single nucleic acid molecule through the use of an IRES between the two or more coding sequences. Alternatively, the invention provides an mRNA molecule encoding two or more coronavirus derived peptides separated by a self-cleaving 2a peptide-encoding sequence, enabling the cleavage of the two protein sequences after translation.

In a following embodiment of the present invention, the nucleic acid sequences are optimized with codon optimization.

Codon optimization methods are known in the art. For example, an ORF of any one or more of the sequences provided herein may be codon optimized. Codon optimization, in some embodiments, may be used to match codon frequencies in target and host organisms to increase GC content to increase mRNA stability or reduce secondary structures; minimize tandem repeat codons or base runs that may impair transcription or translation; customize transcriptional and translational control regions; insert or remove protein trafficking sequences; add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art. Codon-optimized mRNA sequences that are produced using different programs or approaches can vary dramatically because different codon optimization strategies differ in how they quantify codon usage and implement codon changes. Thus, mRNAs encoding the same polypeptide via different codon assignments may show variation in the amount of protein expressed. In the context of the present invention, acceptable variations in RNA sequences may exist between non-optimized and optimized RNA sequences. In some embodiments, the open reading frame (ORF) sequence is optimized using optimization algorithms.

In some embodiments, a codon optimized sequence shares less than about 99%, less than about 95%, less than about 90%, less than about 85%, less than about 80%, less than about 75%, less than about 70%, less than about 65%, sequence identity to a naturally-occurring or wild-type sequence ORF (e.g., a naturally-occurring or wild-type mRNA sequence encoding a coronavirus antigen). In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a coronavirus antigen). In some embodiments, a codon optimized sequence shares between 65% and 75% or about 80% sequence identity to a naturally-occurring or wild-type sequence (e.g., a naturally-occurring or wild-type mRNA sequence encoding a coronavirus antigen). In a specific embodiment, a codon optimized sequence shares 70% sequence identity to a naturally-occurring or wild-type sequence (e.g., a naturally-occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position). Alternatively, the degree of sequence identity between two or more nucleotide sequences may be calculated using a known computer algorithm for sequence alignment such as NCBI Blast v2.0, using standard settings.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings. A specific method utilizes the BLAST module of WU-BLAST-2 set to the default parameters, with overlap span and overlap fraction set to 1 and 0.125, respectively.

In a particular embodiment, the present invention provides a pharmaceutical composition comprising said multi-epitope construct, said combination, or said polypeptide, and at least one pharmaceutically acceptable agent.

As used herein, a “composition”, refers to any mixture of two or more products or compounds (e.g. agents, modulators, regulators, etc.). It can be a solution, a suspension, liquid, or aqueous formulations or any combination thereof.

In the context of the present invention, by means of the term “pharmaceutical composition” reference is made to a composition having pharmaceutical properties. In other words, reference is made to a composition providing for a pharmacological and/or physiological effect. Pharmaceutical compositions can comprise one or more pharmaceutically acceptable agents such as excipients, carriers, diluents.

In some embodiments, the pharmaceutically acceptable agents include, but are not limited to, biocompatible vehicles, adjuvants, additives, and diluents to achieve a composition usable as a dosage form. Additional suitable pharmaceutical carriers and diluents, as well as pharmaceutical necessities for their use, are described in Remington's Pharmaceutical Sciences.

As used herein and unless otherwise specified, the term “excipient” is to be understood as any substance formulated alongside the active compound included for the purpose of long-term stabilization such as prevention of denaturation or aggregation over the expected shelf life, bulking up liquid or solid formulations that contain potent active compound in small amounts (thus often referred to as “bulking agents”, “fillers”, or “diluents”), or to confer an enhancement on the active compound in the final dosage form, such as facilitating absorption, reducing viscosity, or enhancing solubility.

In another embodiment, the present invention provides said multi-epitope construct, said combination, said polypeptide or said pharmaceutical composition; which is formulated in liposomes or nanoparticles, such as lipid nanoparticles or polymeric nanoparticles; in particular lipid nanoparticles.

The constructs, combination, polypeptides, or pharmaceutical composition defined herein can be formulated in lipid nanoparticles (LNPs) that encapsulate the constructs to protect them from degradation and promote cellular uptake.

In the context of the present invention, by means of the term “lipid nanoparticle”, or LNP, reference is made to a nanosized particle composed of one or more lipids, e.g. a combination of different lipids. Possible lipids used in the LNP can be for example, but not limited to at least one phospholipids, at least one modified lipids such as PEG lipids, at least one ionisable lipids, at least one sterol. The lipid nanoparticles of the disclosure and the compositions thereof are generally known in the art.

In the context of the present invention, the term “PEG lipid” or alternatively “PEGylated lipid” is meant to be any suitable lipid modified with a PEG (polyethylene glycol) group. For example, PEG lipids in the context of the present invention can be C14-PEG lipids, such as for example DMG-PEG2000 (1,2-dimyristoyl-rac-glycero-3-methoxypolyethylene glycol-2000). C14-PEG lipids contain a polyethylene glycol moiety, which defines the molecular weight of the lipids, as well as a fatty acid tail comprising 14 C-atoms. Alternatively, the PEG lipids of the present invention may be C16- or C18-lipids.

In the context of the present invention the term “ionisable” (or alternatively cationic) in the context of a compound or lipid means the presence of any uncharged group in said compound or lipid which is capable of dissociating by yielding an ion (usually an H+ ion) and thus itself becoming positively charged. Alternatively, any uncharged group in said compound or lipid may yield an electron and thus becoming negatively charged. As used herein, any type of ionizable lipid can suitably be used. For example, suitable ionizable lipids are ionizable amino lipids which comprise 2 identical or different tails linked via an S—S bond.

In the context of the present invention, the term “phospholipid” is meant to be a lipid molecule consisting of two hydrophobic fatty acid “tails” and a hydrophilic “head” consisting of a phosphate groups. The two components are most often joined together by a glycerol molecule, hence, in the phospholipid of the present invention is preferably a glycerol-phospholipid. Furthermore, the phosphate group is often modified with simple organic molecules such as choline (i.e. rendering a phosphocholine) or ethanolamine (i.e. rendering a phosphoethanolamine).

In the context of the present invention, the term “sterol”, also known as steroid alcohol, is a subgroup of steroids that occur naturally in plants, animal and fungi, or can be produced by some bacteria. In the context of the present invention, any suitable sterol may be used, such as selected from the list comprising cholesterol, ergosterol, campesterol, oxysterol, antrosterol, desmosterol, nicasterol, sitosterol and stigmasterol; preferably cholesterol.

In a particular embodiment, said LNP comprises about and between 10 mol % and 60 mol % of said ionisable lipid; preferably about and between 40 mol % and 60 mol %.

In yet another specific embodiment, said LNP comprises about and between 15 mol % and 50 mol % of sterol; preferably about and between 20 mol % and 40 mol %.

In a further embodiment, said LNP comprises about and between 0.5 mol % and 10 mol % of said PEG lipid; preferably about and between 0.5 mol % and 5 mol %.

In another specific embodiment, said LNP comprises about and between 5 mol % and 40 mol % of said phospholipid; preferably about and between 5 mol % and 15 mol %.

Hence, in a more specific embodiment, the LNP of the present invention comprises about and between 10 mol % and 70 mol % of said ionisable lipid; and/or about and between 15 mol % and 50 mol % of sterol; and/or about and between 0.5 mol % and 10 mol % of said PEG lipid; and/or about and between 5 mol % and 40 mol % of said phospholipid.

In another specific embodiment, the LNP of the present invention comprises about and between 40 mol % and 60 mol % of said ionisable lipid; and about and between 20 mol % and 40 mol % of sterol; about and between 0.5 mol % and 5 mol % of said PEG lipid; and about and between 5 mol % and 15 mol % of said phospholipid.

In a particular embodiment, the LNP of the present invention comprises 50 mol % of ionizable lipid, 10 mol % of phospholipid, 1.5 mol % of PEG lipid and 38.5 mol % of sterol.

As used herein, the term “nanoparticle” refers to any particle having a diameter making the particle suitable for systemic, in particular intramuscular or intravenous administration, typically having a diameter of less than 1000 nanometers (nm), preferably less than 500 nm, even more preferably less than 200 nm, such as for example between 50 and 200 nm; preferably between 70 and 160 nm.

In some embodiments, the mixture of lipids forms lipid nanoparticles. In some embodiments, the constructs are formulated in the lipid nanoparticles. In some embodiments, the lipid nanoparticles are formed first as empty lipid nanoparticles and combined with the construct of the vaccine immediately prior to (e.g., within a couple of minutes to an hour of) administration.

To avoid any misunderstanding the LNP's of the present invention may comprise a single multi-epitope construct, or they may comprise multiple constructs, such as a combination of one or more constructs encoding immune modulating proteins and/or one or more construct encoding antigen-specific proteins.

In a very specific embodiment, said constructs encoding immunomodulatory molecules may be combined with one or more construct encoding peptides derived from a coronavirus. For example, the LNP's of the present invention may comprise constructs encoding peptides derived from a coronavirus; in combination with one or more constructs encoding the immunostimulatory molecules CD40L, CD70 and/or caTLR4 (such as Dimix or Trimix); further in combination with one or more constructs encoding a coronaviral glycoprotein.

Furthermore, it should be understood that the LNP's of the present invention may comprise said combination, said polypeptide or said pharmaceutical composition according to the present invention.

In some embodiments, two or more different constructs (e.g., mRNA) encoding antigens may be formulated in the same lipid nanoparticle.

In a further aspect, the present invention provides said multi-epitope construct, said combination, said polypeptide or said pharmaceutical composition for use in human or veterinary medicine.

Pharmaceutical compositions are particularly suitable as a vaccine.

In the context of the present invention, the term “vaccine” as used herein is meant to be any preparation intended to provide adaptive immunity (T cell responses and antibodies) against a disease. To that end, the term “vaccine” as meant herein comprises at least one multi-epitope construct, at least one combination, at least one polypeptide or at least one pharmaceutical composition, optionally formulated into an LNP, to which an adaptive immune response is mounted.

In some embodiments, said vaccine may comprises naked multi-epitope constructs, naked polypeptides which are suspended in a buffer solution.

Vaccines can be prophylactic (example: to prevent or ameliorate the effects of a future infection by any natural or “wild” pathogen), or therapeutic (example, to actively treat or reduce the symptoms of an ongoing disease). The administration of vaccines is called vaccination.

The present invention also provides a vaccine for use in human or veterinary medicine. The use of a vaccine is also intended. Finally, the invention provides a method for the prophylaxis and treatment of human and veterinary disorders, by administering a vaccine to a subject in need thereof.

In a specific embodiment, the present invention provides a vaccine for use in the treatment or prevention a coronavirus in a subject; in particular the SARS-COV-2 virus.

In the context of the present application, the terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptoms but has not yet been diagnosed as having it; (b) inhibiting the disease symptoms, i.e. arresting its development; or (c) relieving the disease symptom, i.e. causing regression of the disease or symptom.

In another specific embodiment, the present invention provides a vaccine for use of inducing an immune response against a coronavirus in a subject; in particular the SARS-COV-2 virus.

The term “immune response” used throughout the description is not intended to be limited to the types of immune responses that may have been exemplified herein. The term therefore encompasses all infectious agents to which vaccination would be beneficial to the subject.

The vaccine of the invention may be used for inducing an immune response, in particular an immune response against a disease-associated antigen or cells expressing a disease-associated antigen, such as an immune response against a coronaviral antigen. Preferably said immune response is a T cell response. In one embodiment, the disease-associated antigen is a coronaviral antigen. The antigen encoded by the construct comprised in the nanoparticles described herein preferably is a disease-associated antigen or elicits an immune response against a disease-associated antigen or cells expressing a disease-associated antigen.

In a further aspect, the present invention relates to a method of inducing an immune response against a coronavirus, comprising: administering a therapeutically effective amount of said vaccine to a subject.

Within the context of the present invention, the vaccine may be administered prophylactically or therapeutically as part of an active immunization scheme to healthy individuals or early in infection during the incubation phase or during active infection after onset of symptoms.

In some embodiments, the vaccine may be administered as a monotherapy or as a combination therapy with other coronaviral vaccines.

In some embodiments, the vaccine may be administered as a single dose, two doses, three doses, four doses, or repeated as applicable.

In some embodiments, the time of administration in the monotherapy may be, but is not limited to 1 week, 2 weeks, 3 weeks, 4 weeks, 2 months, 3 months, 6 months, 1 year and may be repeated every year.

In another embodiment, the time of administration between the injections in a combination therapy may be, but is not limited to 1 minute to 30 minutes, 30 minutes to 1 hours, 3 hours, 6 hours, 12 hours, 1 day, 1 week, 2 weeks, 3 weeks, 4 weeks, 2 months, 3 months, 6 months, 1 year.

In a particular embodiment, the subject is injected with a dose of about and between 10 to 100 microgram (μg). The effective amount of the construct, as provided herein, may be as low as 10 μg, administered for example as a single dose or as two 5 μg doses. In some embodiments, the effective amount is a total dose of 10 μg-100 μg. For example, the effective amount may be a total dose of 10 μg, 20 μg, 30 μg, 40 μg, 50 μg, 60 μg, 70 μg, 80 μg, 90 μg, 100 μg. In some embodiments, the effective amount is a total dose of 10 μg. In some embodiments, the effective amount is a total dose of 20 μg. In some embodiments, the effective amount is a total dose of 30 μg. In some embodiments, the effective amount is a total dose of 60 μg. In some embodiments, the effective amount is a total dose of 80 μg. In some embodiments, the effective amount is a total dose of 100 μg.

In yet another embodiment, the present invention provides said multi-epitope construct, said combination, said polypeptide or said pharmaceutical composition for use in vaccination, in particular intramuscular vaccination.

In some embodiments, the vaccine may be administered intramuscularly, subcutaneously, intranasally, intradermally, or through the lymphatic system, similarly to the administration of inactivated vaccines known in the art.

The vaccine of the present invention is in particular intended for intramuscular administration, i.e. the injection of liquid substance directly into a muscle.

The present invention also provides a vaccine; wherein the administered is intravenously, i.e. the infusion of liquid substance directly into a vein.

TABLE 1
Amino acid sequences of coronavirus derived peptides.

| SEQ ID | Peptide or fragment | Amino acid sequence |
|---|---|---|
| 1 | E_1-21 | MYSFVSEETGTLIVNSVLLFL |
| 2 | M_160-180 | DIKDLPKEITVATSRTLSYYK |
| 3 | M_37-57 | FAYANRNRFLYIIKLIFLWLL |
| 4 | M_8-28 | ITVEELKKLLEQWNLVIGFLF |
| 5 | N_105-125 | SPRWYFYYLGTGPEAGLPYGA |
| 6 | N_296-316 | TDYKHWPQIAQFAPSASAFFG |
| 7 | N_302-322 | PQIAQFAPSASAFFGMSRIGM |
| 8 | NS6(ORF6)_1-21 | MFHLVDFQVTIAEILLIIMRT |
| 9 | NSP1(ORF1a)_52-72 | GLVEVEKGVLPQLEQPYVFIK |
| 10 | NSP12(ORF1b)_114-134 | ISRQRLTKYTMADLVYALRHF |
| 11 | NSP12(ORF1b)_231-251 | VPVVDSYYSLLMPILTLTRAL |
| 12 | NSP12(ORF1b)_332-352 | KIFVDGVPFVVSTGYHFRELG |
| 13 | NSP12(ORF1b)_501-521 | SAGFPFNKWGKARLYYDSMSY |
| 14 | NSP12(ORF1b)_559-579 | GVSICSTMTNRQFHQKLLKSI |
| 15 | NSP12(ORF1b)_616-636 | GWDYPKCDRAMPNMLRIMASL |
| 16 | NSP12(ORF1b)_64-84 | NLIDSYFVVKRHTFSNYQHEE |
| 17 | NSP12(ORF1b)_680-700 | TSSGDATTAYANSVFNICQAV |
| 18 | NSP12(ORF1b)_852-872 | GTLMIERFVSLAIDAYPLTKH |
| 19 | NSP12(ORF1b)_886-906 | QYIRKLHDELTGHMLDMYSVM |
| 20 | NSP13(ORF1b)_131-151 | KLFAAETLKATEETFKLSYGI |
| 21 | NSP13(ORF1b)_228-248 | TSHTVMPLSAPTLVPQEHYVR |
| 22 | NSP13(ORF1b)_372-392 | VFDEISMATNYDLSVVNARLR |
| 23 | NSP13(ORF1b)_394-414 | KHYVYIGDPAQLPAPRTLLTK |
| 24 | NSP13(ORF1b)_522-542 | ASKILGLPTQTVDSSQGSEYD |
| 25 | NSP14(ORF1b)_175-195 | KNLSDRVVFVLWAHGFELTSM |
| 26 | NSP14(ORF1b)_228-248 | HHSIGFDYVYNPFMIDVQQWG |
| 27 | NSP14(ORF1b)_320-430 | ALLADKFPVLHDIGNPKAIKC |
| 28 | NSP14(ORF1b)_458-478 | QVVSDIDYVPLKSATCITRON |
| 29 | NSP14(ORF1b)_500-520 | MMISAGFSLWVYKQFDTYNLW |
| 30 | NSP14(ORF1b)_504-524 | AGFSLWVYKQFDTYNLWNTFT |
| 31 | NSP15(ORF1b)_292-312 | CSVIDLLLDDFVEIIKSQDLS |
| 32 | NSP15(ORF1b)_41-61 | ELFENKTTLPVNVAFELWAKR |
| 33 | NSP16(ORF1b)_53-73 | YLNTLTLAVPYNMRVIHFGAG |
| 34 | NSP3(ORF1a)_1-21 | APTKVTFGDDTVIEVQGYKSV |
| 35 | NSP3(ORF1a)_1136-1156 | ELKVTFFPDLNGDVVAIDYKH |
| 36 | NSP3(ORF1a)_1504-1524 | GLVAEWFLAYILFTRFFYVLG |
| 37 | NSP3(ORF1a)_1549-1569 | LIINLVQMAPISAMVRMYIFF |
| 38 | NSP3(ORF1a)_209-229 | SFSGYLKLTDNVYIKNADIVE |
| 39 | NSP4(ORF1a)_331-351 | LTPVYSFLPGVYSVIYLYLTF |
| 40 | NSP4(ORF1a)_340-360 | GVYSVIYLYLTFYLTNDVSFL |
| 41 | NSP4(ORF1a)_36-56 | HTDFSSEIIGYKAIDGGVTRD |
| 42 | NSP4(ORF1a)_417-437 | LCTFLINKEMYLKLRSDVLLP |
| 43 | NSP5(ORF1a)_164-184 | HMELPTGVHAGTDLEGNFYGP |
| 44 | NSP6(ORF1a)_237-260 | LTLGVYDYLVSTQEFRYMNSQGLL |
| 45 | NSP8(ORF1a)_150-184 | ASALWEIQQVVDADSKIVQLSEISMDNSPNLAWPL |
| 46 | NSP8(ORF1a)_53-73 | AAMQRKLEKMADQAMTQMYKQ |
| 47 | NSP9(ORF1a)_14-34 | CAAGTTQTACTDDNALAYYNT |

TABLE 2
Nucleic acid sequences of coronavirus derived peptides.

| SEQ ID | Peptide or fragment | Nucleic acid sequence |
|---|---|---|
| 48 | E_1-21 | auguacucauucguuucggaagagacagguacguuaauaguuaauagcgu |
| | | acuucuuuuucuu |
| 49 | M_160-180 | gacaucaaggaccugccuaaagaaaucacuguugcuacaucacgaacgcuu |
| | | ucuuauuacaaa |
| 50 | M_37-57 | uuugccuaugccaacaggaauagguuuuuguauauaauuaaguuaauuuu |
| | | ccucuggcuguua |
| 51 | M_8-28 | auuaccguugaagagcuuaaaaagcuccuugaacaauggaaccuaguaaua |
| | | gguuuccuauuc |
| 52 | N_105-125 | aguccaagaugguauuucuacuaccuaggaacugggccagaagcuggacuu |
| | | cccuauggugcu |
| 53 | N_296-316 | acugauuacaaacauuggccgcaaauugcacaauuugcccccagcgcuuca |
| | | gcguucuucgga |
| 54 | N_302-322 | ccgcaaauugcacaauuugcccccagegcuucageguucuucggaaugucgc |
| | | gcauuggcaug |
| 55 | NS6(ORF6)_1-21 | auguuucaucucguugacuuucagguuacuauagcagagauauuacuaauu |
| | | auuaugaggacu |
| 56 | NSP1(ORF1a)_52-72 | ggcuuaguagaaguugaaaaaggcguuuugccucaacuugaacagcccuau |
| | | guguucaucaaa |
| 57 | NSP12(ORF1b)_114- | auaucacgucaacgucuuacuaaauacacaauggcagaccucgucuaugcu |
| | 134 | uuaaggcauuuu |
| 58 | NSP12(ORF1b)_231- | guuccuguuguagauucuuauuauucauuguuaaugccuauauuaaccuug |
| | 251 | accagggcuuua |
| 59 | NSP12(ORF1b)_332- | aaaauauuuguugaugguguuccauuuguaguuucaacuggauaccacuuc |
| | 352 | agagagcuaggu |
| 60 | NSP12(ORF1b)_501- | ucagcugguuuuccauuuaauaaaugggguaaggcuagacuuuauuauga |
| | 521 | uucaaugaguuau |
| 61 | NSP12(ORF1b)_559- | ggugucucuaucuguaguacuaugaccaauagacaguuucaucaaaaauua |
| | 579 | uugaaaucaaua |
| 62 | NSP12(ORF1b)_616- | gguugggauuauccuaaaugugauagagccaugccuaacaugcuuagaauu |
| | 636 | auggccucacuu |
| 63 | NSP12(ORF1b)_64-84 | aauuuaauugauucuuacuuuguaguuaagagacacacuuucucuaacuac |
| | | caacaugaagaa |
| 64 | NSP12(ORF1b)_680- | accucaucaggagaugccacaacugcuuaugcuaauaguguuuuuaacauu |
| | 700 | ugucaagcuguc |
| 65 | NSP12(ORF1b)_852- | gguacacuuaugauugaacgguucgugucuuuagcuauagaugcuuaccca |
| | 872 | cuuacuaaacau |
| 66 | NSP12(ORF1b)_886- | caauacauaagaaagcuacaugaugaguuaacaggacacauguuagacaug |
| | 906 | uauucuguuaug |
| 67 | NSP13(ORF1b)_131- | aagcuuuuugcagcagaaacgcucaaagcuacugaggagacauuuaaacug |
| | 151 | ucuuaugguauu |
| 68 | NSP13(ORF1b)_228- | acaucacauacaguaaugccauuaagugcaccuacacuagugccacaagag |
| | 248 | cacuauguuaga |
| 69 | NSP13(ORF1b)_372- | gucuuugaugaaauuucaauggccacaaauuaugauuugaguguugucaa |
| | 392 | ugccagauuacgu |
| 70 | NSP13(ORF1b)_394- | aagcacuauguguacauuggcgacccugcucaauuaccugcaccacgcaca |
| | 414 | uugcuaacuaag |
| 71 | NSP13(ORF1b)_522- | gccucaaagauuuugggacuaccaacucaaacuguugauucaucacagggc |
| | 542 | ucagaauaugac |
| 72 | NSP14(ORF1b)_175- | aaaaaucucucugacagagucguauuugucuuaugggcacauggcuuugag |
| | 195 | uugacaucuaug |
| 73 | NSP14(ORF1b)_228- | caucauucuauuggauuugauuacgucuauaauccguuuaugauugaugu |
| | 248 | ucaacaauggggu |
| 74 | NSP14(ORF1b)_320- | gcauuauuagcagacaaauucccaguucuucacgacauugguaacccuaaa |
| | 430 | gcuauuaagugu |
| 75 | NSP14(ORF1b)_458- | caaguagugucagauauagauuauguaccacuaaagucugcuacguguaua |
| | 478 | acacguugcaau |
| 76 | NSP14(ORF1b)_500- | augaugaucucagcuggcuuuagcuuguggguuuacaaacaauuugauacu |
| | 520 | uauaaccucugg |
| 77 | NSP14(ORF1b)_504- | gcuggcuuuagcuuguggguuuacaaacaauuugauacuuauaaccucugg |
| | 524 | aacacuuuuaca |
| 78 | NSP15(ORF1b)_292- | uguucuguuauugauuuauuacuugaugauuuuguugaaauaauaaaauc |
| | 312 | ccaagauuuaucu |
| 79 | NSP15(ORF1b)_41-61 | gaauuguuugaaaaaaaacaacauuaccuguuaauguagcauuugagcu |
| | | uugggcuaagcgc |
| 80 | NSP16(ORF1b)_53-73 | uauuuaaacacauuaacauuagcuguacccuauaauaugagaguuauacau |
| | | uuuggugcuggu |
| 81 | NSP3(ORF1a)_1-21 | gcaccaacaaagguuacuuuuggugaugacacugugauagaagugcaaggu |
| | | uacaagagugug |
| 82 | NSP3(ORF1a)_1136- | gagcuuaaaguuacauuuuucccugacuuaaauggugaugugguggcuau |
| | 1156 | ugauuauaaacac |
| 83 | NSP3(ORF1a)_1504- | ggcuuaguugcagagugguuuuuggcauauauuuuuucacuagguuuuu |
| | 1524 | cuauguacuugga |
| 84 | NSP3(ORF1a)_1549- | uuaauaauuaaucuuguacaaauggccccgauuucagcuaugguuagaaug |
| | 1569 | uacaucuucuuu |
| 85 | NSP3(ORF1a)_209-229 | aguuuuagugguuauuuaaaacuuacugacaauguauacauuaaaaaugc |
| | | agacauuguggaa |
| 86 | NSP4(ORF1a)_331-351 | uuaacaccaguuuacucauucuuaccugguguuuauucuguuuuuacuug |
| | | uacuugacauuu |
| 87 | NSP4(ORF1a)_340-360 | gguguuuauucuguuauuuacuuguacuugacauuuuaucuuacuaauga |
| | | uguuucuuuuuua |
| 88 | NSP4(ORF1a)_36-56 | cauacugacuuuucaagugaaaucauaggauacaaggcuauugauggugg |
| | | ugucacucgugac |
| 89 | NSP4(ORF1a)_417-437 | cugugcaccuuuuuguuaaauaaagaaauguaucuaaaguugcguaguga |
| | | ugugcuauuaccu |
| 90 | NSP5(ORF1a)_164-184 | cauauggaauuaccaacuggaguucaugcuggcacagacuuagaagguaac |
| | | uuuuauggaccu |
| 91 | NSP6(ORF1a)_237-260 | cugacucuugguguuuaugauuacuuaguuucuacacaggaguuuagaua |
| | | uaugaauucacagggacuacuc |
| 92 | NSP8(ORF1a)_150-184 | gcaucagcauugugggaaauccaacagguuguagaugcagauaguaaaau |
| | | uguucaacuuagugaaauuaguauggacaauucaccuaauuuagcauggcc |
| | | ucuu |
| 93 | NSP8(ORF1a)_53-73 | gcagccaugcaacguaaguuggaaaagauggcugaucaagcuaugacccaa |
| | | auguauaaacag |
| 94 | NSP9(ORF1a)_14-34 | ugugcugccgguacuacacaaacugcuugcacugaugacaaugcguuagcu |
| | | uacuacaacaca |

In the context of the present invention, the RNA sequences of the above table may also be replaced by the corresponding DNA sequences in which ‘U’ is replaced by ‘T’ in the sequences as defined herein.

### EXAMPLES

The invention is illustrated by the following non-limiting examples

### Example 1: Prediction of Conserved, Immunogenic T Cell Epitopes of SARS-COV-2

SARS-COV-2 conservation and epitope prediction multiepitope construction (MyNEO) (Table 2).

In silico predictions were made to determine which SARS-COV-2 antigens are likely to be processed, transported up to the MHC molecule, and stably bind to its components as well as elicit an immune response. Predictions were made for each protein expressed by SARS-COV-2. For each protein, every possible peptide of length 9, 10 or 11 amino acids was analysed for presentation likelihood on MHC class I alleles. The MHC alleles for presentation prediction were chosen based on their prevalence in the human population. Furthermore, for each peptide predicted to be presented on MHC class I, predictions were made on the likelihood of the presented peptide to elicit a T cell response.

Additionally, to peptide presentation and immunogenicity prediction, sequence conservation analysis was performed to identify protein regions highly conserved among SARS-COV-2 variants identified in people with a SARS-COV-2 infection. There are evident advantages in prioritising epitopes located in conserved protein regions, as it ensures broad efficacy of the final vaccine against different SARS-CoV-2 strains that have been identified already. Also, due to the inherent link between conservation and functional importance, epitope selection based on evolutionary conservation minimises the risk of epitope escape as the virus is transmitted through human populations and accumulates new mutations.

The level of conservation was calculated based on genomic sequence data sampled in people with a SARS-COV-2 infection. Conservation was calculated in a time-resolved and window-wise manner. For a specific window along a SARS-COV-2 protein sequence, average sequence conservation was calculated with more recent variants weighted higher to take viral evolution into account.

For the selection of the coronavirus derived peptides for the vaccine, a conglomerative scoring approach was used to increase the chances of eliciting more potent clonotypes and more effectively prevent T-cell escape. Peptides were prioritized depending on their overall level of conservation and the number and quality of the epitopes they contain, prioritising peptide regions containing multiple targetable antigens.

In the final constructs, the selected peptides were concatenated into one long sequence. It is possible for neoepitopes to arise at the junction between the peptide sequences, so called junctional epitopes. Such junctional epitopes could interfere with the immune reaction to relevant epitopes in favour of irrelevant junctional epitopes. To reduce the potential negative effect of these epitopes the windows were ordered in way that minimizes the number of predicted junctional epitopes.

### Example 2: Preparation of RNA Constructs

### Preparation of RNA Constructs

DNA sequences encoding different concatenated nucleic acid sequences of coronavirus derived peptides as defined in Table 2 were cloned in frame to a LAMP1 derived signal peptide and the DC-LAMP sequence in order to optimize processing and presentation to MHC class I and class II. Since lengthy constructs (e.g. >11 Kb) are not well expressed, it is also possible to encoded the epitopes on two separate mRNAs constructs. For example, construct 2.2 which is 2 Kb, can also be generated in two separate constructs 2.2.1 (1 Kb) and construct 2.2.2 (1 Kb); or analogously, construct 2.4 (2 Kb) can also be generated in two separate constructs 2.4.1 and construct 2.4.2). The resulting DNA sequences as defined in Table 3 were prepared by modifying the wild type or reference encoding DNA sequences for stabilization and expression optimization. Sequences were introduced into a DNA vector comprising stabilizing 5′-UTR and 3′-UTR sequences and additionally comprising a stretch of at least 90 adenosines.

The obtained plasmid DNA construct were transformed and propagated in bacteria using common protocols known in the art. Eventually the purified and linearized plasmid DNA constructs were used for subsequent RNA in vitro transcription. In vitro transcription using T7 RNA polymerase was performed in the presence of a nucleotide mixture comprising N1-methyl-pseudouridine and cap1 analog under suitable buffer conditions. N1-methyl-pseudouridine was incorporated during the in vitro transcription to improve expression and reduce detrimental innate immune activation by the mRNA constructs themselves. The obtained RNA constructs were purified and used for in vitro and in vivo experiments.

### Expression Analysis of Concatenated T Cell Epitope RNA Constructs Using Western Blot

For the analysis of epitope construct expression, Hela cells were transfected with unformulated mRNA using Lipofectamine MessengerMAX as the transfection agent. Hela cells were seeded in a 6-well plate at a density of 320,000 cells/well. Hela cells were transfected with 2 μg unformulated mRNA using Lipofectamine MessengerMAX (Invitrogen). The mRNA constructs prepared according to Example 2 and listed in Table 3 were used in the experiment, including a negative control (water for injection). 24 h post transfection, Hela cells are detached by trypsin, harvested, and cell lysates are prepared. Cell lysates were subjected to SDS-PAGE followed by western blot detection. Western blot analysis was performed using an anti-DC-LAMP protein antibody used in combination with a suitable secondary antibody.

### Results Example 2

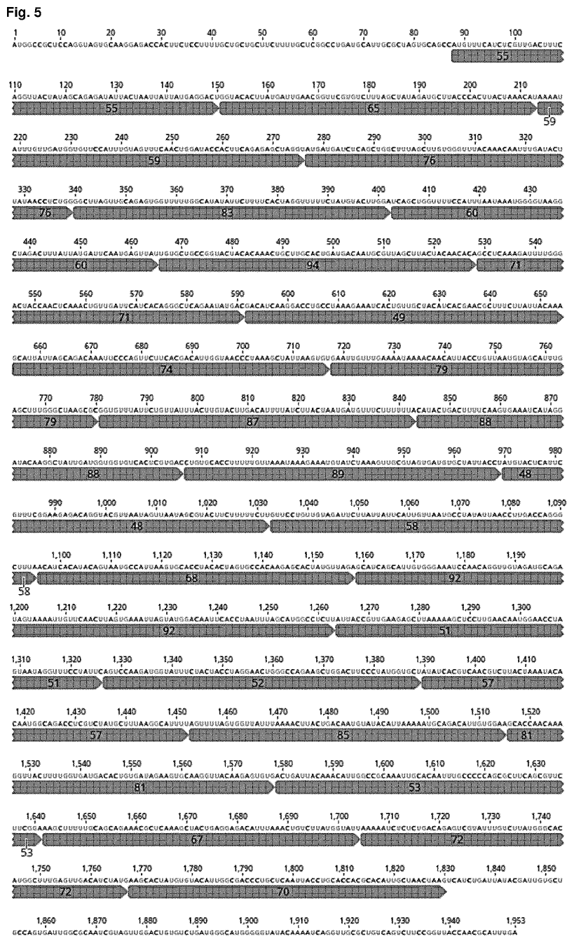

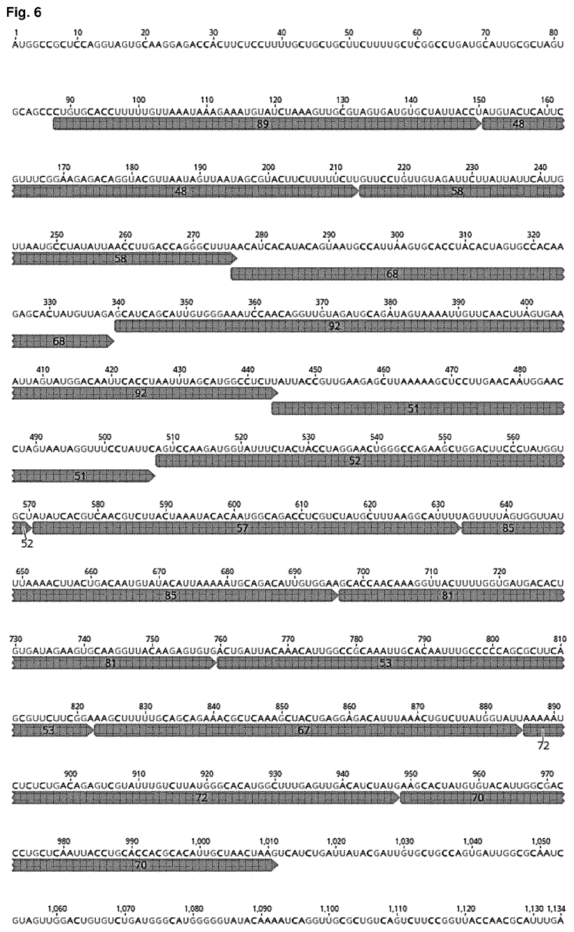

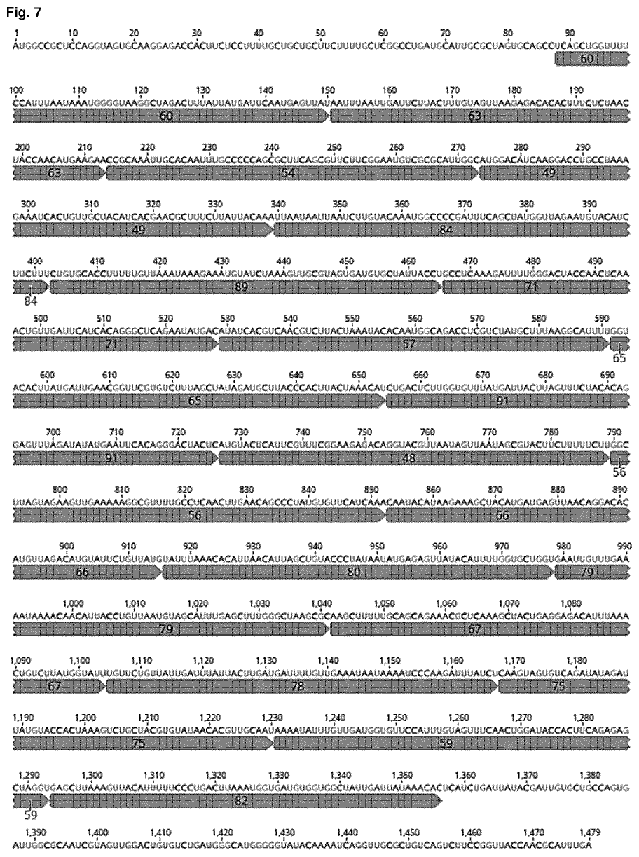

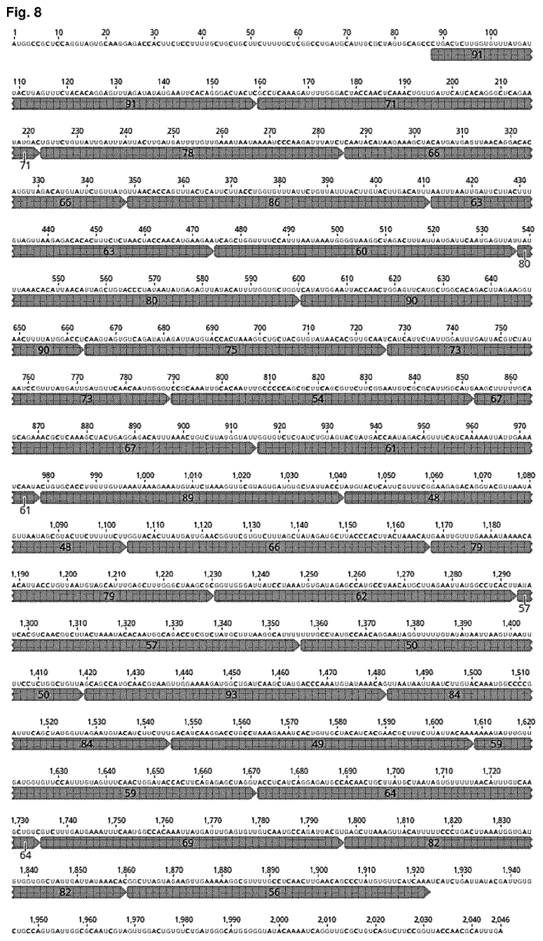

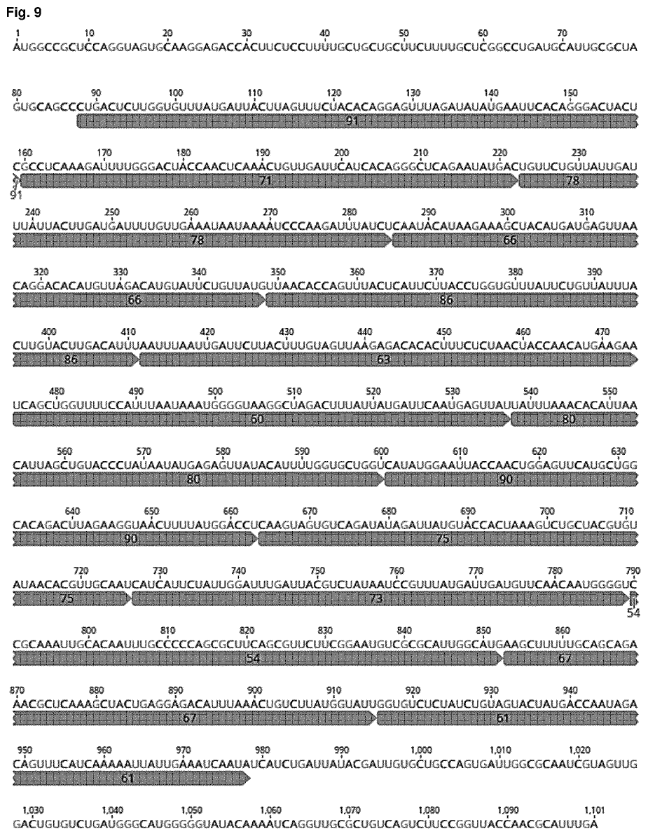

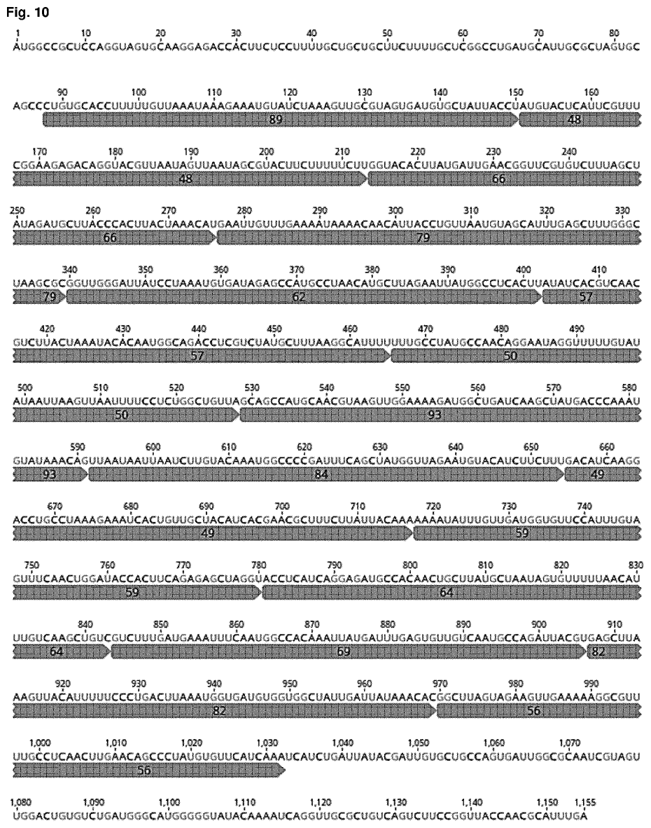

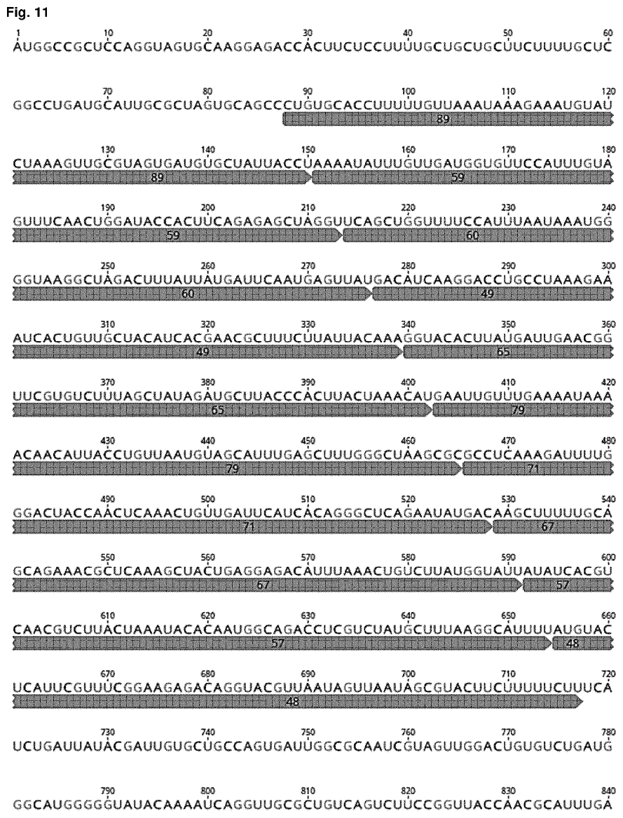

The used mRNA constructs resulted in a detectable expression that varied dependent on the sequence optimization (western blots not shown). Optimized RNA sequences of preferred constructs are listed in Table 4 while non-optimized RNA sequences of constructs are listed in Table 3 and may comprise the following SEQ IDs:
1. SARS-COV-2 construct 2.1_DCL (SEQ ID NO: 95): a multi-epitope construct comprising nucleic acid sequence: SEQ ID NO: 55, 89, 57, 53, 49, 92, 70, 71, 48, 74, 79, 77, 87, 65, 59, 88, 72, 60, 81 (see FIG. 4).
2. SARS-COV-2 construct 2.2_DCL (SEQ ID NO: 96): a multi-epitope construct comprising nucleic acid sequence: SEQ ID NOs: 55, 65, 59, 76, 83, 60, 94, 71, 49, 74, 79, 87, 88, 89, 48, 58, 68, 92, 51, 52, 57, 85, 81, 53, 67, 72, 70 (see FIG. 5).
3. SARS-COV-2 construct 2.2.1_DCL (SEQ ID NO: 97): a multi-epitope construct comprising nucleic acid sequence: SEQ ID NOs: 55, 65, 59, 76, 83, 60, 94, 71, 49, 74, 79, 87, 88 (see FIG. 6).
4. SARS-COV-2 construct 2.2.2_DCL (SEQ ID NO: 98): a multi-epitope construct comprising of nucleic acid sequence: SEQ ID NOs: 89, 48, 58, 68, 92, 51, 52, 57, 85, 81, 53, 67, 72, 70 (see FIG. 7).
5. SARS-COV-2 construct 2.3_DCL (SEQ ID NO: 99): a multi-epitope construct comprising nucleic acid sequence: SEQ ID NOs: 60, 63, 54, 49, 84, 89, 71, 57, 65, 91, 48, 56, 66, 80, 79, 67, 78, 75, 59, 82 (see FIG. 8).
6. SARS-COV-2 construct 2.4_DCL (SEQ ID NO: 100): a multi-epitope construct comprising nucleic acid sequence: SEQ ID NOs: 91, 71, 78, 66, 86, 63, 60, 80, 90, 75, 73, 54, 67, 61, 89, 48, 65, 79, 62, 57, 50, 93, 84, 49, 59, 64, 69, 82, 56 (see FIG. 9).
7. SARS-COV-2 construct 2.4.1_DCL (SEQ ID NO: 101): a multi-epitope construct comprising nucleic acid sequence: SEQ ID NOs: 91, 71, 78, 66, 86, 63, 60, 80, 90, 75, 73, 54, 67, 61 (see FIG. 10).
8. SARS-COV-2 construct 2.4.2_DCL (SEQ ID NO: 102): a multi-epitope construct comprising nucleic acid sequence: SEQ ID NOs: 89, 48, 65, 79, 62, 57, 50, 93, 84, 49, 59, 64, 69, 82, 56 (see FIG. 11).
9. SARS-COV-2 construct 2.5_DCL (SEQ ID NO: 103): a multi-epitope construct comprising nucleic acid sequence: SEQ ID NOs: 89, 59, 60, 49, 65, 79, 71, 67, 57, 48 (see FIG. 12).

### Example 3: Vaccination of Mice with mRNA Encoded SARS-COV-2 Epitopes

### Preparation of RNA Constructs

SARS-COV-2 epitope constructs are prepared as described in Example 1 and formulated with LNPs prior to use in in vivo vaccination experiments.

### Vaccination of Mice and Flow Cytometry

Female BALB/c mice (6-8 weeks old) are injected intramuscularly with mRNA vaccine compositions at dose of 5 μg. As a negative control, one group of mice is vaccinated with buffer. All animals are vaccinated on day 0 and day 21.

Splenocytes from vaccinated mice are isolated according to a standard protocol know in the art. Briefly, isolated spleens are grinded through a cell strainer and washed in PBS followed by red blood cell lysis. After an extensive washing step with PBS, splenocytes in RPMI are seeded into 96-well plates (2×10⁶ cells per well). Cells are stimulated with a mixture of specific peptide epitopes (2 μg/ml of each peptide) matching the epitopes in the mRNA constructs for 5 hours at 37° C. in the presence of a protein transport inhibitor. After stimulation, cells are washed and stained for intracellular cytokines using the Cytofix/Cytoperm reagent (BD Biosciences) according to the manufacturer's instructions. The following antibodies are used for staining: Thy1.2-Alexa700 (BioLegend), CD4-FITC (BioLegend), CD8-V450 (BD Biosciences), CD107a-BV711 (BD Biosciences) and IFNγ-PE (BD Biosciences). Zombie Aqua is used to distinguish live/dead cells (Invitrogen). Cells are acquired using a Attune flow cytometer (Thermo Fisher Scientific). Flow cytometry data is analyzed using FlowJo software package (Tree Star, Inc.)

### Results Example 3

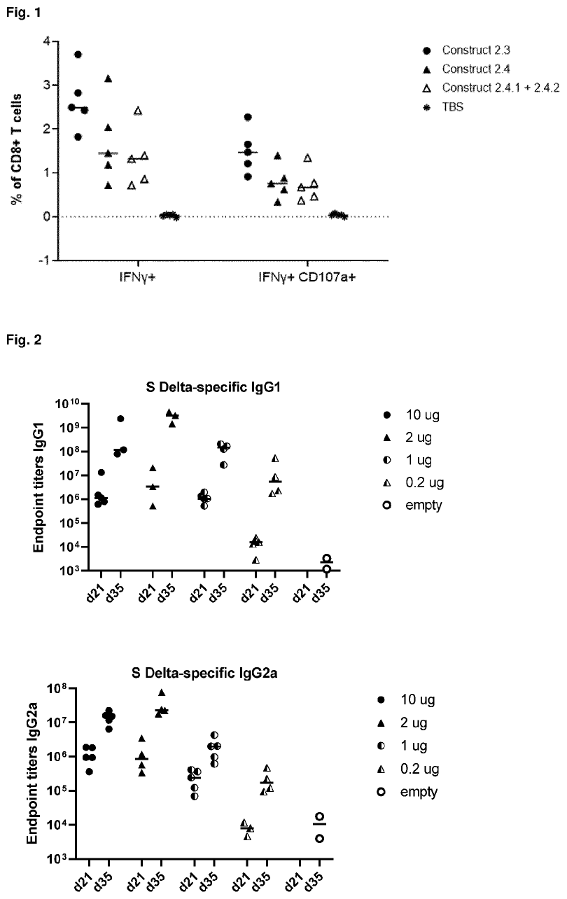

As shown in FIG. 1 the vaccination with mRNA encoding concatenated epitopes derived from conserved regions of the non-structural proteins of SARS-COV-2 induced strong epitope-specific CD8 T cell responses after a prime/boost vaccination with mRNA-LNPs.

### Example 4: Vaccination of Mice Using mRNA Construct 2.3 Opt2

In this example, a combination vaccine strategy was envisaged.

RNA constructs were prepared in accordance with the details of example 2, vaccination and analysis of the results was performed in accordance with the details of example 3.

The humoral response is elicited by a full-length SARS-COV-2 Spike mRNA and the T-cell response by the mRNA coding for a concatenated protein containing epitopes coded by the whole genome of the virus (construct 2.3 opt2-SEQ ID NO:117). Herein, the two mRNA constructs are combined in vivo. A dose response approach is chosen where equimolar ratios of the two components are tested from 0.2 μg each to 10 μg each according to the following table:

| | | | Treatment | | |
|---|---|---|---|---|---|
| RNA | Mod | Dose/Volume | days | Collection | Mice |
| Full length Spike | N1mψ- | 50 μl/10 μg each | D 0/d 21 im | Blood 6 h | 5 |
| mRNA + construct 2.3 | UTP | | | pi, d 35 | |
| Full length Spike | N1mψ- | 50 μl/2 μg each | D 0/d 21 im | Blood 6 h | 5 |
| mRNA + construct 2.3 | UTP | | | pi, d 35 | |
| Full length Spike | N1mψ- | 50 μl/1 μg each | D 0/d 21 im | Blood 6 h | 5 |
| mRNA + construct 2.3 | UTP | | | pi, d 35 | |
| Full length Spike | N1mψ- | 50 μl/0.2 μg each | D 0/d 21 im | Blood 6 h | 5 |
| mRNA + construct 2.3 | UTP | | | pi, d 35 | |
| Empty LNP | | Equivalent to 50 | D 0/d 21 im | Blood 6 h | 5 |
| | | ml/10 mg each | | pi, d 35 | |
| Buffer | | | D 0/d 21 im | Blood 6 h | 5 |
| | | | | pi, d 35 | |

### Results

Very mild transient weight loss (maximum 5%) occurred after each vaccination event (prime or boost) (data not shown). The combination of antigens in the mRNA vaccine candidate does not give rise to increased signs of toxicity even at higher doses (data not shown).

We observed a clear Induction of both IgG1 and IgG2a responses after vaccination, with a specific boost effect from the second vaccination (FIG. 2). The dose range effect is more apparent for the lower doses. Plateau effect for vaccination doses was observed for doses higher than 1 μg.

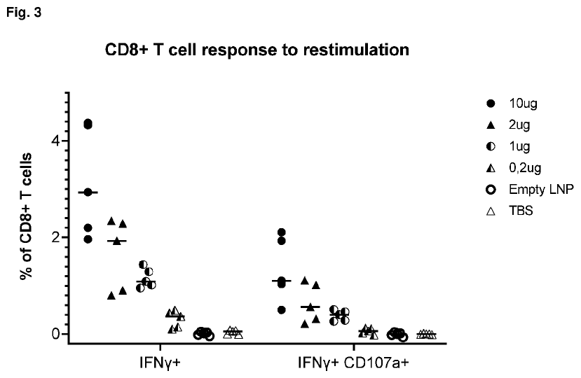

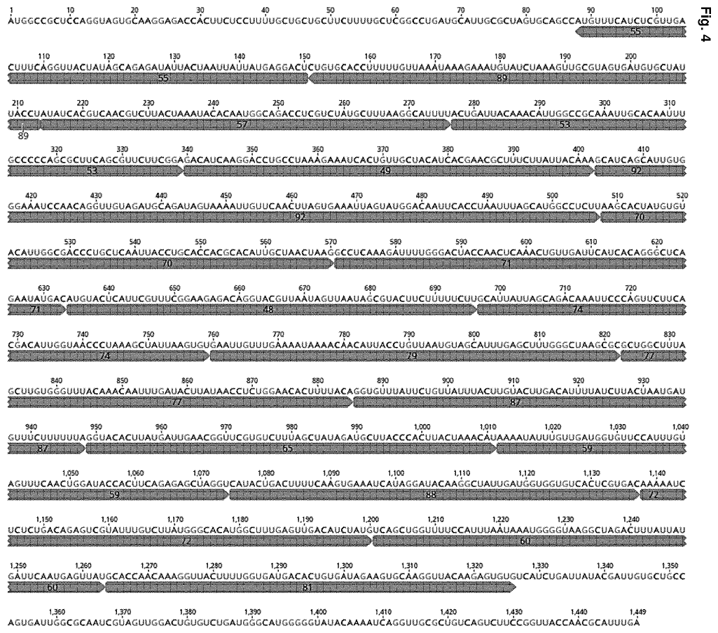

Furthermore, a dose-response increase in multifunctional CD8+ T cell activation can be seen in all the doses tested (FIG. 3).

### CONCLUSIONS

There is hardly any visible toxicity to see from the combination of mRNAs. Almost all cytokines tested, especially INF-Type II associated cytokines increased after boost, with a clear dose-response effect.

High S-specific IgG1 titers are observed for both d21 and d35 with good induction from d21 to d35. An apparent plateau effect was observed for all doses above 1 μg.

Finally, we observed a clear dose-range effect for the CD8+ T cell response.

Hence these data evidence that the constructs as disclosed therein have potential in vaccination strategies against COVID infections.

TABLE 3
Non-optimized RNA sequences of multi-epitope constructs.

| SEQ | | |
|---|---|---|
| ID | Name | Sequence |
| 95 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCAUGUUUCAUCUCGUUGACUUUCA |
| | construct | GGUUACUAUAGCAGAGAUAUUACUAAUUAUUAUGAGGACUCUGUGCACCUUUUUG |
| | 2.1_DCL | UUAAAUAAAGAAAUGUAUCUAAAGUUGCGUAGUGAUGUGCUAUUACCUAUAUCAC |
| | | GUCAACGUCUUACUAAAUACACAAUGGCAGACCUCGUCUAUGCUUUAAGGCAUUU |
| | | UACUGAUUACAAACAUUGGCCGCAAAUUGCACAAUUUGCCCCCAGCGCUUCAGCG |
| | | UUCUUCGGAGACAUCAAGGACCUGCCUAAAGAAAUCACUGUUGCUACAUCACGAA |
| | | CGCUUUCUUAUUACAAAGCAUCAGCAUUGUGGGAAAUCCAACAGGUUGUAGAUGC |
| | | AGAUAGUAAAAUUGUUCAACUUAGUGAAAUUAGUAUGGACAAUUCACCUAAUUUA |
| | | GCAUGGCCUCUUAAGCACUAUGUGUACAUUGGCGACCCUGCUCAAUUACCUGCAC |
| | | CACGCACAUUGCUAACUAAGGCCUCAAAGAUUUUGGGACUACCAACUCAAACUGU |
| | | UGAUUCAUCACAGGGCUCAGAAUAUGACAUGUACUCAUUCGUUUCGGAAGAGACA |
| | | GGUACGUUAAUAGUUAAUAGCGUACUUCUUUUUCUUGCAUUAUUAGCAGACAAAU |
| | | UCCCAGUUCUUCACGACAUUGGUAACCCUAAAGCUAUUAAGUGUGAAUUGUUUGA |
| | | AAAUAAAACAACAUUACCUGUUAAUGUAGCAUUUGAGCUUUGGGCUAAGCGCGCU |
| | | GGCUUUAGCUUGUGGGUUUACAAACAAUUUGAUACUUAUAACCUCUGGAACACUU |
| | | UUACAGGUGUUUAUUCUGUUAUUUACUUGUACUUGACAUUUUAUCUUACUAAUGA |
| | | UGUUUCUUUUUUAGGUACACUUAUGAUUGAACGGUUCGUGUCUUUAGCUAUAGAU |
| | | GCUUACCCACUUACUAAACAUAAAAUAUUUGUUGAUGGUGUUCCAUUUGUAGUUU |
| | | CAACUGGAUACCACUUCAGAGAGCUAGGUCAUACUGACUUUUCAAGUGAAAUCAU |
| | | AGGAUACAAGGCUAUUGAUGGUGGUGUCACUCGUGACAAAAAUCUCUCUGACAGA |
| | | GUCGUAUUUGUCUUAUGGGCACAUGGCUUUGAGUUGACAUCUAUGUCAGCUGGUU |
| | | UUCCAUUUAAUAAAUGGGGUAAGGCUAGACUUUAUUAUGAUUCAAUGAGUUAUGC |
| | | ACCAACAAAGGUUACUUUUGGUGAUGACACUGUGAUAGAAGUGCAAGGUUACAAG |
| | | AGUGUGUCAUCUGAUUAUACGAUUGUGCUGCCAGUGAUUGGGGCAAUCGUAGUUG |
| | | GACUGUGUCUGAUGGGCAUGGGGGUAUACAAAAUCAGGUUGCGCUGUCAGUGUUC |
| | | CGGUUACCAACGCAUUUGA |
| 96 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCAUGUUUCAUCUCGUUGACUUUCA |
| | construct | GGUUACUAUAGCAGAGAUAUUACUAAUUAUUAUGAGGACUGGUACACUUAUGAUU |
| | 2.2_DCL | GAAGGGUUCGUGUCUUUAGCUAUAGAUGCUUACCCACUUACUAAACAUAAAAUAU |
| | | UUGUUGAUGGUGUUCCAUUUGUAGUUUCAACUGGAUACCACUUCAGAGAGCUAGG |
| | | UAUGAUGAUCUCAGCUGGCUUUAGCUUGUGGGUUUACAAACAAUUUGAUACUUAU |
| | | AACCUCUGGGGCUUAGUUGCAGAGUGGUUUUUGGCAUAUAUUCUUUUCACUAGGU |
| | | UUUUCUAUGUACUUGGAUCAGCUGGUUUUCCAUUUAAUAAAUGGGGUAAGGCUAG |
| | | ACUUUAUUAUGAUUCAAUGAGUUAUUGUGCUGCCGGUACUACACAAACUGCUUGC |
| | | ACUGAUGACAAUGCGUUAGCUUACUACAACACAGCCUCAAAGAUUUUGGGACUAC |
| | | CAACUCAAACUGUUGAUUCAUCACAGGGCUCAGAAUAUGACGACAUCAAGGACCU |
| | | GCCUAAAGAAAUCACUGUUGCUACAUCACGAACGCUUUCUUAUUACAAAGCAUUA |
| | | UUAGCAGACAAAUUCCCAGUUCUUCACGACAUUGGUAACCCUAAAGCUAUUAAGU |
| | | GUGAAUUGUUUGAAAAAAAACAACAUUACCUGUUAAUGUAGCAUUUGAGCUUUGG |
| | | GCUAAGCGCGGUGUUUAUUCUGUUAUUUACUUGUACUUGACAUUUUAUCUUACUA |
| | | AUGAUGUUUCUUUUUUACAUACUGACUUUUGAAGUGAAAUCAUAGGAUACAAGGC |
| | | UAUUGAUGGUGGUGUCACUCGUGACCUGUGCACCUUUUUGUUAAAUAAAGAAAUG |
| | | UAUCUAAAGUUGCGUAGUGAUGUGCUAUUACCUAUGUACUCAUUCGUUUCGGAAG |
| | | AGACAGGUACGUUAAUAGUUAAUAGCGUACUUCUUUUUCUUGUUCCUGUUGUAGA |
| | | UUCUUAUUAUUCAUUGUUAAUGCCUAUAUUAACCUUGACCAGGGCUUUAACAUCA |
| | | CAUACAGUAAUGCCAUUAAGUGCACCUACACUAGUGCCACAAGAGCACUAUGUUA |
| | | GAGCAUCAGCAUUGUGGGAAAUCCAACAGGUUGUAGAUGCAGAUAGUAAAAUUGU |
| | | UCAACUUAGUGAAAUUAGUAUGGACAAUUCACCUAAUUUAGCAUGGCCUCUUAUU |
| | | ACCGUUGAAGAGCUUAAAAAGCUCCUUGAACAAUGGAACCUAGUAAUAGGUUUCC |
| | | UAUUCAGUCCAAGAUGGUAUUUCUACUACCUAGGAACUGGGCCAGAAGCUGGACU |
| | | UCCCUAUGGUGCUAUAUCACGUCAACGUCUUACUAAAUACACAAUGGCAGACCUC |
| | | GUCUAUGCUUUAAGGCAUUUUAGUUUUAGUGGUUAUUUAAAACUUACUGACAAUG |
| | | UAUACAUUAAAAAUGCAGACAUUGUGGAAGCACCAACAAAGGUUACUUUUGGUGA |
| | | UGACACUGUGAUAGAAGUGCAAGGUUACAAGAGUGUGACUGAUUACAAACAUUGG |
| | | CCGCAAAUUGCACAAUUUGCCCCCAGCGCUUCAGCGUUCUUCGGAAAGCUUUUUG |
| | | CAGCAGAAACGCUCAAAGCUACUGAGGAGACAUUUAAACUGUCUUAUGGUAUUAA |
| | | AAAUCUCUCUGACAGAGUCGUAUUUGUCUUAUGGGCACAUGGCUUUGAGUUGACA |
| | | UCUAUGAAGCACUAUGUGUACAUUGGCGACCCUGCUCAAUUACCUGCACCACGCA |
| | | CAUUGCUAACUAAGUCAUCUGAUUAUACGAUUGUGCUGCCAGUGAUUGGCGCAAU |
| | | CGUAGUUGGACUGUGUCUGAUGGGCAUGGGGGUAUACAAAAUCAGGUUGCGCUGU |
| | | CAGUCUUCCGGUUACCAAGGCAUUUGA |
| 97 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCAUGUUUCAUCUCGUUGACUUUCA |
| | construct | GGUUACUAUAGCAGAGAUAUUACUAAUUAUUAUGAGGACUGGUACACUUAUGAUU |
| | 2.2.1_DC | GAAGGGUUCGUGUCUUUAGCUAUAGAUGCUUACCCACUUACUAAACAUAAAAUAU |
| | L | UUGUUGAUGGUGUUCCAUUUGUAGUUUCAACUGGAUACCACUUCAGAGAGGUAGG |
| | | UAUGAUGAUCUCAGCUGGCUUUAGCUUGUGGGUUUACAAACAAUUUGAUACUUAU |
| | | AACCUCUGGGGCUUAGUUGCAGAGUGGUUUUUGGCAUAUAUUCUUUUCACUAGGU |
| | | UUUUCUAUGUACUUGGAUCAGCUGGUUUUCCAUUUAAUAAAUGGGGUAAGGCUAG |
| | | ACUUUAUUAUGAUUCAAUGAGUUAUUGUGCUGCCGGUACUACACAAACUGCUUGC |
| | | ACUGAUGACAAUGCGUUAGCUUACUACAACACAGCCUCAAAGAUUUUGGGACUAC |
| | | CAACUCAAACUGUUGAUUCAUCACAGGGCUCAGAAUAUGACGACAUCAAGGACCU |
| | | GCCUAAAGAAAUCACUGUUGCUACAUCACGAACGCUUUCUUAUUACAAAGCAUUA |
| | | UUAGCAGACAAAUUCCCAGUUCUUCACGACAUUGGUAACCCUAAAGCUAUUAAGU |
| | | GUGAAUUGUUUGAAAAAAAACAACAUUACCUGUUAAUGUAGCAUUUGAGCUUUGG |
| | | GCUAAGCGCGGUGUUUAUUCUGUUAUUUACUUGUACUUGACAUUUUAUCUUACUA |
| | | AUGAUGUUUCUUUUUUACAUACUGACUUUUCAAGUGAAAUCAUAGGAUACAAGGC |
| | | UAUUGAUGGUGGUGUCACUCGUGACUCAUCUGAUUAUACGAUUGUGCUGCCAGUG |
| | | AUUGGCGCAAUCGUAGUUGGACUGUGUCUGAUGGGCAUGGGGGUAUACAAAAUGA |
| | | GGUUGCGCUGUCAGUCUUCCGGUUACCAACGCAUUUGA |
| 98 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCCUGUGCACCUUUUUGUUAAAUAA |
| | construct | AGAAAUGUAUCUAAAGUUGCGUAGUGAUGUGCUAUUACCUAUGUACUCAUUCGUU |
| | 2.2.2_ | UCGGAAGAGACAGGUACGUUAAUAGUUAAUAGCGUACUUCUUUUUCUUGUUCCUG |
| | DCL | UUGUAGAUUCUUAUUAUUCAUUGUUAAUGCCUAUAUUAACCUUGACCAGGGCUUU |
| | | AACAUCACAUACAGUAAUGCCAUUAAGUGCACCUACACUAGUGCCACAAGAGCAC |
| | | UAUGUUAGAGCAUGAGCAUUGUGGGAAAUCCAACAGGUUGUAGAUGCAGAUAGUA |
| | | AAAUUGUUCAACUUAGUGAAAUUAGUAUGGACAAUUCACCUAAUUUAGCAUGGCC |
| | | UCUUAUUACCGUUGAAGAGCUUAAAAAGCUCCUUGAACAAUGGAACCUAGUAAUA |
| | | GGUUUCCUAUUCAGUCCAAGAUGGUAUUUCUACUACCUAGGAACUGGGCCAGAAG |
| | | CUGGACUUCCCUAUGGUGCUAUAUGACGUCAACGUCUUACUAAAUACACAAUGGC |
| | | AGACCUCGUCUAUGCUUUAAGGCAUUUUAGUUUUAGUGGUUAUUUAAAACUUACU |
| | | GACAAUGUAUACAUUAAAAAUGCAGACAUUGUGGAAGCACCAACAAAGGUUACUU |
| | | UUGGUGAUGACACUGUGAUAGAAGUGCAAGGUUACAAGAGUGUGACUGAUUACAA |
| | | ACAUUGGCCGCAAAUUGCACAAUUUGCCCCCAGCGCUUCAGCGUUCUUCGGAAAG |
| | | CUUUUUGCAGCAGAAACGCUCAAAGCUACUGAGGAGACAUUUAAACUGUCUUAUG |
| | | GUAUUAAAAAUCUCUCUGACAGAGUCGUAUUUGUCUUAUGGGCACAUGGCUUUGA |
| | | GUUGACAUCUAUGAAGCACUAUGUGUACAUUGGCGACCCUGCUCAAUUACCUGCA |
| | | CCACGCACAUUGCUAACUAAGUCAUCUGAUUAUACGAUUGUGCUGCCAGUGAUUG |
| | | GCGCAAUCGUAGUUGGACUGUGUCUGAUGGGCAUGGGGGUAUACAAAAUCAGGUU |
| | | GCGCUGUCAGUCUUCCGGUUACCAACGCAUUUGA |
| 99 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCUCAGCUGGUUUUCCAUUUAAUAA |
| | construct | AUGGGGUAAGGCUAGACUUUAUUAUGAUUGAAUGAGUUAUAAUUUAAUUGAUUCU |
| | 2.3_DCL | UACUUUGUAGUUAAGAGACACAGUUUCUCUAACUACCAACAUGAAGAACCGCAAA |
| | | UUGCACAAUUUGCCCCCAGCGCUUGAGCGUUCUUCGGAAUGUCGCGCAUUGGCAU |
| | | GGACAUCAAGGACCUGCCUAAAGAAAUCACUGUUGCUACAUCACGAACGCUUUCU |
| | | UAUUACAAAUUAAUAAUUAAUCUUGUACAAAUGGCCCCGAUUUCAGCUAUGGUUA |
| | | GAAUGUACAUCUUCUUUCUGUGCACCUUUUUGUUAAAUAAAGAAAUGUAUCUAAA |
| | | GUUGCGUAGUGAUGUGGUAUUACCUGCCUCAAAGAUUUUGGGACUACCAACUCAA |
| | | ACUGUUGAUUCAUCACAGGGCUCAGAAUAUGACAUAUCACGUCAACGUCUUAGUA |
| | | AAUACACAAUGGCAGACCUCGUCUAUGCUUUAAGGCAUUUUGGUACACUUAUGAU |
| | | UGAACGGUUCGUGUCUUUAGCUAUAGAUGCUUACCCACUUACUAAACAUCUGACU |
| | | CUUGGUGUUUAUGAUUACUUAGUUUCUACACAGGAGUUUAGAUAUAUGAAUUCAC |
| | | AGGGACUACUGAUGUACUCAUUCGUUUCGGAAGAGACAGGUACGUUAAUAGUUAA |
| | | UAGCGUACUUCUUUUUCUUGGCUUAGUAGAAGUUGAAAAAGGCGUUUUGCCUCAA |
| | | CUUGAACAGCCCUAUGUGUUCAUCAAACAAUACAUAAGAAAGCUACAUGAUGAGU |
| | | UAACAGGACACAUGUUAGACAUGUAUUCUGUUAUGUAUUUAAACACAUUAACAUU |
| | | AGCUGUACCCUAUAAUAUGAGAGUUAUACAUUUUGGUGCUGGUGAAUUGUUUGAA |
| | | AAUAAAACAACAUUACCUGUUAAUGUAGCAUUUGAGCUUUGGGCUAAGCGCAAGC |
| | | UUUUUGCAGCAGAAACGCUCAAAGCUACUGAGGAGACAUUUAAACUGUCUUAUGG |
| | | UAUUUGUUCUGUUAUUGAUUUAUUACUUGAUGAUUUUGUUGAAAUAAUAAAAUCC |
| | | CAAGAUUUAUCUCAAGUAGUGUCAGAUAUAGAUUAUGUACCACUAAAGUCUGCUA |
| | | CGUGUAUAACACGUUGCAAUAAAAUAUUUGUUGAUGGUGUUCCAUUUGUAGUUUC |
| | | AAGUGGAUACCACUUCAGAGAGCUAGGUGAGCUUAAAGUUACAUUUUUCCCUGAC |
| | | UUAAAUGGUGAUGUGGUGGCUAUUGAUUAUAAACACUCAUCUGAUUAUACGAUUG |
| | | UGCUGCCAGUGAUUGGCGCAAUCGUAGUUGGACUGUGUCUGAUGGGCAUGGGGGU |
| | | AUACAAAAUCAGGUUGCGCUGUCAGUCUUCCGGUUACCAACGCAUUUGA |
| 100 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCCUGACUCUUGGUGUUUAUGAUUA |
| | construct | CUUAGUUUCUACACAGGAGUUUAGAUAUAUGAAUUCACAGGGACUACUCGCCUCA |
| | 2.4_ | AAGAUUUUGGGACUACCAACUCAAACUGUUGAUUCAUCACAGGGCUCAGAAUAUG |
| | DCL | ACUGUUCUGUUAUUGAUUUAUUACUUGAUGAUUUUGUUGAAAUAAUAAAAUCCCA |
| | | AGAUUUAUCUCAAUACAUAAGAAAGCUACAUGAUGAGUUAACAGGACACAUGUUA |
| | | GACAUGUAUUCUGUUAUGUUAACACCAGUUUACUCAUUCUUACCUGGUGUUUAUU |
| | | CUGUUAUUUACUUGUACUUGACAUUUAAUUUAAUUGAUUCUUACUUUGUAGUUAA |
| | | GAGACACACUUUCUCUAACUACCAACAUGAAGAAUCAGCUGGUUUUCCAUUUAAU |
| | | AAAUGGGGUAAGGCUAGACUUUAUUAUGAUUCAAUGAGUUAUUAUUUAAACACAU |
| | | UAACAUUAGCUGUACCCUAUAAUAUGAGAGUUAUACAUUUUGGUGCUGGUCAUAU |
| | | GGAAUUACCAACUGGAGUUCAUGCUGGCACAGACUUAGAAGGUAACUUUUAUGGA |
| | | CCUCAAGUAGUGUCAGAUAUAGAUUAUGUACCACUAAAGUCUGCUACGUGUAUAA |
| | | CACGUUGCAAUCAUCAUUCUAUUGGAUUUGAUUACGUCUAUAAUCCGUUUAUGAU |
| | | UGAUGUUCAACAAUGGGGUCCGCAAAUUGCACAAUUUGCCCCCAGCGCUUCAGCG |
| | | UUCUUCGGAAUGUCGCGCAUUGGCAUGAAGCUUUUUGCAGCAGAAACGCUCAAAG |
| | | CUACUGAGGAGACAUUUAAACUGUCUUAUGGUAUUGGUGUCUCUAUCUGUAGUAC |
| | | UAUGACCAAUAGACAGUUUCAUCAAAAAUUAUUGAAAUCAAUACUGUGCACCUUU |
| | | UUGUUAAAUAAAGAAAUGUAUCUAAAGUUGCGUAGUGAUGUGCUAUUACCUAUGU |
| | | ACUCAUUCGUUUCGGAAGAGACAGGUACGUUAAUAGUUAAUAGCGUACUUCUUUU |
| | | UCUUGGUACACUUAUGAUUGAACGGUUCGUGUCUUUAGCUAUAGAUGCUUACCCA |
| | | CUUACUAAACAUGAAUUGUUUGAAAAUAAAACAACAUUACCUGUUAAUGUAGCAU |
| | | UUGAGCUUUGGGCUAAGCGCGGUUGGGAUUAUCCUAAAUGUGAUAGAGCCAUGCC |
| | | UAACAUGCUUAGAAUUAUGGCCUCACUUAUAUCACGUCAACGUCUUACUAAAUAC |
| | | ACAAUGGCAGACCUCGUCUAUGCUUUAAGGCAUUUUUUUGCCUAUGCCAACAGGA |
| | | AUAGGUUUUUGUAUAUAAUUAAGUUAAUUUUCCUCUGGCUGUUAGCAGCCAUGCA |
| | | ACGUAAGUUGGAAAAGAUGGCUGAUCAAGCUAUGACCCAAAUGUAUAAACAGUUA |
| | | AUAAUUAAUCUUGUACAAAUGGCCCCGAUUUCAGCUAUGGUUAGAAUGUACAUCU |
| | | UCUUUGACAUCAAGGACCUGCCUAAAGAAAUCACUGUUGCUACAUCACGAACGCU |
| | | UUCUUAUUACAAAAAAAUAUUUGUUGAUGGUGUUCCAUUUGUAGUUUCAACUGGA |
| | | UACCACUUCAGAGAGCUAGGUACCUCAUCAGGAGAUGCCACAACUGCUUAUGCUA |
| | | AUAGUGUUUUUAACAUUUGUCAAGCUGUCGUCUUUGAUGAAAUUUGAAUGGCCAC |
| | | AAAUUAUGAUUUGAGUGUUGUCAAUGCCAGAUUACGUGAGCUUAAAGUUACAUUU |
| | | UUCCCUGACUUAAAUGGUGAUGUGGUGGCUAUUGAUUAUAAACACGGCUUAGUAG |
| | | AAGUUGAAAAAGGCGUUUUGCCUCAACUUGAACAGCCCUAUGUGUUCAUCAAAUC |
| | | AUCUGAUUAUACGAUUGUGCUGCCAGUGAUUGGCGCAAUCGUAGUUGGACUGUGU |
| | | CUGAUGGGCAUGGGGGUAUACAAAAUCAGGUUGCGCUGUCAGUCUUCCGGUUACC |
| | | AACGCAUUUGA |
| 101 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCCUGACUCUUGGUGUUUAUGAUUA |
| | construct | GUUAGUUUCUACACAGGAGUUUAGAUAUAUGAAUUCACAGGGACUACUCGCCUCA |
| | 2.4.1_ | AAGAUUUUGGGACUACCAACUCAAACUGUUGAUUCAUCACAGGGCUCAGAAUAUG |
| | DCL | ACUGUUCUGUUAUUGAUUUAUUACUUGAUGAUUUUGUUGAAAUAAUAAAAUCCCA |
| | | AGAUUUAUCUCAAUACAUAAGAAAGCUACAUGAUGAGUUAACAGGACACAUGUUA |
| | | GACAUGUAUUCUGUUAUGUUAACACCAGUUUACUCAUUCUUACCUGGUGUUUAUU |
| | | CUGUUAUUUACUUGUACUUGACAUUUAAUUUAAUUGAUUCUUACUUUGUAGUUAA |
| | | GAGACACACUUUCUCUAACUACCAACAUGAAGAAUCAGCUGGUUUUCCAUUUAAU |
| | | AAAUGGGGUAAGGCUAGACUUUAUUAUGAUUCAAUGAGUUAUUAUUUAAACACAU |
| | | UAACAUUAGCUGUACCCUAUAAUAUGAGAGUUAUACAUUUUGGUGCUGGUCAUAU |
| | | GGAAUUACCAACUGGAGUUCAUGCUGGCACAGACUUAGAAGGUAACUUUUAUGGA |
| | | CCUCAAGUAGUGUCAGAUAUAGAUUAUGUACCACUAAAGUCUGCUACGUGUAUAA |
| | | CACGUUGCAAUCAUCAUUCUAUUGGAUUUGAUUACGUCUAUAAUCCGUUUAUGAU |
| | | UGAUGUUCAACAAUGGGGUCCGCAAAUUGCACAAUUUGCCCCCAGCGCUUCAGCG |
| | | UUCUUCGGAAUGUCGCGCAUUGGCAUGAAGCUUUUUGCAGCAGAAACGCUCAAAG |
| | | CUACUGAGGAGACAUUUAAACUGUCUUAUGGUAUUGGUGUCUCUAUCUGUAGUAC |
| | | UAUGACCAAUAGACAGUUUCAUCAAAAAUUAUUGAAAUCAAUAUCAUCUGAUUAU |
| | | ACGAUUGUGCUGCCAGUGAUUGGCGCAAUCGUAGUUGGACUGUGUCUGAUGGGCA |
| | | UGGGGGUAUACAAAAUCAGGUUGCGCUGUCAGUCUUCCGGUUACCAACGCAUUUG |
| | | A |
| 102 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCCUGUGCACCUUUUUGUUAAAUAA |
| | construct | AGAAAUGUAUCUAAAGUUGCGUAGUGAUGUGCUAUUACCUAUGUACUCAUUCGUU |
| | 2.4.2_DCL | UCGGAAGAGACAGGUACGUUAAUAGUUAAUAGCGUACUUCUUUUUCUUGGUACAC |
| | | UUAUGAUUGAACGGUUCGUGUCUUUAGCUAUAGAUGCUUACCCACUUACUAAACA |
| | | UGAAUUGUUUGAAAAAAAACAACAUUACCUGUUAAUGUAGCAUUUGAGCUUUGGG |
| | | CUAAGCGCGGUUGGGAUUAUCCUAAAUGUGAUAGAGCCAUGCCUAACAUGCUUAG |
| | | AAUUAUGGCCUCACUUAUAUCACGUCAACGUCUUACUAAAUACACAAUGGCAGAC |
| | | CUCGUCUAUGCUUUAAGGCAUUUUUUUGCCUAUGCCAACAGGAAUAGGUUUUUGU |
| | | AUAUAAUUAAGUUAAUUUUCCUCUGGCUGUUAGCAGCCAUGCAACGUAAGUUGGA |
| | | AAAGAUGGGUGAUCAAGCUAUGACCCAAAUGUAUAAACAGUUAAUAAUUAAUCUU |
| | | GUACAAAUGGCCCCGAUUUCAGCUAUGGUUAGAAUGUACAUCUUCUUUGACAUCA |
| | | AGGACCUGCCUAAAGAAAUCACUGUUGCUACAUCACGAACGCUUUCUUAUUACAA |
| | | AAAAAUAUUUGUUGAUGGUGUUCCAUUUGUAGUUUCAACUGGAUACCACUUCAGA |
| | | GAGCUAGGUACCUCAUCAGGAGAUGCCACAACUGCUUAUGCUAAUAGUGUUUUUA |
| | | ACAUUUGUCAAGCUGUCGUCUUUGAUGAAAUUUCAAUGGCCACAAAUUAUGAUUU |
| | | GAGUGUUGUCAAUGCCAGAUUACGUGAGCUUAAAGUUACAUUUUUCCCUGACUUA |
| | | AAUGGUGAUGUGGUGGCUAUUGAUUAUAAACACGGCUUAGUAGAAGUUGAAAAAG |
| | | GCGUUUUGCCUCAACUUGAACAGCCCUAUGUGUUCAUCAAAUCAUCUGAUUAUAC |
| | | GAUUGUGCUGCCAGUGAUUGGCGCAAUCGUAGUUGGACUGUGUCUGAUGGGCAUG |
| | | GGGGUAUACAAAAUCAGGUUGCGCUGUCAGUCUUCCGGUUACCAACGCAUUUGA |
| 103 | SARS- | AUGGCCGCUCCAGGUAGUGCAAGGAGACCACUUCUCCUUUUGCUGCUGCUUCUUU |
| | CoV-2 | UGCUCGGCCUGAUGCAUUGCGCUAGUGCAGCCCUGUGCACCUUUUUGUUAAAUAA |
| | construct | AGAAAUGUAUCUAAAGUUGCGUAGUGAUGUGCUAUUACCUAAAAUAUUUGUUGAU |
| | 2.5_DCL | GGUGUUCCAUUUGUAGUUUCAACUGGAUACCACUUCAGAGAGCUAGGUUCAGCUG |
| | | GUUUUCCAUUUAAUAAAUGGGGUAAGGCUAGACUUUAUUAUGAUUCAAUGAGUUA |
| | | UGACAUCAAGGACCUGCCUAAAGAAAUCACUGUUGCUACAUCACGAACGCUUUCU |
| | | UAUUACAAAGGUACACUUAUGAUUGAACGGUUCGUGUCUUUAGCUAUAGAUGCUU |
| | | ACCCACUUACUAAACAUGAAUUGUUUGAAAAUAAAACAACAUUACCUGUUAAUGU |
| | | AGCAUUUGAGCUUUGGGCUAAGCGCGCCUCAAAGAUUUUGGGACUACCAACUCAA |
| | | ACUGUUGAUUCAUCACAGGGCUCAGAAUAUGACAAGCUUUUUGCAGCAGAAACGC |
| | | UCAAAGCUACUGAGGAGACAUUUAAACUGUCUUAUGGUAUUAUAUCACGUCAACG |
| | | UCUUACUAAAUACACAAUGGCAGACCUCGUCUAUGCUUUAAGGCAUUUUAUGUAC |
| | | UCAUUCGUUUCGGAAGAGACAGGUACGUUAAUAGUUAAUAGCGUACUUCUUUUUC |
| | | UUUCAUCUGAUUAUACGAUUGUGCUGCCAGUGAUUGGCGCAAUCGUAGUUGGACU |
| | | GUGUCUGAUGGGCAUGGGGGUAUACAAAAUCAGGUUGCGCUGUCAGUCUUCCGGU |
| | | UACCAACGCAUUUGA |

In the context of the present invention, the RNA sequences of the above table may also be replaced by the corresponding DNA sequences in which ‘U’ is replaced by ‘T’ in the sequences as defined herein.

TABLE 4
Codon optimized RNA sequences of multi-epitope constructs.
(_opt1; _opt2 ;_opt3 refer to three differently codon-optimized
sequences of the same construct).

| SEQ | | |
|---|---|---|
| ID | Name | Sequence |
| 104 | SARS- | AUGGCUGCUCCUGGAUCUGCUAGAAGGCCUCUGCUGCUGCUUCUG |
| | CoV- | UUGCUCCUGCUGCUGGGCCUGAUGCAUUGUGCCUCUGCCGCCAUG |
| | 2_concat_ | UUCCACCUGGUGGACUUCCAAGUGACAAUCGCCGAGAUCCUGCUG |
| | 2.1_ | AUCAUCAUGCGGACCCUGUGCACCUUCCUGCUGAACAAAGAGAUG |
| | DC | UACCUGAAGCUGCGGAGCGACGUGCUGCUGCCUAUCAGCAGACAG |
| | L_opt1 | CGGCUGACCAAGUACACCAUGGCCGAUCUGGUGUACGCCCUGAGA |
| | | CACUUCACCGACUACAAGCACUGGCCCCAGAUCGCCCAGUUUGCC |
| | | CCUUCUGCCAGCGCCUUCUUCGGCGACAUUAAGGACCUCCGUAAA |
| | | GAAAUCACCGUGGCCACCAGCAGAACCCUGAGCUACUACAAGGGU |
| | | AGCGCCCUGUGGGAGAUCCAGCAGGUUGUGGAUGCCGACAGCAAG |
| | | AUGGUGCAGCUGAGCGAGAUCAGCAUGGACAACAGCCCCAAUCUG |
| | | GCCUGGCCUCUGAAGCACUACGUGUACAUCGGCGACCCUGCACAG |
| | | CUGCCCGCUCCUAGAACACUGCUGAGAAAGGCCUCCAAGAUCCUG |
| | | GGCCUGCGUACACAGACCGUGGAUAGCUCUCAGGGCAGCGAGUAC |
| | | GACAUGUACAGCUUCGUGUCCGAGGAAACCGGCACACUGAUGGUG |
| | | AACUCUGUCCUGCUGUUUCUGGCCCUGCUGGCCGACAAGUUUCCU |
| | | GUGCUGCACGACAUCGGCAACCCCAAGGCCAUUAAGUGCGAGCUG |
| | | UUCGAGAACAAGACCACACUGCCCGUGAACGUGGCCUUUGAGGUG |
| | | UGGGCCAAACGCGCCGGAUUUUCCCUGUGGGUGUACAAGCAGUUC |
| | | GACACCUACAACCUGUGGAACACCUUCACCGGCGUGUACAGCGUG |
| | | AUCUACCUGUACCUGACCUUCUACCUGACCAACGACGUGUCCUUC |
| | | CUGGGCACCCUGAUGAUCGAGAGAUUCGUGUCCCUGGCUAUCGAC |
| | | GCCUAUCCUCUGACCAAGCACAAGAUCUUCGUGGACGGCGUGCCC |
| | | UUCGUGGUGUCUACCGGCUACCACUUUAGAGAGCUGGGCCACACC |
| | | GACUUCAGCUCCGAGAUCAUCGGCUACAAGGCCAUCGACGGGGGC |
| | | GUGACCAGAGACAAGAACCUGAGCGACAGAGUGGUGUUCGUGCUG |
| | | UGGGCUCACGGAUUCGAGCUGACCUCUAUGAGCGCCGGCUUUCCC |
| | | UUCAACAAAUGGGGCAAAGCCCGGCUGUACUACGACAGCAUGAGC |
| | | UACGCCCCUACCAAAGUGACCUUUGGCGACGACACCGUGAUCGAG |
| | | GUGCAGGGCUACAAGAGCGUGUCCAGCGACUACACCAUCGUGCUG |
| | | CCUGUGAUCGGCGCCAUCGUUGUGGGACUGUGUCUGAUGGGCAUG |
| | | GGAGUGUACAAGAUCCGGCUGAGAUCCAGUCCAGGGCUACCAGAG |
| | | AAUCUGA |
| 105 | SARS- | AUGGCCGCCCCCGGCAGCGCCCGCCGCCCCCUGCUGCUGCUGCUG |
| | CoV- | CUGCUGCUGCUGCUGGGCCUGAUGCACUGCGCCAGCGCCGCCAUG |
| | 2_concat_ | UUCCACCUGGUGGACUUCCAGGUGACCAUCGCCGAGAUCCUGCUG |
| | 2.1_DC | AUCAUCAUGCGCACCCUGUGCACCUUCCUGCUGAACAAGGAGAUG |
| | L_opt2 | UACCUGAAGGUGGGGAGCGACGUGCUGCUGCCCAUCAGCCGCCAG |
| | | CGCCUGACCAAGUACACCAUGGCCGACCUGGUGUACGCCCUGCGC |
| | | CACUUCACCGACUACAAGCACUGGCCCCAGAUCGCCCAGUUCGCC |
| | | CCCAGCGCCAGCGCCUUCUUCGGGGACAUCAAGGACCUCCCCAAG |
| | | GAGAUCACCGUGGCCACCAGCCGCACCGUGAGCUACUACAAGGCC |
| | | AGCGCCCUGUGGGAGAUCCAGCAGGUGGUGGACGCCGACAGCAAG |
| | | AUCGUGCAGCUGAGCGAGAUCAGCAUGGACAACAGCCCCAACCUG |
| | | GCCUGGCCCCUGAAGCACUACGUGUACAUCGGCGACCCCGCCCAG |
| | | CUGCCCGCCCCCCGCACCCUGGUGACCAAGGCCAGCAAGAUCCUG |
| | | GGCCUGCCCACCCAGACCGUGGACAGCAGCCAGGGCAGCGAGUAC |
| | | GACAUGUACAGCUUCGUGAGCGAGGAGACCGGCACCCUGAUCGUG |
| | | AACAGCGUGGUGCUGUUCCUGGCCCUGCUGGCCGAGAAGUUCCCC |
| | | GUGCUGCACGACAUCGGCAACCCCAAGGCCAUCAAGUGCGAGCUG |
| | | UUCGAGAAGAAGACCACCCUGCCCGUGAACGUGGCCUUCGAGCUG |
| | | UGGGCCAAGCGCGCCGGCUUCAGCCUGUGGGUGUACAAGCAGUUC |
| | | GACACCUACAACCUGUGGAACACCUUCACCGGCGUGUACAGCGUG |
| | | AUCUACCUGUACCUGACCUUCUACCUGACCAACGACGUGAGCUUC |
| | | CUGGGCACCCUGAUGAUCGAGCGCUUCGUGAGCCUGGCCAUCGAC |
| | | GCCUACCCCCUGACCAAGCACAAGAUCUUCGUGGACGGCGUGCCC |
| | | UUCGUGGUGAGCACCGGCUACCACUUCCGCGAGCUGGGGGACACC |
| | | GACUUCAGCAGCGAGAUCAUCGGCUACAAGGCCAUCGACGGCGGC |
| | | GUGACCCGCGAGAAGAACCUGAGCGACCGCGUGGUGUUCGUGCUG |
| | | UGGGCCCACGGCUUCGAGCUGACCAGCAUGAGCGCCGGCUUCCCC |
| | | UUCAACAAGUGGGGCAAGGCCCGCCUGUACUACGACAGCAUGAGC |
| | | UACGCCCCCACCAAGGUGACCUUCGGCGACGACACCGUGAUCGAG |
| | | GUGCAGGGCUACAAGAGCGUGAGCAGCGACUACACCAUCGUGCUG |
| | | CCCGUGAUCGGCGCCAUCGUGGUGGGCCUGUGCCUGAUGGGCAUG |
| | | GGCGUGUACAAGAUCCGCCUGCGCUGCCAGAGCAGCGGCUACCAG |
| | | CGCAUCUAA |
| 106 | SARS- | AUGGCUGCCCCAGGCUCUGCAAGGCGGCCUCUGCUGCUGCUGUUG |
| | CoV- | CUGCUGCUGCUGCUGGGACUGAUGCACUGCGCAUCUGCCGCCAUG |
| | 2_concat_ | UUCCACCUGGUGGACUUCCAGGUGACAAUCGCUGAGAUCUUGCUG |
| | 2.1_DC | AUCAUCAUGAGAACCCUGUGCACCUUCCUGCUGAAGAAGGAGAUG |
| | L_opt3 | UACCUGAAGGUGAGGAGCGACGUGCUGCUCCCUAUUUCGAGACAG |
| | | AGACUGACAAAAUAUAGAAUGGCCGAUCUGGUGUACGCCCUGCGG |
| | | CAUUUUACCGAUUACAAGCACUGGCCCCAGAUCGCCCAGUUCGCC |
| | | CCUAGCGCCAGCGCCUUUUUCGGGGACAUUAAGGACCUCCCUAAA |
| | | GAGAUCACUGUGGCCAGCUCAAGGACCCUGUCUUACUACAAGGCC |
| | | AGCGCACUGUGGGAAAUUCAGCAGGUGGUGGACGCCGACUCUAAG |
| | | AUCGUGCAGCUGUCCGAGAUUAGCAUGGAUAAUUCACCCAAUCUG |
| | | GCCUGGCCCCUGAAACACUACGUGUACAUCGGCGAUCCCGCCCAG |
| | | CUGCCCGCCCCCCGCACCCUUCUGACAAAAGCUUCUAAGAUCCUG |
| | | GGCCUGCCUACACAGACUGUGGACAGCUCACAGGGGAGCGAGUAC |
| | | GAUAUGUACUCUUUCGUGAGCGAGGAGACAGGCACCCUGAUUGUG |
| | | AACAGCGUGCUGCUGUUCCUGGCCCUGCUGGCCGAUAAGUUUCCC |
| | | GUGCUGCACGACAUUGGCAACCCCAAGGCCAUCAAAUGCGAACUG |
| | | UUCGAGAAGAAGACCACUCUGCCCGUGAAUGUGGCCUUUGAGCUG |
| | | UGGGCUAAAAGAGCCGGCUUCUCCCUGUGGGUGUACAAGCAGUUU |
| | | GACACCUACAACCUGUGGAACACCUUUACAGGAGUCUACUCCGUG |
| | | AUCUACCUGUACCUGACCUUCUAUCUGAGAAACGACGUGAGCUUU |
| | | CUGGGAACACUGAUGAUCGAGAGAUUCGUGAGCCUGGCGAUCGAC |
| | | GCCUACCCCCUGACCAAGCACAAAAUUUUCGUCGACGGGGUGCCC |
| | | UUCGUGGUGUCCACAGGCUACCACUUCAGAGAACUGGGCCACACA |
| | | GAUUUUUCCAGUGAGAUUAUCGGGUACAAGGCUAUUGACGGCGGA |
| | | GUGACAAGAGAUAAGAAUCUGUCUGAUAGGGUGGUGUUCGUGCUG |
| | | UGGGCCCACGGAUUUGAGCUGACCUCUAUGUCAGCCGGAUUCCCU |
| | | UUUAACAAAUGGGGCAAGGCUCGGCUGUACUACGACUCAAUGUCC |
| | | UACGCCCCCACCAAGGUGACAUUCGGCGAUGACACAGUGAUCGAG |
| | | GUGGAGGGAUACAAGUCCGUGUCUAGCGACUAUACAAUUGUGCUC |
| | | CCUGUGAUCGGCGCCAUCGUGGUGGGGCUGUGCCUCAUGGGCAUG |
| | | GGCGUGUAUAAAAUCCGGCUGAGAUGUCAGAGCAGCGGCUAUCAG |
| | | CGUAUCUAG |
| 107 | SARS- | AUGGCUGCUCCUGGAUCUGCUAGAAGGCCUCUGCUGCUGCUUCUG |
| | CoV- | UUGCUCCUGCUGCUGGGCCUGAUGCAUUGUGCCUCUGCCGCCAUG |
| | 2_concat_ | UUCCACCUGGUGGACUUCCAAGUGACAAUCGCCGAGAUCCUGCUG |
| | 2.2_ | AUCAUCAUGCGGACCGGCACACUGAUGAUCGAGAGAUUCGUGUCC |
| | DC | CUGGCUAUCGACGCCUAUCCUCUGACCAAGCACAAGAUCUUCGUG |
| | L_opt1 | GACGGCGUGCCCUUCGUGGUGUCUACCGGCUACCACUUCAGAGAA |
| | | CUGGGAAUGAUGAUCUCCGCCGGCUUCAGCCUGUGGGUGUACAAG |
| | | CAGUUCGACACCUACAACCUGUGGGGCCUCGUGGCCGAAUGGUUC |
| | | CUGGCCUACAUCCUGUUCACCCGGUUCUUCUACGUGCUGGGCUCU |
| | | GCCGGCUUUCCCUUCAACAAAUGGGGCAAAGCCCGGCUGUACUAC |
| | | GACAGCAUGAGCUACUGUGCCGCCGGAACCACACAGACGGCCUGC |
| | | ACAGAUGAUAACGCCCUGGCCUAUUACAACACCGCCAGCAAGAUC |
| | | CUGGGCCUGCCUACACAGACAGUGGAUAGCUCUCAGGGCAGCGAG |
| | | UACGACGACAUCAAGGACCUCCCUAAAGAAAUCACCGUGGCCACC |
| | | AGCAGAACCCUGAGCUACUAUAAGGCCCUGCUGGCCGACAAGUUC |
| | | CCCGUGCUGCACGAUAUCGGCAACCCCAAGGCCAUCAAGUGCGAG |
| | | CUGUUCGAGAACAAGACCACACUGCCCGUGAACGUGGCCUUUGAG |
| | | CUGUGGGCUAAACGGGGCGUGUACAGCGUGAUCUACCUGUACCUG |
| | | ACCUUCUACCUGACCAACGACGUGUCCUUCCUGCACACCGACUUC |
| | | AGGAGCGAGAUCAUCGGCUACAAGGCCAUUGAUGGGGGCGUGACC |
| | | AGAGAUCUGUGCACCUUCCUGCUGAACAAAGAGAUGUACCUGAAG |
| | | CUGCGGAGCGACGUGCUGCUGCCCAUGUACAGCUUUGUGUCCGAG |
| | | GAAACAGGCACCCUGAUCGUGAACUCUGUCCUGCUGUUCCUGGUG |
| | | CCUGUGGUGGACAGCUACUACAGCGUGCUGAUGCCCAUCCUGACA |
| | | CUGACAAGAGCCCUGACCAGCCACACCGUGAUGCCUCUGUCUGCU |
| | | CCUACACUGGUGCCCCAAGAGCACUAUGUGCGGGCAUCUGCCCUG |
| | | UGGGAGAUCCAGCAAGUGGUGGACGCCGACUCCAAGAUUGUGCAG |
| | | CUGAGCGAGAUCUCCAUGGAGAACAGCCCCAAUCUGGCCUGGCCU |
| | | CUGAUCACAGUGGAAGAACUGAAGAAACUGCUGGAAGAGUGGAAC |
| | | CUCGUGAUCGGCUUCCUGUUCAGCCCCAGAUGGUACUUCUACUAC |
| | | CUCGGCACAGGACCCGAAGCCGGACUUCCUUAUGGCGCUAUCUCC |
| | | CGGCAGCGGCUGACCAAGUAUACCAUGGCCGAUCUGGUGUACGCC |
| | | CUGCGGCACUUUAGCUUCAGCGGCUAUCUGAAGCUGACCGAGAAC |
| | | GUGUACAUCAAGAACGCCGACAUCGUGGAAGCCCCUACCAAAGUG |
| | | ACCUUCGGCGACGAUACCGUGAUCGAGGUGCAGGGCUACAAGAGC |
| | | GUGACCGACUAGAAGCACUGGCCCCAGAUCGCCCAGUUUGCCCCU |
| | | UCUGCCAGCGCCUUCUUCGGCAAGCUGUUUGCCGCCGAGACACUG |
| | | AAGGCCACCGAAGAGACAUUCAAGCUGAGCUACGGCAUCAAGAAU |
| | | CUGAGCGACAGAGUGGUGUUCGUGCUGUGGGCCCAUGGAUUCGAG |
| | | CUGACCAGCAUGAAGCACUACGUCUACAUCGGCGACCCCGCAGAA |
| | | CUGCCCGCUCCUAGAACAGUGCUGACCAAAAGCAGCGAGUACACC |
| | | AUCGUGCUGCCUGUGAUCGGCGCCAUCGUUGUGGGACUGUGUCUG |
| | | AUGGGCAUGGGAGUGUACAAGAUCCGGCUGAGAUGCCAGUCCAGC |
| | | GGCUACCAGAGAAUCUGA |
| 108 | SARS- | AUGGCCGCCCCCGGCAGCGCCCGCCGCCCCCUGCUGCUGCUGCUG |
| | CoV- | CUGCUGCUGCUGCUGGGCCUGAUGCACUGGGCCAGCGCCGCCAUG |
| | 2_concat_ | UUCCACCUGGUGGACUUCCAGGUGACCAUCGCCGAGAUCCUGCUG |
| | 2.2_ | AUCAUCAUGCGCACCGGCACCCUGAUGAUCGAGCGCUUCGUGAGC |
| | DC | CUGGCCAUCGACGCCUACCCCCUGACCAAGCACAAGAUCUUCGUG |
| | L_opt2 | GACGGCGUGCCCUUCGUGGUGAGCACCGGCUACCACUUCCGCGAG |
| | | CUGGGCAUGAUGAUCAGCGCCGGCUUCAGCCUGUGGGUGUACAAG |
| | | CAGUUCGACACCUACAACCUGUGGGGCCUGGUGGCCGAGUGGUUC |
| | | CUGGCCUACAUCCUGUUCACCCGCUUCUUCUACGUGCUGGGCAGC |
| | | GCCGGCUUCCCCUUCAACAAGUGGGGCAAGGCCCGCCUGUACUAC |
| | | GACAGCAUGAGCUACUGCGCCGCCGGCACCAGCCAGACCGCCUGC |
| | | ACCGACGACAACGCCCUGGCCUACUACAACACCGCCAGCAAGAUC |
| | | CUGGGCCUGCCCACCCAGACCGUGGACAGCAGCCAGGGCAGCGAG |
| | | UACGACGACAUCAAGGACCUCCCCAAGGAGAUCACCGUGGCCACC |
| | | AGCCGCACCCUGAGCUACUACAAGGCCCUGCUGGCCGACAAGUUC |
| | | CCCGUGCUGCACGACAUCGGCAACCCCAAGGCCAUCAAGUGCGAG |
| | | CUGUUCGAGAACAAGACCACCCUGCCCGUGAACGUGGCCUUCGAG |
| | | CUGUGGGCCAAGCGCGGCGUGUACAGCGUGAUCUACCUGUACCUG |
| | | ACCUUCUACCUGACCAACGACGUGAGCUUCCUGGACACCGACUUC |
| | | AGCAGCGAGAUCAUCGGCUACAAGGCCAUCGACGGCGGCGUGACC |
| | | CGCGACCUGUGCACCUUCCUGCUGAACAAGGAGAUGUACCUGAAG |
| | | CUGGGCAGCGACGUGCUGCUGCCCAUGUACAGCUUCGUGAGCGAG |
| | | GAGACCGGCACCCUGAUCGUGAACAGCGUGCUGCUGUUCCUGGUG |
| | | CCCGUGGUGGACAGCUACUACAGCCUGCUGAUGCCCAUCCUGACC |
| | | CUGACCCGCGCCCUGACCAGCCACACCGUGAUGCCCCUGAGCGCC |
| | | CCCACCCUGGUGCCCCAGGAGCACUACGUGCGCGCCAGCGCCCUG |
| | | UGGGAGAUCCAGCAAGUGGUGGACGCCGACAGCAAGAUGGUGCAG |
| | | CUGAGCGAGAUCAGCAUGGACAACAGCCCCAACCUGGCCUGGCCC |
| | | CUGAUCACCGUGGAGGAGCUGAAGAAGCUGCUGGAGCAGUGGAAC |
| | | CUGGUGAUCGGCUUCCUGUUCAGCCCCCGCUGGUACUUCUACUAC |
| | | CUGGGCACCGGCCCCGAGGCCGGCCUGCCCUACGGGGCCAUCAGC |
| | | CGCCAGCGCCUGACCAAGUACACCAUGGCCGACCUGGUGUACGCC |
| | | CUGCGCCACUUCAGCUUCAGCGGCUACCUGAAGCUGACCGACAAC |
| | | GUGUACAUCAAGAACGCCGACAUCGUGGAGGCCCCCACCAAGGUG |
| | | ACCUUCGGCGACGACACCGUGAUCGAGGUGCAGGGCUACAAGAGC |
| | | GUGACCGACUACAAGCACUGGCCCCAGAUCGCCCAGUUCGCCCCC |
| | | AGCGCCAGCGCCUUCUUCGGCAAGCUGUUCGCCGCCGAGACCCUG |
| | | AAGGCCACCGAGGAGACCUUGAAGCUGAGCUACGGCAUCAAGAAC |
| | | CUGAGCGACCGCGUGGUGUUCGUGCUGUGGGCCCACGGCUUGGAG |
| | | CUGACCAGCAUGAAGCAGUACGUGUACAUCGGCGACCCCGCCCAG |
| | | CUGCCCGCCCCCCGCACCCUGCUGACCAAGAGCAGCGACUACACC |
| | | AUCGUGCUGCCCGUGAUCGGCGCCAUCGUGGUGGGCCUGUGCCUG |
| | | AUGGGCAUGGGGGUGUACAAGAUCCGCCUGCGCUGGCAGAGCAGC |
| | | GGCUACCAGCGCAUCUAA |
| 109 | SARS- | AUGGCCGCCCCAGGGAGCGCCCGCAGGCCUCUGCUGCUGCUGCUG |
| | CoV- | CUGCUGCUCCUGCUGGGCCUGAUGCAUUGUGCCAGCGCCGCCAUG |
| | 2_concat_ | UUCCACCUGGUGGACUUUCAGGUGACCAUUGCCGAGAUCCUGCUG |
| | 2.2_ | AUCAUCAUGGGGACCGGAACUCUGAUGAUCGAGAGGUUUGUGUCU |
| | DC | CUGGGAAUCGAUGCUUACCCAUUGACCAAACACAAGAUUUUCGUG |
| | L_opt3 | GAUGGCGUGCCCUUCGUGGUCUCAACCGGCUAUCAGUUUAGAGAG |
| | | CUGGGAAUGAUGAUCUCCGCCGGCUUCAGCCUGUGGGUCUACAAG |
| | | CAGUUCGACACUUACAAUCUGUGGGGGCUGGUCGCCGAGUGGUUC |
| | | CUGGCUUACAUCCUGUUCACACGCUUCUUCUACGUGCUGGGCUCU |
| | | GCCGGCUUUCCCUUCAAUAAGUGGGGCAAGGCUAGACUGUACUAU |
| | | GAUUCCAUGAGCUACUGUGGAGCCGGCACUACACAGACCGCCUGC |
| | | ACAGACGAUAAUGCACUGGCCUACUAUAACACAGCCUCCAAGAUC |
| | | CUGGGCCUGCCUACUCAGACCGUGGACAGUAGUCAGGGCUCUGAG |
| | | UACGACGAUAUCAAGGAUCUGCCCAAAGAGAUCACGGUGGCCACA |
| | | UCCAGAACACUCAGCUACUACAAGGCCCUGCUGGCAGAUAAAUUC |
| | | CCAGUGCUGGACGACAUCGGUAACCCUAAGGCCAUUAAAUGCGAG |
| | | CUGUUCGAAAACAAGACAACCCUGCCCGUGAACGUGGCUUUCGAG |
| | | CUGUGGGCCAAGAGGGGGGUGUAUUCUGUGAUCUACCUGUACCUG |
| | | ACAUUCUACCUGACAAAUGACGUCAGCUUCCUGCACACUGAUUUU |
| | | UCCAGCGAGAUUAUCGGCUAUAAGGCCAUUGAUGGCGGCGUGACA |
| | | CGCGAUCUGUGCACCUUCCUGCUGAAUAAGGAAAUGUACCUGAAG |
| | | CUGCGCAGCGACGUGCUGCUGCCAAUGUACAGUUUUGUGAGCGAG |
| | | GAGACGGGCACCCUGAUCGUGAACAGCGUGCUGCUGUUCCUGGUG |
| | | CCAGUGGUGGACUCAUAUUACAGCCUGCUGAUGCCUAUCCUUACC |
| | | CUGACACGCGCUCUGACUUCUCACACCGUGAUGCCUCUCUCCGCC |
| | | CCAACCCUCGUGCCACAGGAGCAUUAUGUGCGGGCAUCAGCACUG |
| | | UGGGAAAUCCAGCAAGUGGUGGAUGCCGACAGCAAGAUCGUGCAG |
| | | CUGUCCGAAAUUAGCAUGGAUAACUCACCCAAUCUGGCCUGGCCC |
| | | CUGAUUACCGUGGAGGAGCUGAAGAAGCUGGUGGAGCAGUGGAAU |
| | | CUGGUGAUCGGGUUCCUGUUUUCUCCUAGGUGGUAUUUUUACUAU |
| | | CUGGGCACCGGCCCCGAGGCCGGCCUGCCUUACGGAGCCAUCAGC |
| | | CGCCAGCGCCUGACUAAGUAUACCAUGGCCGAUCUGGUGUACGCC |
| | | CUGAGACACUUCUCAUUCAGCGGAUAUCUGAAGGUGACCGACAAC |
| | | GUGUACAUCAAAAACGCUGAUAUCGUGGAGGCCCCUACCAAGGUG |
| | | ACCUUCGGAGACGAUACAGUGAUCGAAGUGCAGGGGUACAAGUCC |
| | | GUGACCGAUUACAAGCACUGGCCGCAGAUCGCUCAGUUCGCCCCC |
| | | AGCGCCAGCGCCUUCUUUGGGAAGCUGUUCGCCGCCGAGACCCUG |
| | | AAGGCCACAGAAGAGACUUUUAAGCUUUCUUAUGGGAUUAAAAAC |
| | | CUGAGCGACCGGGUGGUGUUUGUGCUGUGGGCCCACGGCUUCGAG |
| | | CUGACCUCUAUGAAACACUAGGUGUACAUUGGAGACCCCGCCCAG |
| | | CUGCCUGCCCCCCGCACCUUGGUGACCAAGAGCAGCGACUACACC |
| | | AUUGUGCUGCCAGUGAUAGGGGCCAUUGUGGUGGGACUCUGCCUU |
| | | AUGGGCAUGGGCGUGUACAAGAUCCGGCUGCGCUGCCAGUCUAGC |
| | | GGAUACCAGAGAAUCUGA |
| 110 | SARS- | AUGGCUGCUCCUGGAUCUGCUAGAAGGCCUCUGCUGCUGCUUCUG |
| | CoV- | UUGCUCCUGCUGCUGGGCCUGAUGCAUUGUGCCUCUGCCGCCAUG |
| | 2_concat_ | UUCCACCUGGUGGACUUCCAAGUGACAAUCGCCGAGAUCCUGCUG |
| | 2.2_.1_D | AUCAUCAUGCGGACCGGCACACUGAUGAUCGAGAGAUUCGUGUCC |
| | CL_opt1 | CUGGCUAUCGACGCCUAUCCUCUGACCAAGCACAAGAUCUUCGUG |
| | | GACGGCGUGCCCUUCGUGGUGUCUACCGGCUACCACUUCAGAGAA |
| | | CUGGGAAUGAUGAUCUCCGCCGGCUUCAGCCUGUGGGUGUACAAG |
| | | CAGUUCGACACCUACAACCUGUGGGGCCUCGUGGCCGAAUGGUUC |
| | | CUGGCCUACAUCCUGUUCACCCGGUUCUUCUACGUGCUGGGCUCU |
| | | GCCGGCUUUCCCUUCAACAAAUGGGGCAAAGCCCGGCUGUACUAC |
| | | GACAGCAUGAGCUACUGUGCCGCCGGAACCACACAGACGGCCUGC |
| | | ACAGAUGAUAACGCCCUGGCCUAUUACAACACCGCCAGCAAGAUC |
| | | CUGGGCCUGCCUACACAGACAGUGGAUAGCUCUCAGGGCAGCGAG |
| | | UACGACGACAUCAAGGACCUCCCUAAAGAAAUCACCGUGGCCACC |
| | | AGCAGAACCCUGAGCUACUAUAAGGCCCUGCUGGCCGACAAGUUC |
| | | CCCGUGCUGCACGAUAUCGGCAACCCCAAGGCCAUCAAGUGCGAG |
| | | CUGUUCGAGAACAAGACCACACUGCCCGUGAACGUGGCCUUUGAG |
| | | CUGUGGGCUAAACGGGGCGUGUACAGCGUGAUCUACCUGUACCUG |
| | | ACCUUCUACCUGACCAACGACGUGUCCUUCCUGCACACCGACUUC |
| | | AGGAGCGAGAUCAUCGGCUACAAGGCCAUUGAUGGGGGCGUGACC |
| | | AGAGACAGCAGCGACUACACUAUCGUGCUGCCUGUGAUCGGCGCC |
| | | AUCGUUGUGGGACUGUGUCUGAUGGGCAUGGGAGUGUACAAGAUC |
| | | CGGCUGAGAUGCCAGAGCAGCGGCUACCAGAGAAUCUGA |
| 111 | SARS- | AUGGCCGCCCCCGGCAGCGCCCGCCGCCCCCUGCUGCUGCUGCUG |
| | CoV- | CUGCUGCUGCUGCUGGGCCUGAUGCACUGCGCCAGCGCCGCCAUG |
| | 2_concat_ | UUCCACCUGGUGGACUUCCAGGUGACCAUCGCCGAGAUCCUGCUG |
| | 2.2_.1_D | AUCAUCAUGCGCACCGGCACCCUGAUGAUCGAGCGCUUCGUGAGC |
| | CL_opt2 | CUGGCCAUCGACGCCUACCCCCUGACCAAGCACAAGAUCUUCGUG |
| | | GACGGCGUGCCCUUCGUGGUGAGCACCGGCUACCACUUCCGCGAG |
| | | CUGGGCAUGAUGAUCAGCGCCGGCUUCAGCCUGUGGGUGUACAAG |
| | | CAGUUCGACACCUACAACCUGUGGGGCCUGGUGGCCGAGUGGUUC |
| | | CUGGCCUACAUCCUGUUCACCCGCUUCUUCUACGUGCUGGGCAGC |
| | | GCCGGCUUCCCCUUCAACAAGUGGGGCAAGGCCCGCCUGUACUAC |
| | | GACAGCAUGAGCUACUGCGCCGCCGGCACCACCCAGACCGCCUGC |
| | | ACCGACGACAACGCCCUGGCCUACUACAACACCGCCAGCAAGAUC |
| | | CUGGGCCUGCCCACCCAGACCGUGGACAGCAGCCAGGGCAGCGAG |
| | | UACGACGACAUCAAGGACCUCCCCAAGGAGAUCACCGUGGCCACC |
| | | AGCCGCACCCUGAGCUACUACAAGGCCCUGCUGGCCGACAAGUUC |
| | | CCCGUGCUGCACGACAUCGGCAACCCCAAGGCCAUCAAGUGCGAG |
| | | CUGUUCGAGAACAAGACCACCCUGCCCGUGAACGUGGCCUUCGAG |
| | | CUGUGGGCCAAGCGCGGCGUGUACAGCGUGAUCUACCUGUACCUG |
| | | ACCUUCUACCUGACCAACGACGUGAGCUUCCUGGACACCGACUUC |
| | | AGCAGCGAGAUCAUCGGCUACAAGGCCAUCGACGGGGGCGUGACC |
| | | CGCGACAGCAGCGACUACACCAUCGUGCUGCCCGUGAUCGGCGCC |
| | | AUCGUGGUGGGCCUGUGCCUGAUGGGCAUGGGGGUGUACAAGAUC |
| | | CGCCUGCGCUGCCAGAGCAGCGGCUACCAGCGCAUCUAA |
| 112 | SARS- | AUGGCCGCUCCAGGCAGCGCCCGGCGCCCUCUGCUGUUGCUGCUG |
| | CoV- | CUGCUGCUGCUGCUGGGCCUGAUGCAUUGCGCCUCUGCCGCCAUG |
| | 2_concat_ | UUCCACCUGGUGGACUUCCAGGUGACCAUUGCCGAGAUUCUGCUG |
| | 2.2_.1 | AUUAUCAUGAGGACCGGCACACUGAUGAUCGAGAGAUUCGUCUCU |
| | D | CUGGCCAUCGACGCUUACCCUCUGACAAAACACAAGAUCUUCGUG |
| | CL_opt3 | GAUGGGGUGCCCUUCGUGGUGUCUACGGGGUACCACUUCAGGGAG |
| | | CUGGGAAUGAUGAUCUCCGCCGGCUUCAGCCUGUGGGUCUACAAA |
| | | CAGUUGGACACAUACAACCUGUGGGGCCUGGUGGCAGAGUGGUUU |
| | | CUCGCCUACAUCCUGUUCACCAGAUUCUUCUAUGUGCUGGGCUCA |
| | | GCUGGCUUUCCAUUCAAUAAAUGGGGCAAAGCCCGGCUGUAUUAC |
| | | GACAGCAUGAGCUACUGCGCUGCCGGAACCACACAGAGAGCCUGC |
| | | ACCGACGAUAAUGCCCUGGCCUACUACAACACCGCUAGCAAGAUU |
| | | CUGGGCCUGCCUACACAGACUGUGGAUAGGAGCCAGGGGAGCGAG |
| | | UACGACGACAUCAAGGAUCUGCCUAAGGAGAUCACAGUGGCCACC |
| | | AGGAGGACCCUGAGCUACUACAAGGCCCUGCUGGCCGAUAAGUUU |
| | | CCAGUGCUGCACGACAUUGGCAACCCUAAGGCCAUCAAGUGUGAA |
| | | CUGUUCGAAAACAAGACCACCCUGCCCGUGAACGUGGCCUUUGAG |
| | | CUGUGGGCUAAGCGCGGCGUGUACUCCGUGAUCUAUCUCUAUCUG |
| | | ACCUUUUACCUGACCAAUGACGUGAGCUUCCUGCACACCGACUUU |
| | | UCCUCCGAGAUCAUUGGCUACAAAGCCAUCGAUGGCGGCGUCACC |
| | | AGGGACAGCUCUGAUUACACUAUCGUGGUGCCUGUGAUCGGCGCU |
| | | AUCGUGGUGGGCCUGUGUUUGAUGGGCAUGGGAGUGUAUAAGAUU |
| | | AGGCUUAGGUGUCAGUCCAGCGGCUACCAGAGAAUCUAA |
| 113 | SARS- | AUGGCUGCUCCUGGAUCUGCUAGAAGGCCUCUGCUGCUGCUUCUG |
| | CoV- | UUGCUCCUGCUGCUGGGCCUGAUGCACUGUGCCUCUGCAGCCCUG |
| | 2_concat_ | UGUACCUUCCUGCUGAACAAAGAGAUGUACCUGAAGCUGCGGAGC |
| | 2.2_.2 | GACGUGCUGCUGCCCAUGUACAGCUUUGUGUCCGAGGAAACCGGC |
| | D | ACACUGAUCGUGAACUCUGUCCUGCUGUUCCUGGUGCCUGUGGUG |
| | CL_opt1 | GACAGCUACUACAGCCUGCUGAUGCCCAUCCUGACACUGACAAGA |
| | | GCCCUGACCAGCCACACCGUGAUGCCUCUGUCUGCUCCUACACUG |
| | | GUGCCCCAAGAGCACUAUGUGCGGGCAUCUGCCCUGUGGGAGAUC |
| | | CAGCAAGUGGUGGACGCCGACAGGAAGAUCGUGGAGCUGAGCGAG |
| | | AUCAGCAUGGACAACAGCCCCAAUCUGGCCUGGCCUCUGAUCACC |
| | | GUGGAAGAACUGAAGAAACUGCUGGAAGAGUGGAACCUCGUGAUC |
| | | GGCUUCCUGUUCAGCCCCAGAUGGUACUUCUACUACCUCGGCACA |
| | | GGACCCGAAGCCGGACUUCCUUAUGGCGCUAUCUCCCGGCAGCGG |
| | | CUGACCAAGUAUACCAUGGCCGAUCUGGUGUACGCCCUGCGGGAC |
| | | UUUAGCUUCAGCGGCUAUCUGAAGCUGACCGACAACGUGUACAUC |
| | | AAGAACGCCGACAUCGUGGAAGCCCCUACCAAAGUGACCUUCGGC |
| | | GACGAUACCGUGAUCGAGGUGCAGGGCUACAAGAGCGUGACCGAC |
| | | UACAAGCACUGGCCCCAGAUCGCCCAGUUUGCCCCUUCUGCCUCU |
| | | GCCUUCUUCGGCAAGCUGUUUGCCGCCGAGACACUGAAGGCCACC |
| | | GAAGAGACAUUCAAGCUGAGCUACGGCAUCAAGAAUCUGAGCGAG |
| | | AGAGUGGUGUUCGUGCUGUGGGCCCAUGGCUUUGAGCUGACCAGC |
| | | AUGAAGCACUACGUCUACAUCGGCGACCCCGCACAACUGCCCGCU |
| | | CCUAGAACACUGCUGACCAAAAGCAGCGACUACACCAUCGUGCUG |
| | | CCUGUGAUCGGCGCCAUCGUUGUGGGACUGUGUCUGAUGGGCAUG |
| | | GGCGUGUACAAGAUCCGGCUGAGAUGCCAGUCUAGCGGCUACCAG |
| | | AGAAUCUGA |
| 114 | SARS- | AUGGCCGCCCCCGGGAGCGCCCGCCGCCCCCUGCUGCUGCUGCUG |
| | CoV- | CUGCUGCUGCUGCUGGGCCUGAUGCACUGGGCCAGCGCCGCCCUG |
| | 2_concat_ | UGCACCUUCCUGCUGAACAAGGAGAUGUACCUGAAGCUGCGCAGC |
| | 2.2_.2_D | GACGUGCUGCUGCCCAUGUACAGCUUCGUGAGCGAGGAGACCGGC |
| | CL_opt2 | ACCCUGAUCGUGAACAGCGUGCUGCUGUUCCUGGUGCCCGUGGUG |
| | | GACAGCUAGUACAGCCUGCUGAUGCCCAUCCUGACCCUGACCCGC |
| | | GCCCUGACCAGCCACACCGUGAUGCCCCUGAGCGCCCCCACCCUG |
| | | GUGCCCCAGGAGCACUACGUGCGCGCCAGCGCCCUGUGGGAGAUC |
| | | CAGCAAGUGGUGGACGCCGACAGCAAGAUCGUGCAGCUGAGCGAG |
| | | AUCAGCAUGGACAACAGCCCCAACCUGGCCUGGCCCCUGAUCACC |
| | | GUGGAGGAGCUGAAGAAGGUGCUGGAGCAGUGGAACCUGGUGAUC |
| | | GGCUUCCUGUUCAGCCCCCGCUGGUACUUCUACUACCUGGGCACC |
| | | GGCCCCGAGGCCGGCCUGCCCUACGGCGCCAUCAGCCGCCAGCGC |
| | | CUGACCAAGUACACCAUGGCCGACCUGGUGUACGCCCUGCGCCAC |
| | | UUCAGCUUCAGCGGCUACCUGAAGCUGACCGACAACGUGUACAUC |
| | | AAGAACGCCGACAUCGUGGAGGCCCCCACCAAGGUGACCUUCGGC |
| | | GACGACACCGUGAUCGAGGUGGAGGGGUACAAGAGCGUGACCGAG |
| | | UAGAAGCACUGGCCCCAGAUCGCCCAGUUCGCCCCCAGGGGGAGC |
| | | GCCUUCUUCGGCAAGCUGUUCGCCGCCGAGACCCUGAAGGCCACC |
| | | GAGGAGACCUUCAAGCUGAGCUACGGCAUCAAGAACCUGAGCGAC |
| | | CGCGUGGUGUUCGUGCUGUGGGGCCACGGCUUCGAGCUGACCAGC |
| | | AUGAAGCACUACGUGUACAUGGGCGACCCCGCCCAGCUGCCCGCC |
| | | CCCCGCACCCUGCUGACCAAGAGCAGCGACUACACCAUCGUGCUG |
| | | CCCGUGAUCGGCGCCAUCGUGGUGGGCCUGUGCGUGAUGGGCAUG |
| | | GGCGUGUACAAGAUCCGCCUGCGCUGGCAGAGCAGCGGCUACCAG |
| | | CGCAUCUAA |
| 115 | SARS- | AUGGCCGCCCCAGGCUCUGCCAGAAGACCUCUGCUGCUGUUGCUG |
| | CoV- | CUGCUGCUGCUGGUGGGCCUGAUGCACUGCGCAAGUGCCGCCCUG |
| | 2_concat_ | UGUACUUUUCUGCUGAACAAAGAGAUGUAUCUGAAACUGAGAUCU |
| | 2.2_.2 | GACGUGCUGCUGCCAAUGUACUCUUUUGUGAGCGAGGAGACAGGA |
| | D | ACACUGAUCGUGAAUAGUGUGCUGCUGUUUCUGGUGCCCGUGGUG |
| | CL_opt3 | GACAGCUAUUACUCCCUCCUGAUGCCCAUCCUGAGACUGACCCGA |
| | | GCCCUCACCAGCCACACAGUGAUGCCUCUGUCUGCCCCCACCCUG |
| | | GUGCCACAGGAACAGUACGUGGGGGCCUCUGCACUGUGGGAGAUC |
| | | CAGCAAGUGGUGGAUGCCGAUAGGAAGAUUGUCCAGCUGUGAGAG |
| | | AUCAGCAUGGACAAUUCCCCUAAUCUGGCAUGGCCACUGAUCACA |
| | | GUGGAGGAGCUGAAAAAGCUGCUGGAGCAGUGGAACCUGGUGAUU |
| | | GGCUUCCUGUUCUCCCCCAGGUGGUAUUUCUAUUAUGUGGGGACC |
| | | GGCCCUGAGGCCGGCCUGCCCUAUGGCGCCAUUAGCAGGCAGAGG |
| | | CUGACCAAAUACACAAUGGCCGACCUGGUGUACGCUCUGAGACAU |
| | | UUUUCCUUCUCCGGGUACCUGAAGCUGACUGACAAUGUGUACAUU |
| | | AAGAACGCCGACAUCGUGGAGGCUCCUACCAAGGUCACAUUUGGA |
| | | GACGACACCGUGAUCGAGGUGCAGGGCUACAAGAGCGUGACAGAU |
| | | UACAAACACUGGCCCCAGAUCGCCCAGUUCGCCCCCAGCGCCUCC |
| | | GCCUUCUUUGGGAAGCUGUUCGCCGCUGAGACACUGAAGGCCACC |
| | | GAGGAGACCUUCAAACUGUCUUACGGGAUCAAGAAUCUGUCGGAU |
| | | AGGGUGGUGUUUGUGCUGUGGGCUCACGGGUUCGAACUGACAUCC |
| | | AUGAAGCACUACGUGUACAUAGGGGACCCCGCCGAGCUGCCCGCC |
| | | CCCAGGACGCUCCUGACCAAAUCCUCAGACUAUACCAUCGUGCUG |
| | | CCUGUGAUCGGGGCCAUUGUGGUGGGACUGUGCCUGAUGGGCAUG |
| | | GGCGUCUACAAGAUGCGGCUGAGAUGCGAGAGCUCCGGCUAUCAG |
| | | CGGAUCUGA |
| 116 | SARS- | AUGGCUGCUCCUGGAUCUGCUAGAAGGCCUCUGCUGCUGCUUCUG |
| | CoV- | UUGCUCCUGCUGCUGGGCCUGAUGCAUUGUGCCUCUGCAGCCUCU |
| | 2_concat_ | GCCGGCUUUCCCUUCAACAAAUGGGGCAAAGCCCGGCUGUACUAC |
| | 2.3_DC | GACAGCAUGAGCUACAACCUGAUCGACAGCUACUUCGUGGUCAAG |
| | L_opt1 | CGGCACACCUUCAGCAACUACCAGCACGAGGAACCCCAGAUCGCC |
| | | CAGUUUGCCCCUUCUGCCUCUGCCUUCUUCGGCAUGAGCAGAAUC |
| | | GGCAUGGACAUCAAGGACCUCCCUAAAGAAAUCACCGUGGCCACC |
| | | AGCAGAACCCUGAGCUACUACAAGCUGAUCAUCAACCUGGUGCAG |
| | | AUGGCCCCUAUCAGCGCCAUGGUCCGAAUGUACAUCUUCUUUCUG |
| | | UGCACCUUCCUGCUGAACAAAGAGAUGUACCUGAAGCUGCGGAGC |
| | | GACGUGCUGCUGCCUGCCUCUAAGAUUCUGGGCCUGCCUACACAG |
| | | ACCGUGGAUAGCUCUCAGGGCAGCGAGUACGACAUCAGCAGACAG |
| | | CGGCUGACCAAGUACACCAUGGCCGAUCUGGUGUACGCCCUGAGA |
| | | CACUUCGGCACCCUGAUGAUCGAGAGAUUCGUGUCCCUGGCUAUC |
| | | GACGCCUAUCCUCUGACCAAGCACCUGACACUGGGCGUGUACGAC |
| | | UACCUGGUGUCUACCCAAGAGUUCCGGUACAUGAACAGCCAGGGC |
| | | CUGCUCAUGUACAGCUUCGUGUCCGAGGAAACCGGCACACUGAUC |
| | | GUGAACUCUGUGCUGCUGUUCCUGGGCCUCGUGGAAGUGGAAAAA |
| | | GGCGUGCUGCCUCAGCUGGAACAGCCCUACGUGUUCAUCAAGCAG |
| | | UACAUCCGGAAGCUGCACGACGAGCUGACAGGCCACAUGCUGGAC |
| | | AUGUACUCCGUGAUGUAUCUGAACACACUGACCCUGGCCGUGCCU |
| | | UACAACAUGAGAGUGAUCCACUUCGGAGCCGGCGAGCUGUUCGAG |
| | | AACAAGACCACACUGCCCGUGAACGUGGCCUUUGAGCUGUGGGCC |
| | | AAGAGAAAGCUGUUCGCCGCCGAGACACUGAAGGCCACCGAAGAG |
| | | ACAUUCAAGGUGAGCUACGGCAUCUGCAGCGUGAUCGACCUCCUG |
| | | CUCGACGACUUCGUGGAAAUCAUCAAGAGCCAGGACCUGAGCCAG |
| | | GUGGUGUCCGACAUCGAUUACGUGCCCCUGAAGUCCGCCACCUGU |
| | | AUCACCCGGUGCAACAAGAUCUUCGUGGACGGCGUGCCCUUCGUG |
| | | GUGUCCACAGGCUACCACUUUAGAGAGCUGGGCGAGCUGAAAGUG |
| | | ACCUUCUUUCCCGACCUGAACGGCGACGUGGUGGCCAUCGAUUAU |
| | | AAGCACAGCAGGGACUACACCAUCGUGCUGCCAGUGAUCGGCGCC |
| | | AUCGUUGUGGGACUGUGUCUGAUGGGCAUGGGAGUGUACAAGAUC |
| | | CGGCUGAGAUGCCAGUCCAGCGGCUACCAGAGAAUCUGA |
| 117 | SARS- | AUGGCCGCCCCCGGCAGCGCCCGCCGCCCCCUGCUGCUGCUGCUG |
| | CoV- | CUGCUGCUGCUGCUGGGCCUGAUGCACUGCGCCAGCGCCGCCAGC |
| | 2_ | GCCGGCUUCCCCUUCAACAAGUGGGGCAAGGCCCGCCUGUACUAC |
| | concat_ | GACAGCAUGAGCUACAACCUGAUCGACAGCUACUUCGUGGUGAAG |
| | 2.3_DC | CGCCACACCUUCAGCAACUACCAGCACGAGGAGCCCCAGAUCGCC |
| | L_opt2 | CAGUUCGCCCCCAGCGCCAGCGCCUUCUUCGGCAUGAGCCGCAUC |
| | | GGCAUGGACAUCAAGGACCUCCCCAAGGAGAUCACCGUGGCCACC |
| | | AGCCGCACCCUGAGCUAGUACAAGCUGAUCAUCAACCUGGUGCAG |
| | | AUGGCCCCCAUCAGCGCCAUGGUGCGCAUGUACAUCUUCUUCCUG |
| | | UGCACCUUCCUGCUGAACAAGGAGAUGUACCUGAAGCUGCGCAGC |
| | | GACGUGCUGCUGCCCGCCAGCAAGAUCCUGGGCCUGCCCACCCAG |
| | | ACCGUGGACAGCAGCCAGGGCAGCGAGUACGACAUCAGCCGCCAG |
| | | CGCCUGACCAAGUACACCAUGGCCGACCUGGUGUACGCCCUGCGC |
| | | CACUUCGGCACCCUGAUGAUCGAGCGCUUCGUGAGCCUGGCCAUC |
| | | GACGCCUACCCCCUGACCAAGCACCUGACCCUGGGCGUGUACGAC |
| | | UACCUGGUGAGCACCCAGGAGUUCCGCUACAUGAACAGCCAGGGC |
| | | CUGCUGAUGUACAGCUUCGUGAGCGAGGAGACCGGCACCCUGAUC |
| | | GUGAACAGCGUGCUGCUGUUCCUGGGCCUGGUGGAGGUGGAGAAG |
| | | GGCGUGCUGCCCCAGCUGGAGCAGCCCUACGUGUUCAUCAAGCAG |
| | | UACAUCCGCAAGCUGCACGACGAGCUGACCGGCCACAUGCUGGAC |
| | | AUGUACAGCGUGAUGUACCUGAACACCCUGACCCUGGCCGUGCCC |
| | | UACAACAUGCGCGUGAUCCACUUCGGCGCCGGCGAGCUGUUCGAG |
| | | AACAAGACCACCCUGCCCGUGAACGUGGCCUUCGAGCUGUGGGCC |
| | | AAGCGCAAGCUGUUCGCCGCCGAGACCCUGAAGGCCACCGAGGAG |
| | | ACCUUCAAGCUGAGCUACGGCAUCUGCAGCGUGAUGGACCUCCUG |
| | | CUGGACGACUUCGUGGAGAUCAUCAAGAGCCAGGACCUGAGCCAG |
| | | GUGGUGAGCGACAUCGACUACGUGCCCCUGAAGAGCGCCACCUGU |
| | | AUCACCCGCUGCAACAAGAUCUUCGUGGACGGCGUGCCCUUCGUG |
| | | GUGAGCACCGGCUACCACUUCCGCGAGCUGGGCGAGGUGAAGGUG |
| | | ACCUUCUUCCCCGACCUGAAGGGCGACGUGGUGGCCAUCGACUAC |
| | | AAGCACAGCAGCGACUACACCAUCGUGCUGCCCGUGAUCGGCGCC |
| | | AUCGUGGUGGGCCUGUGCCUGAUGGGCAUGGGCGUGUACAAGAUC |
| | | CGCCUGCGCUGCCAGAGCAGCGGCUACCAGCGCAUCUAA |
| 118 | SARS- | AUGGCCGCCCCUGGCUCCGCCAGACGGCCACUGCUUCUCUUGCUG |
| | CoV- | CUGCUGCUGCUGCUGGGGCUGAUGCACUGUGCCUCCGCCGCCAGC |
| | 2_concat_ | GCCGGCUUCCCCUUUAACAAGUGGGGCAAGGCCAGGCUGUACUAU |
| | 2.3_DC | GAUUCCAUGAGCUACAACCUGAUCGACUCCUACUUCGUGGUGAAA |
| | L_opt3 | AGACACACCUUCAGCAAUUACCAGCACGAAGAGCCUCAGAUCGCU |
| | | CAGUUCGCCCCAAGCGCCUCUGCAUUCUUCGGGAUGUCCCGCAUC |
| | | GGCAUGGAUAUCAAGGAUCUGCCCAAGGAAAUCACCGUGGCCACC |
| | | AGUAGGACCCUGAGCUACUAUAAGCUGAUCAUCAACCUGGUGCAG |
| | | AUGGCCCCUAUCAGCGCUAUGGUGCGGAUGUACAUUUUUUUCCUG |
| | | UGCACCUUUCUGCUAAACAAGGAGAUGUACCUGAAGCUGAGAAGC |
| | | GAUGUGCUGCUGCCUGCCAGCAAAAUUCUGGGACUUCCCACCCAG |
| | | ACCGUGGAUAGCAGCCAGGGCAGCGAGUACGAUAUUUCCAGGCAG |
| | | AGACUGACAAAAUACACCAUGGCCGACCUGGUGUACGCUCUGAGG |
| | | CACUUCGGAACUCUGAUGAUCGAGAGGUUUGUGAGCCUGGCCAUC |
| | | GACGCCUACCCUCUGACCAAGCACCUGACCCUGGGCGUGUAUGAC |
| | | UACCUCGUGUCCACACAGGAGUUUCGCUACAUGAAUAGCCAGGGG |
| | | CUGCUUAUGUACUCCUUCGUGAGCGAGGAGACCGGAACCCUGAUC |
| | | GUGAAUUCCGUGCUGCUGUUCCUGGGCCUGGUGGAAGUGGAGAAG |
| | | GGAGUGCUGCCCCAGGUGGAGCAGCCCUAUGUGUUCAUCAAACAG |
| | | UAUAUCCGGAAGCUGCACGACGAACUGACCGGCCACAUGCUGGAU |
| | | AUGUACAGCGUCAUGUACCUCAACACUCUGACACUGGCCGUGCCC |
| | | UAUAAUAUGCGGGUUAUUCACUUCGGGGCUGGGGAACUGUUCGAG |
| | | AACAAGACCACACUGCCCGUGAAUGUGGCAUUCGAACUGUGGGCC |
| | | AAAAGGAAACUGUUUGCCGCUGAGACCCUGAAGGCCACUGAAGAG |
| | | ACCUUCAAACUGAGCUACGGGAUCUGCUCCGUGAUUGACCUCCUG |
| | | CUGGACGACUUCGUGGAAAUCAUCAAGAGCCAGGACCUGAGCCAG |
| | | GUGGUGAGCGAUAUCGAGUACGUGCCUCUCAAGUCCGCCACAUGU |
| | | AUCACCAGAUGCAAUJAAGAUCUUCGUGGACGGGGUGCCGUUCGU |
| | | GGUGUCCACCGGCUAUCAUUUUCGGGAGCUGGGGGAACUGAAGGU |
| | | GACAUUUUUCCCAGAUCUCAAUGGGGACGUGGUGGCUAUCGACUA |
| | | CAAGCACUCCAGCGACUACACCAUUGUGCUGCCCGUGAUCGGCGC |
| | | CAUCGUGGUCGGCCUGUGUCUGAUGGGGAUGGGGGUGUAUAAGAU |
| | | CCGGCUGCGGUGCCAGUCAAGCGGAUAUCAGCGCAUUUGA |
| 119 | SARS- | AUGGCUGCUCCUGGAUCUGCUAGAAGGCCUCUGCUGCUGCUUCUG |
| | CoV- | UUGCUCCUGCUGCUGGGCCUGAUGCACUGUGCCUCUGCAGCUCUG |
| | 2_concat_ | ACACUGGGGGUGUACGACUACCUGGUGUCUACCGAAGAGUUCCGG |
| | 2.4_DC | UACAUGAAGAGCCAGGGCCUGGUGGCCUCUAAGAUCCUGGGACUG |
| | L_opt1 | CCUACACAGACCGUGGAUAGCUCUCAGGGCAGCGAGUACGACUGC |
| | | AGCGUGAUCGAUCUGCUGCUCGACGACUUCGUGGAAAUCAUCAAG |
| | | AGCCAGGACCUGAGCCAGUACAUCCGGAAGCUGCACGAUGAGCUG |
| | | ACCGGCCAGAUGCUGGACAUGUACAGCGUGAUGCUGACCCCUGUG |
| | | UACAGCUUCCUGCCUGGCGUGUACUCCGUGAUCUACCUGUACCUG |
| | | ACCUUCAACCUGAUCGACAGCUACUUCGUGGUCAAGCGGCACACC |
| | | UUCAGCAACUACCAGCACGAGGAAAGCGCCGGCUUUCCCUUCAAC |
| | | AAAUGGGGCAAAGCCCGGCUGUACUACGACAGCAUGAGCUACUAC |
| | | CUGAACACACUGACCCUGGCCGUGCCUUACAACAUGAGAGUGAUC |
| | | CACUUCGGCGCUGGCCACAUGGAACUGCCAACAGGUGUUCACGCC |
| | | GGCACCGACCUGGAAGGCAAUUUCUAUGGACCCCAGGUGGUGUCC |
| | | GACAUCGACUACGUGCCACUGAAGUCCGCCACCUGUAUCACCAGA |
| | | UGCAACCACCACAGCAUCGGCUUCGAUUACGUGUACAACCCCUUC |
| | | AUGAUCGACGUGCAGCAGUGGGGACCUCAGAUCGCCCAGUUUGCC |
| | | CCUUCUGCCAGCGCCUUCUUUGGCAUGAGCCGGAUCGGCAUGAAG |
| | | CUGUUCGCCGCCGAAACACUGAAGGCCACCGAGGAAACCUUCAAG |
| | | CUGAGCUACGGCAUCGGCGUGUCCAUCUGCAGCACCAUGACCAAC |
| | | AGACAGUUCCACCAGAAGCUGCUGAAGUCUAUCCUGUGCACCUUC |
| | | CUGCUGAACAAAGAGAUGUACCUGAAGCUGCGGAGCGACGUGCUG |
| | | CUGCCUAUGUACUCCUUCGUGUCCGAAGAGACAGGCACCCUGAUC |
| | | GUGAACUCUGUGCUGCUGUUCCUGGGCACACUGAUGAUCGAGAGA |
| | | UUCGUGUCCCUGGCUAUCGACGCCUAUCCUCUGACCAAGCACGAG |
| | | CUGUUCGAGAACAAGACCACACUGCCCGUGAACGUGGCCUUUGAG |
| | | CUGUGGGCCAAGAGAGGCUGGGACUACCCCAAGUGCGAUCGGGCC |
| | | AUGCCUAACAUGCUGCGGAUUAUGGCCAGCCUGAUCAGCCGGCAG |
| | | AGACUGACCAAGUACACCAUGGCCGAUCUGGUGUACGCCCUGAGA |
| | | CACUUCUUCGCCUACGCCAACCGGAACAGAUUCCUGUAUAUUAUC |
| | | AAGCUGAUCUUCCUGUGGCUGCUGGCCGCCAUGCAGCGGAAGCUU |
| | | GAGAAGAUGGCUGACCAGGCUAUGACCCAGAUGUACAAGCAGCUG |
| | | AUCAUCAACCUGGUGCAGAUGGCCCCUAUCAGCGCCAUGGUUCGA |
| | | AUGUACAUCUUCUUCGACAUCAAGGACCUCCCGAAAGAAAUCACC |
| | | GUGGCCACCAGCAGAACCCUGUCCUACUACAAGAAAAUCUUCGUG |
| | | GACGGCGUGCCCUUCGUGGUGUCCACAGGCUACCACUUUAGAGAG |
| | | CUGGGCACAAGCAGCGGCGACGCCACAACAGCCUAUGCCAACUCC |
| | | GUGUUCAACAUCUGCCAGGCCGUGGUGUUCGACGAGAUCAGCAUG |
| | | GCCACCAACUACGACCUGAGCGUGGUCAAUGCCCGGCUGCGGGAA |
| | | CUGAAAGUGACCUUCUUUCCCGACCUGAACGGCGACGUGGUGGCA |
| | | AUCGAUUACAAGCACGGACUGGUGGAAGUGGAAAAGGGCGUGCUG |
| | | CCUCAGCUGGAACAGCCCUACGUGUUCAUCAAGUCCAGCGACUAC |
| | | ACCAUCGUGCUGCCAGUGAUCGGCGCCAUCGUUGUGGGACUGUGU |
| | | CUGAUGGGCAUGGGAGUGUACAAGAUCCGGCUGAGAUGCCAGAGC |
| | | AGCGGCUACCAGAGAAUCUGA |
| 120 | SARS- | AUGGCUGCUCCUGGAUCUGCUAGAAGGCCUCUGCUGCUGCUUCUG |
| | CoV- | UUGCUCCUGCUGGUGGGCCUGAUGCACUGUGCCUCUGCAGCUCUG |
| | 2_concat_ | ACACUGGGCGUGUACGACUACCUGGUGUCUACCCAAGAGUUCCGG |
| | 2.4_DC | UACAUGAACAGCCAGGGCCUGCUGGCCUCUAAGAUCCUGGGACUG |
| | L_opt2 | CCUACACAGACCGUGGAUAGCUCUCAGGGCAGCGAGUACGACUGC |
| | | AGCGUGAUCGAUCUGCUGCUCGACGACUUCGUGGAAAUCAUCAAG |
| | | AGCCAGGACCUGAGCCAGUACAUCCGGAAGCUGCACGAUGAGCUG |
| | | ACCGGCCACAUGCUGGACAUGUACAGCGUGAUGCUGACCCCUGUG |
| | | UACAGCUUCCUGCCUGGCGUGUACUCCGUGAUCUACCUGUACCUG |
| | | ACCUUCAACCUGAUCGACAGCUACUUCGUGGUCAAGCGGCACACC |
| | | UUCAGCAACUACCAGCACGAGGAAAGCGCCGGCUUUCCCUUCAAC |
| | | AAAUGGGGCAAAGCCCGGCUGUACUACGACAGCAUGAGCUACUAC |
| | | CUGAACACACUGACCCUGGCCGUGCCUUACAACAUGAGAGUGAUC |
| | | CACUUCGGCGCUGGCCACAUGGAACUGCCAACAGGUGUUCACGCC |
| | | GGCACCGACCUGGAAGGCAAUUUCUAUGGACCCCAGGUGGUGUCC |
| | | GACAUCGACUACGUGCCACUGAAGUCCGCCACCUGUAUCACCAGA |
| | | UGCAACCACCACAGCAUCGGCUUCGAUUACGUGUACAACCCCUUC |
| | | AUGAUCGACGUGCAGCAGUGGGGACCUCAGAUCGCCCAGUUUGCC |
| | | CCUUCUGCCAGCGCCUUGUUUGGCAUGAGCCGGAUCGGCAUGAAG |
| | | CUGUUCGCCGCCGAAACAGUGAAGGCCACCGAGGAAACCUUCAAG |
| | | CUGAGCUACGGCAUCGGCGUGUCCAUCUGCAGCACCAUGACCAAC |
| | | AGACAGUUCCACCAGAAGCUGCUGAAGUCUAUCCUGUGCACCUUC |
| | | CUGCUGAACAAAGAGAUGUACCUGAAGCUGCGGAGCGACGUGCUG |
| | | CUGCCUAUGUACUCCUUCGUGUCCGAAGAGACAGGCACCCUGAUC |
| | | GUGAACUCUGUGCUGCUGUUCCUGGGCACACUGAUGAUGGAGAGA |
| | | UUCGUGUCCCUGGCUAUGGACGCCUAUCCUCUGACCAAGCACGAG |
| | | CUGUUCGAGAACAAGACCACACUGCCCGUGAACGUGGCCUUUGAG |
| | | CUGUGGGCCAAGAGAGGCUGGGACUACCCCAAGUGCGAUCGGGCC |
| | | AUGCCUAACAUGCUGCGGAUUAUGGCCAGCCUGAUCAGCCGGCAG |
| | | AGACUGACCAAGUACACCAUGGCCGAUCUGGUGUACGCCCUGAGA |
| | | CACUUCUUCGCCUACGCCAACCGGAACAGAUUCCUGUAUAUUAUC |
| | | AAGCUGAUCUUCCUGUGGGUGCUGGCCGCCAUGCAGCGGAAGCUU |
| | | GAGAAGAUGGCUGACCAGGCUAUGACCCAGAUGUACAAGCAGCUG |
| | | AUCAUCAACCUGGUGCAGAUGGCCCCUAUCAGCGCCAUGGUUCGA |
| | | AUGUACAUCUUCUUCGACAUCAAGGACCUCCCGAAAGAAAUCACC |
| | | GUGGCCACCAGCAGAACCGUGUCCUACUACAAGAAAAUCUUCGUG |
| | | GACGGCGUGCCCUUCGUGGUGUCCACAGGCUACCACUUUAGAGAG |
| | | CUGGGCACAAGCAGCGGCGACGCCACAACAGCCUAUGCCAACUCC |
| | | GUGUUCAACAUCUGCCAGGCCGUGGUGUUCGACGAGAUCAGCAUG |
| | | GCCACCAACUACGACCUGAGCGUGGUCAAUGCCCGGCUGCGGGAA |
| | | CUGAAAGUGACCUUCUUUCCCGACCUGAACGGCGACGUGGUGGCA |
| | | AUCGAUUACAAGCACGGACUGGUGGAAGUGGAAAAGGGCGUGCUG |
| | | CCUCAGCUGGAACAGCCCUACGUGUUCAUCAAGUCCAGCGACUAC |
| | | ACCAUCGUGCUGCCAGUGAUCGGCGCCAUCGUUGUGGGACUGUGU |
| | | CUGAUGGGCAUGGGAGUGUACAAGAUCCGGCUGAGAUGCCAGAGC |
| | | AGCGGCUACCAGAGAAUCUGA |
| 121 | SARS- | AUGGCCGCCCCAGGCUCUGCCAGGCGCCCCCUGCUGCUCCUGCUG |
| | CoV- | CUGCUCCUGCUGCUGGGCCUGAUGCACUGCGCUAGCGCAGCCCUG |
| | 2_concat_ | ACACUGGGCGUGUACGAUUACCUGGUGAGCACACAGGAGUUCCGG |
| | 2.4_DC | UACAUGAAUAGCCAGGGCCUGCUGGCCAGCAAAAUUUUGGGGCUG |
| | L_opt3 | CCGAGACAGACUGUGGACUCCUCCGAGGGCUCUGAAUACGAUUGG |
| | | AGGGUGAUCGACCUCCUGCUGGAUGACUUCGUGGAAAUCAUCAAG |
| | | UCUCAGGAUCUGUCCCAGUACAUUCGCAAACUGCACGAUGAGCUG |
| | | ACCGGACACAUGCUGGAUAUGUACAGCGUGAUGCUGACCCCUGUG |
| | | UACUCCUUCCUGCCUGGCGUCUACAGCGUGAUCUACCUGUAUCUG |
| | | ACUUUCAACCUGAUCGAUUCCUACUUCGUCGUGAAGCGCCACACU |
| | | UUUUCCAACUACCAGCACGAGGAGAGUGCCGGCUUCCCCUUCAAC |
| | | AAAUGGGGCAAGGCCAGACUGUAUUACGAUUCCAUGUCUUACUAC |
| | | CUGAAUACACUGACACUGGCCGUGCCCUACAACAUGAGAGUGAUC |
| | | CACUUUGGAGCCGGCCACAUGGAGCUGCCUACCGGGGUGCACGCC |
| | | GGGACCGACCUGGAAGGGAAUUUCUAUGGGCCUCAGGUGGUGUCU |
| | | GACAUUGACUAUGUGCCCCUGAAAUCCGCCACUUGCAUCACCCGG |
| | | UGCAACCAUCACUCUAUCGGCUUUGACUAUGUGUACAACCCAUUC |
| | | AUGAUUGACGUGCAGCAGUGGGGCCCACAGAUUGCCCAGUUCGCC |
| | | CCCAGCGCCAGCGCCUUCUUUGGCAUGAGCAGGAUCGGCAUGAAG |
| | | CUCUUUGCCGCCGAAACCCUGAAGGCAACCGAGGAAACCUUCAAA |
| | | CUGUCCUACGGUAUCGGAGUGAGCAUCUGCUCCACAAUGACCAAC |
| | | AGGCAGUUUCAUCAGAAGCUCCUGAAGUCCAUCCUGUGCACCUUC |
| | | CUGCUGAAUAAGGAGAUGUAUCUGAAGCUGAGAUCCGACGUGUUG |
| | | CUGCCUAUGUACUCCUUUGUGAGCGAGGAGACAGGAACUCUGAUC |
| | | GUGAACAGCGUGGUGCUGUUCCUGGGAACCCUGAUGAUCGAGAGG |
| | | UUCGUGAGCCUGGCCAUCGACGCCUACCCUCUGACCAAACACGAA |
| | | CUGUUCGAGAAUAAAACAACACUGCCAGUGAAUGUGGCCUUCGAA |
| | | CUGUGGGCUAAAAGAGGCUGGGAUUAUCCUAAAUGUGACCGGGCC |
| | | AUGCCUAACAUGCUGAGGAUUAUGGCUUCACUGAUCAGCAGGCAG |
| | | AGACUGACCAAGUACACCAUGGCCGACCUGGUGUAUGCCCUGAGG |
| | | CAUUUCUUCGCCUAUGCUAAUAGAAACAGGUUCCUGUACAUCAUU |
| | | AAGCUUAUCUUUUUGUGGCUGCUGGCCGCCAUGCAGCGGAAGCUU |
| | | GAGAAAAUGGCUGAUCAGGCCAUGACUCAGAUGUACAAGCAGCUG |
| | | AUCAUCAACCUGGUGCAGAUGGCCCCCAUUAGCGCCAUGGUGCGG |
| | | AUGUAUAUCUUCUUGGACAUCAAGGAUCUGCCAAAAGAGAUCACA |
| | | GUGGCCACAUCUCGUACGCUGAGCUACUACAAGAAGAUUUUCGUG |
| | | GACGGCGUGCCAUUCGUGGUGAGCACCGGCUACCACUUUAGAGAG |
| | | CUGGGCACCAGCUCUGGAGACGCUACGACCGCAUAGGCAAACAGC |
| | | GUGUUUAACAUCUGCCAGGCCGUGGUCUUCGAUGAGAUCAGCAUG |
| | | GCUACCAACUACGAUCUGAGUGUGGUUAACGCUCGCCUGCGCGAG |
| | | CUUAAGGUCACCUUCUUCCCAGACCUCAAGGGCGAUGUGGUCGCC |
| | | AUUGACUAGAAACACGGCCUGGUGGAGGUGGAGAAGGGGGUGCUG |
| | | CCACAGCUGGAGCAGCCCUACGUGUUCAUUAAGUCUAGCGAUUAC |
| | | ACAAUCGUGCUGCCCGUUAUCGGAGCUAUUGUGGUGGGCCUGUGC |
| | | CUGAUGGGCAUGGGAGUGUACAAGAUCAGGCUGCGGUGCCAGAGC |
| | | UCAGGGUACGAGAGGAUUUGA |
| 122 | SARS- | AUGGCUGCUCCUGGAUCUGCUAGAAGGCCUCUGCUGCUGCUUCUG |
| | CoV- | UUGCUCCUGCUGCUGGGCCUGAUGCACUGUGCCUCUGAGCUCUGA |
| | 2_concat_ | GACUGGGGGUGUACGACUACCUGGUGUCUACGGAAGAGUUCCGGU |
| | 2.4.1_D | AGAUGAAGAGGGAGGGGCUGCUGGCGUCUAAGAUCCUGGGACUGC |
| | CL_opt1 | CUACACAGACCGUGGAUAGCUCUCAGGGCAGCGAGUACGACUGCA |
| | | GCGUGAUCGAUCUGCUGCUCGACGAGUUCGUGGAAAUCAUGAAGA |
| | | GCCAGGACCUGAGCCAGUACAUCCGGAAGCUGCACGAUGAGCUGA |
| | | CCGGCCACAUGCUGGACAUGUACAGCGUGAUGCUGACCCCUGUGU |
| | | ACAGCUUCCUGCCUGGCGUGUACUCCGUGAUCUACCUGUACCUGA |
| | | CCUUCAACCUGAUCGACAGCUACUUCGUGGUCAAGCGGCACACCU |
| | | UCAGCAACUACCAGCACGAGGAAAGCGCCGGCUUUCCCUUCAACA |
| | | AAUGGGGCAAAGCCCGGCUGUACUACGACAGCAUGAGCUACUACC |
| | | UGAACACACUGACCCUGGCCGUGCCUUACAACAUGAGAGUGAUCC |
| | | ACUUCGGCGCUGGCCACAUGGAACUGCCAACAGGUGUUCACGCCG |
| | | GCACCGACCUGGAAGGCAAUUUCUAUGGACCCCAGGUGGUGUCCG |
| | | ACAUCGACUACGUGCCACUGAAGUCCGCCACCUGUAUCACCAGAU |
| | | GCAACCACCACAGCAUCGGCUUCGAUUACGUGUACAACCCCUUCA |
| | | UGAUCGACGUGCAGCAGUGGGGACCUCAGAUCGCCCAGUUUGCCC |
| | | CUUCUGCCAGCGCCUUCUUUGGCAUGAGCCGGAUCGGCAUGAAGC |
| | | UGUUCGCCGCCGAAACACUGAAGGCCACCGAGGAAACCUUCAAGC |
| | | UGAGCUACGGCAUCGGCGUGUCCAUCUGCAGCACCAUGACCAACA |
| | | GACAGUUCCACCAGAAGCUGCUGAAGUCCAUCAGCAGCGACUAGA |
| | | CCAUCGUGCUGCCUGUGAUCGGCGCCAUCGUUGUGGGACUGUGUC |
| | | UGAUGGGCAUGGGAGUGUACAAGAUCCGGCUGAGAUGCCAGAGCA |
| | | GCGGCUACCAGAGAAUCUGA |
| 123 | SARS- | AUGGCCGCCCCCGGCAGCGCCCGCCGCCCCCUGCUGCUGCUGCUG |
| | CoV- | CUGCUGCUGCUGCUGGGCCUGAUGCACUGGGCCAGCGCCGCCCUG |
| | 2_concat_ | ACCCUGGGCGUGUACGACUACCUGGUGAGCACCCAGGAGUUCCGC |
| | 2.4.1_D | UACAUGAACAGCCAGGGCCUGCUGGCCAGCAAGAUCCUGGGCCUG |
| | CL_opt2 | CCCACCCAGACCGUGGACAGCAGCCAGGGCAGCGAGUACGACUGC |
| | | AGCGUGAUCGACCUCCUGCUGGACGACUUCGUGGAGAUCAUCAAG |
| | | AGCCAGGACCUGAGCCAGUACAUCCGCAAGCUGCACGACGAGCUG |
| | | ACCGGCCACAUGCUGGACAUGUACAGCGUGAUGCUGACCCCCGUG |
| | | UACAGCUCCUGCCCGGCGUGUACAGCGUGAUGUACCUGUACCUGA |
| | | CCUUCAACCUGAUCGACAGCUACUUCGUGGUGAAGCGCCACACCU |
| | | UCAGCAACUACCAGCACGAGGAGAGCGCCGGCUUCCCCUUCAACA |
| | | AGUGGGGCAAGGCCCGCCUGUACUACGACAGCAUGAGCUACUACC |
| | | UGAACACCCUGACCCUGGCCGUGCCCUACAACAUGCGCGUGAUCC |
| | | ACUUCGGCGCCGGCCACAUGGAGCUGCCCACCGGCGUGCACGCCG |
| | | GCACCGACCUGGAGGGCAACUUCUACGGCCCCCAGGUGGUGAGCG |
| | | ACAUCGACUACGUGCCCCUGAAGAGCGCCACCUGUAUCACCCGCU |
| | | GCAACCACCACAGCAUCGGCUUCGACUACGUGUACAACCCCUUCA |
| | | UGAUCGACGUGCAGCAGUGGGGCCCCCAGAUCGCCCAGUUCGCCC |
| | | CCAGCGCCAGCGCCUUCUUCGGCAUGAGCCGCAUCGGCAUGAAGC |
| | | UGUUCGCCGCCGAGACCCUGAAGGCCACCGAGGAGACCUUCAAGC |
| | | UGAGCUACGGCAUCGGCGUGAGCAUCUGCAGCACCAUGACCAACC |
| | | GCCAGUUCCACCAGAAGCUGCUGAAGAGCAUCAGCAGCGACUACA |
| | | CCAUCGUGCUGCCCGUGAUCGGCGCCAUCGUGGUGGGCCUGUGCC |
| | | UGAUGGGCAUGGGCGUGUACAAGAUCCGCCUGCGCUGCCAGAGCA |
| | | GCGGCUACCAGCGCAUCUAA |
| 124 | SARS- | AUGGCAGCCCCUGGCUCCGCCAGAAGACCACUGCUGCUCCUGCUG |
| | CoV- | CUGCUGCUCCUGCUGGGCCUGAUGCACUGCGCCUCCGCUGCCCUG |
| | 2_concat_ | ACCCUGGGCGUGUAUGACUACCUGGUGAGCACCCAGGAGUUCAGA |
| | 2.4.1_D | UACAUGAACUCUCAGGGCCUCCUGGCCUCCAAGAUCCUGGGCCUG |
| | CL_opt3 | CCCACCCAAACCGUGGACUCAUCUCAGGGAUCCGAGUACGAUUGC |
| | | UCCGUGAUCGACCUCCUGCUGGACGAUUUCGUGGAAAUCAUUAAG |
| | | AGCCAGGAUCUGUCUCAGUAUAUCAGAAAGCUGCAUGACGAGCUG |
| | | ACCGGCCACAUGCUCGACAUGUAUUCCGUGAUGCUGACACCCGUG |
| | | UACUCUUUCCUGCCCGGCGUGUAUUCUGUGAUCUAUCUCUACCUG |
| | | ACCUUCAACCUGAUCGACUCAUACUUCGUGGUGAAGAGACAUACC |
| | | UUCAGCAACUAUCAGCACGAGGAGAGCGCUGGCUUCCCCUUUAAC |
| | | AAGUGGGGCAAGGCUAGACUGUAGUAUGAUAGCAUGUCUUAUUAC |
| | | CUGAACACACUGACCCUGGCUGUGCCAUACAACAUGAGGGUGAUU |
| | | CACUUUGGCGCUGGACACAUGGAGCUGCCUACCGGAGUGCACGCC |
| | | GGCACCGAUCUGGAGGGAAAUUUCUAUGGGCCCCAGGUGGUGUCC |
| | | GACAUCGACUACGUGCCCCUGAAGUCGGCCACAUGUAUUACUCGC |
| | | UGCAACCACCACAGCAUUGGCUUUGAUUAUGUGUAUAACCCCUUG |
| | | AUGAUCGACGUGCAGCAGUGGGGCCCACAGAUCGGUCAGUUCGCC |
| | | CCUUCAGCCAGUGCCUUCUUUGGGAUGUCCAGAAUCGGAAUGAAG |
| | | CUGUUUGCCGCCGAGACCCUGAAGGCCACAGAAGAGACCUUUAAG |
| | | CUGUCCUACGGAAUCGGCGUGAGCAUUUGCUCCACCAUGACCAAU |
| | | AGACAGUUCCACCAGAAACUGCUGAAGUCUAUUUCUAGCGACUAU |
| | | ACCAUUGUGCUGCCCGUGAUCGGGGCCAUCGUGGUGGGCCUGUGC |
| | | CUGAUGGGAAUGGGCGUGUACAAGAUUAGGCUGAGGUGUCAGAGC |
| | | AGCGGGUACCAGGGGAUUUAA |
| 125 | SARS- | AUGGCUGCUCCUGGAUCUGCUAGAAGGCCUCUGCUGCUGCUUCUG |
| | CoV- | UUGCUCCUGCUGGUGGGCCUGAUGCACUGUGCCUCUGCAGCCCUG |
| | 2 | UGUACCUUCCUGCUGAACAAAGAGAUGUACCUGAAGCUGCGGAGC |
| | concat_ | GACGUGCUGCUGCCCAUGUACAGCUUUGUGUCCGAGGAAACCGGC |
| | 2.4.2_D | ACACUGAUCGUGAACUCUGUGCUGCUGUUCCUGGGCACCCUGAUG |
| | CL_opt1 | AUCGAGAGAUUCGUGUCCCUGGCUAUCGACGCCUAUCCUCUGACC |
| | | AAGCACGAGCUGUUCGAGAACAAGACCACACUGCCCGUGAACGUG |
| | | GCCUUUGAGCUGUGGGCCAAGAGAGGCUGGGACUACCCCAAGUGC |
| | | GAUCGGGCCAUGCCUAACAUGCUGCGGAUUAUGGCCAGCCUGAUC |
| | | AGCCGGCAGAGACUGACCAAGUACACCAUGGCCGAUCUGGUGUAC |
| | | GCCCUGAGAGACUUCUUCGCGUACGCCAACCGGAACAGAUUCCUG |
| | | UAUAUUAUCAAGCUGAUCUUCCUGUGGCUGCUGGCCGCCAUGCAG |
| | | CGGAAGCUUGAGAAGAUGGCUGACCAGGCCAUGACACAGAUGUAC |
| | | AAGCAGCUGAUCAUCAACCUGGUGCAGAUGGCCCCUAUCAGCGCC |
| | | AUGGUCCGAAUGUACAUCUUCUUCGACAUCAAGGACCUCCCGAAA |
| | | GAAAUCACCGUGGCCACCAGCAGAACCCUGAGCUAGUACAAGAAA |
| | | AUCUUCGUGGACGGCGUGCCCUUCGUGGUGUCUACCGGCUACCAC |
| | | UUUAGAGAGCUGGGCACAAGCAGCGGCGACGCCACAACAGCCUAU |
| | | GCCAACAGCGUGUUCAACAUCUGCCAGGCCGUGGUGUUCGACGAG |
| | | AUCAGCAUGGCCACCAAGUACGACCUGAGCGUGGUCAAUGCCCGG |
| | | CUGCGGGAACUGAAAGUGACCUUCUUUCCCGACCUGAACGGCGAC |
| | | GUGGUGGCAAUCGAUUACAAGCACGGACUGGUGGAAGUGGAAAAG |
| | | GGCGUGCUGCCUCAGCUGGAAGAGCCCUACGUGUUCAUCAAGAGC |
| | | AGCGACUACACCAUCGUGCUGCCAGUGAUCGGCGCCAUCGUUGUG |
| | | GGACUGUGUCUGAUGGGCAUGGGCGUGUACAAGAUCCGGCUGAGA |
| | | UGCCAGAGCAGCGGCUAUCAGAGAAUCUGA |
| 126 | SARS- | AUGGCCGCCCCCGGCAGCGCCCGCCGCCCCCUGCUGCUGCUGCUG |
| | CoV- | CUGCUGCUGCUGCUGGGCCUGAUGCACUGCGCCAGCGCCGCCCUG |
| | 2_concat_ | UGCACCUUCCUGCUGAACAAGGAGAUGUACCUGAAGCUGCGCAGC |
| | 2.4.2_D | GACGUGCUGCUGCCCAUGUACAGCUUCGUGAGCGAGGAGACCGGC |
| | CL_opt2 | ACCCUGAUCGUGAACAGCGUGCUGCUGUUCCUGGGCACCCUGAUG |
| | | AUCGAGCGCUUCGUGAGCCUGGCCAUCGACGCCUACCCCCUGACC |
| | | AAGCACGAGCUGUUCGAGAACAAGACCACCCUGCCCGUGAACGUG |
| | | GCCUUCGAGCUGUGGGCCAAGCGCGGCUGGGACUACCCCAAGUGC |
| | | GACCGCGCCAUGCCCAACAUGCUGCGCAUCAUGGCCAGCCUGAUC |
| | | AGCCGCCAGCGCCUGACCAAGUACACCAUGGCCGACCUGGUGUAC |
| | | GCCCUGCGCCACUUCUUCGCCUACGCCAACCGCAACCGCUUCCUG |
| | | UACAUCAUCAAGCUGAUCUUCCUGUGGCUGCUGGCCGCCAUGCAG |
| | | CGGAAGCUGGAGAAGAUGGCCGACCAGGCCAUGACCCAGAUGUAC |
| | | AAGCAGCUGAUCAUCAACCUGGUGCAGAUGGCCCCCAUCAGCGCC |
| | | AUGGUGCGCAUGUACAUCUUCUUCGACAUCAAGGACCUCCCCAAG |
| | | GAGAUCACCGUGGCCACCAGCCGCACCCUGAGCUACUACAAGAAG |
| | | AUCUUCGUGGACGGCGUGCCCUUCGUGGUGAGCACCGGCUACCAC |
| | | UUCCGCGAGCUGGGCACCAGCAGCGGCGACGCCACCACCGCCUAC |
| | | GCCAACAGCGUGUUCAACAUCUGCCAGGCCGUGGUGUUCGACGAG |
| | | AUCAGCAUGGCCACCAACUACGACCUGAGCGUGGUGAACGCCCGC |
| | | CUGCGCGAGCUGAAGGUGACCUUCUUCCCCGACCUGAACGGCGAC |
| | | GUGGUGGCCAUCGACUACAAGCACGGCCUGGUGGAGGUGGAGAAG |
| | | GGCGUGCUGCCCCAGCUGGAGCAGCCCUACGUGUUCAUCAAGAGC |
| | | AGCGACUACACCAUCGUGCUGCCCGUGAUCGGCGCCAUCGUGGUG |
| | | GGCCUGUGCCUGAUGGGCAUGGGCGUGUACAAGAUCCGCCUGCGC |
| | | UGCCAGAGCAGCGGCUACCAGCGCAUCUAA |
| 127 | SARS- | AUGGCCGCCCCAGGCUCUGCCAGGGGGCCCCUGCUGCUCCUGCUG |
| | CoV- | CUGCUCCUGCUGCUGGGGGUGAUGGACUGGGCUAGCGCAGCCCUG |
| | 2_concat_ | UGUACAUUCCUGCUGAAUAAAGAGAUGUAUCUGAAGCUGAGAUCU |
| | 2.4.2_D | GACGUCCUGCUGCCCAUGUACAGCUUCGUGUCCGAGGAGACGGGG |
| | CL_opt3 | ACCCUGAUCGUGAAUUCUGUGCUCCUGUUCCUGGGCACCCUGAUG |
| | | AUUGAGAGAUUCGUGAGCCUGGCCAUCGAĢGCCUACCCUCUGAGG |
| | | AAAGACGAGGUGUUCGAGAAGAAGAçGACGCUGGCCGUGAACGUG |
| | | GĢCUUUGAGCUGUGGGCUAAGCGCGGAUGGGACUACCCCAAGUGC |
| | | GACCGCGCCAUGCCUAACAUGCUGAGAAUUAUGGCCAGCUUAAUC |
| | | AGCCGGCAGAGGCUGACAAAAUACACCAUGGCCGACCUCGUGUAC |
| | | GCCCUGGGGGACUUCUUCGCCUACGCAAACCGCAACCGCUUCCUG |
| | | UAGAUCAUCAAGCUGAUCUUUCUGUGGCUGCUGGCCGCCAUGCAG |
| | | CGGAAGCUGGAAAAGAUGGCCGACGAGGCCAUGACCCAGAUGUAC |
| | | AAGCAGCUGAUCAUCAAUCUGGUGCAGAUGGCCCCAAUCUCCGCC |
| | | AUGGUGCGAAUGUACAUUUUUUUCGAUAUUAAGGACCUCCCUAAG |
| | | GAGAUUACCGUGGCCACCAGCCGUACCCUGAGCUACUACAAAAAG |
| | | AUCUUCGUGGACGGCGUGCCCUUUGUGGUGAGCACCGGGUAUCAC |
| | | UUCAGGGAACUGGGUACCUCCUCCGGGGAUGCAACUACCGCCUAC |
| | | GCCAAUUCUGUGUUCAACAUUUGCCAGGCCGUGGUCUUCGAUGAG |
| | | AUCAGCAUGGCCACCAACUACGAUCUGUCUGUGGUGAACGCCCGC |
| | | CUGCGCGAGCUGAAAGUGACCUUCUUCCCUGACCUGAAUGGCGAC |
| | | GUGGUGGCUAUUGACUACAAACACGGCCUGGUGGAGGUGGAAAAA |
| | | GGAGUGCUGCCCCAGCUGGAGCAGCCAUACGUGUUCAUCAAAAGU |
| | | UCUGAUUACACCAUCGUGUUGCCAGUGAUUGGAGCUAUCGUGGUG |
| | | GGGCUGUGCCUGAUGGGGAUGGGCGUGUACAAGAUUAGACUGAGG |
| | | UGCCAGAGCUCUGGCUACCAGAGAAUCUGA |
| 128 | SARS- | AUGGCUGCUCCUGGAUCUGCUAGAAGGCCUCUGCUGCUGCUUCUG |
| | CoV- | UUGCUCCUGCUGGUGGGCCUGAUGCACUGUGCCUCUGCAGCCCUG |
| | 2_concat_ | UGUACCUUCCUGCUGAACAAAGAGAUGUACCUGAAGCUGCGGAGC |
| | 2.5_DC | GACGUGCUGCUGCCCAAGAUCUUUGUGGAUGGCGUGCCCUUCGUG |
| | L_opt1 | GUGUCCACCGGCUACCACUUUAGAGAGCUGGGCUCUGCCGGCUUU |
| | | CCCUUCAACAAAUGGGGCAAAGCCCGGCUGUACUACGACAGCAUG |
| | | AGCUACGACAUCAAGGACCUCCCUAAAGAAAUCACCGUGGCCACC |
| | | AGCAGAACCCUGAGCUACUACAAGGGCACCCUGAUGAUCGAGAGA |
| | | UUCGUGUCCCUGGCUAUCGACGCCUAUCCUCUGACCAAGGACGAG |
| | | CUGUUCGAGAACAAGACCACACUGCCCGUGAACGUGGCCUUUGAG |
| | | CUGUGGGCCAAGAGAGCCAGCAAGAUCCUGGGACUGCCUACACAG |
| | | ACCGUGGAUAGCUCUCAGGGCAGCGAGUACGACAAGGUGUUUGCC |
| | | GCCGAGACACUGAAGGCCACCGAGGAAACCUUCAAGCUGUCCUAC |
| | | GGCAUCAUCAGCAGACAGCGGCUGACCAAGUACACCAUGGCCGAU |
| | | CUGGUGUACGCCCUGCGGCACUUCAUGUACAGCUUCGUGUCCGAA |
| | | GAGACAGGCACACUGAUCGUGAACUCUGUGCUGCUGUUCCUGAGC |
| | | AGCGACUACACCAUCGUGCUGCCUGUGAUCGGCGCCAUCGUUGUG |
| | | GGACUGUGUCUGAUGGGCAUGGGGGUGUACAAGAUCCGGCUGAGA |
| | | UGCCAGAGCAGCGGCUAUCAGAGAAUCUGA |
| 129 | SARS- | AUGGCCGCCCCCGGCAGCGCCCGCCGCCCCCUGCUGCUGCUGCUG |
| | CoV- | CUGCUGCUGCUGCUGGGCCUGAUGCACUGGGCCAGCGCCGCCCUG |
| | 2_concat_ | UGCACCUUCCUGCUGAACAAGGAGAUGUACCUGAAGCUGCGCAGC |
| | 2.5 | GACGUGCUGCUGCCCAAGAUCUUCGUGGACGGCGUGCCCUUCGUG |
| | DC | GUGAGCACCGGCUACCACUUCCGCGAGCUGGGCAGCGCCGGCUUC |
| | L_opt2 | CCCUUCAACAAGUGGGGCAAGGCCCGCCUGUACUACGACAGCAUG |
| | | AGGUACGACAUCAAGGACCUCCCCAAGGAGAUCACCGUGGCCACC |
| | | AGCCGCACCCUGAGCUAGUACAAGGGCACCCUGAUGAUCGAGCGC |
| | | UUCGUGAGCCUGGCCAUCGACGCCUACCCCCUGACCAAGCACGAG |
| | | CUGUUCGAGAACAAGACCACCCUGCCCGUGAACGUGGCCUUCGAG |
| | | CUGUGGGCCAAGCGCGCCAGCAAGAUCCUGGGCCUGCCCACCCAG |
| | | ACCGUGGACAGCAGCCAGGGCAGCGAGUACGACAAGCUGUUCGCC |
| | | GCCGAGACCCUGAAGGCCACCGAGGAGACCUUCAAGCUGAGCUAC |
| | | GGCAUCAUCAGCCGCCAGCGCCUGACCAAGUACACCAUGGCCGAC |
| | | CUGGUGUACGCCCUGCGCCACUUCAUGUACAGCUUCGUGAGCGAG |
| | | GAGACCGGCACCCUGAUCGUGAACAGCGUGCUGCUGUUCCUGAGC |
| | | AGCGACUACACCAUCGUGCUGCCCGUGAUCGGCGCCAUCGUGGUG |
| | | GGCCUGUGCCUGAUGGGCAUGGGCGUGUACAAGAUCCGCCUGCGC |
| | | UGCCAGAGCAGCGGCUACCAGCGCAUCUAA |
| 130 | SARS- | AUGGCCGCCCCAGGCAGCGCCAGGAGACCACUGCUCCUGCUGCUG |
| | CoV- | CUGCUGCUGCUCCUGGGGCUGAUGCACUGCGCCUCUGCCGCUCUG |
| | 2_concat_ | UGCACCUUCCUGCUGAACAAGGAGAUGUAUCUGAAGCUGAGGUCC |
| | 2.5 | GACGUGCUGCUGCCAAAAAUUUUCGUGGACGGCGUGCCCUUUGUG |
| | DC | GUGUCCACCGGCUACCACUUCAGGGAGGUGGGGAGCGCCGGCUUC |
| | L_opt3 | CCCUUUAACAAGUGGGGAAAAGCCAGACUGUACUACGAUAGCAUG |
| | | AGCUACGACAUCAAAGAUCUGCCUAAGGAGAUCACAGUGGCCACA |
| | | UCCAGAACCCUGAGUUACUACAAGGGAACCCUCAUGAUCGAGAGG |
| | | UUCGUGAGCCUGGCCAUCGACGCCUACCCUCUGACCAAGCACGAG |
| | | CUGUUUGAAAACAAAACCACACUGCCCGUCAACGUGGCUUUCGAG |
| | | CUGUGGGGCAAGAGGGCCUCUAAAAUCCUGGGCCUGCGUACCCAG |
| | | ACCGUGGACUCCUCCCAGGGCUCUGAAUACGAUAAACUGUUCGCC |
| | | GCCGAGACCCUGAAAGCCACCGAGGAGACCUUCAAACUGUCCUAC |
| | | GGAAUCAUCAGCCGCCAGAGGCUGACCAAGUACACCAUGGCUGAC |
| | | CUCGUGUACGCCCUGCGGCAUUUUAUGUACAGCUUCGUGAGUGAG |
| | | GAGACCGGUACACUGAUCGUGAACAGCGUGCUGCUGUUUUUGUCU |
| | | AGCGACUACACCAUCGUGCUGCCCGUCAUCGGCGCGAUCGUGGUG |
| | | GGCCUGUGCGUGAUGGGCAUGGGUGUGUAUAAGAUCCGGCUGAGG |
| | | UGUCAGAGCUCUGGCUACCAGAGAAUCUGA |

TABLE 5
Codon optimized RNA sequences of the SARS-CoV-2 spike glycoprotein construct. (_opt1;
_opt2 refer to two differently codon-optimized sequences of the same construct).

| SEQ | | |
|---|---|---|
| ID | Name | Sequence |
| 131 | SARS- | AUGUUCGUGUUCCUGGUGCUGCUGCCUCUGGUGUCCAGCCAGUGU |
| | CoV- | GUGAACCUCCGGACCAGAACACAGCUGCCUCCAGCCUACACCAAC |
| | 2_S_PP_ | AGCUUUACCAGAGGCGUGUACUACCCCGACAAGGUGUUCAGAUCC |
| | delta_opt | AGCGUGCUGCACUCUACCCAGGACCUGUUCCUGCCUUUCUUCAGC |
| | 1 | AACGUGACCUGGUUCCACGCCAUCCACGUGUCCGGCACCAAUGGC |
| | | ACCAAGAGAUUCGACAACCCCGUGCUGCCCUUCAACGACGGGGUG |
| | | UACUUUGCCAGCACCGAGAAGUCCAACAUCAUCAGAGGCUGGAUC |
| | | UUCGGCACCACACUGGACAGCAAGACCCAGAGCCUGCUGAUCGUG |
| | | AACAACGCCACCAACGUGGUCAUCAAAGUGUGCGAGUUCCAGUUC |
| | | UGCAACGACCCCUUCCUGGACGUGUACUAUCACAAGAACAACAAG |
| | | AGCUGGAUGGAAAGCGGCGUGUACAGCAGCGCCAACAACUGCACC |
| | | UUCGAGUACGUGUCCCAGCCUUUCCUGAUGGACCUGGAAGGCAAG |
| | | CAGGGCAACUUCAAGAACCUCCGCGAGUUCGUGUUUAAGAACAUC |
| | | GACGGCUACUUCAAGAUCUACAGCAAGCACACCCCUAUCAACCUC |
| | | GUGGGGGAUCUGCCUCAGGGCUUCUCUGCUCUGGAACCCCUGGUG |
| | | GAUCUGCCCAUCGGCAUCAACAUCACCCGGUUUCAGACACUGCUG |
| | | GCCCUGCACAGAAGCUACCUGACACCUGGCGAUAGCAGCAGCGGA |
| | | UGGAGAGCUGGUGCCGCCGCUUACUAUGUGGGCUACCUCCAGCCU |
| | | AGAACCUUCCUGCUGAAGUACAACGAGAACGGCACCAUCACCGAC |
| | | GCCGUGGAUUGUGCUCUGGAUCCUCUGAGCGAGACAAAGUGCACC |
| | | CUGAAGUCCUUCACCGUGGAAAAGGGCAUCUACCAGACCAGCAAC |
| | | UUCGGGGUGCAGCCCACCGAAUCCAUCGUGCGGUUCCCCAAUAUC |
| | | ACCAAUCUGUGCCCCUUCGGCGAGGUGUUCAAUGCCACCAGAUUC |
| | | GCCUCUGUGUACGCCUGGAACCGGAAGCGGAUCAGCAAUUGCGUG |
| | | GCCGACUACUCCGUGCUGUACAACUCCGCCAGCUUCAGCACCUUC |
| | | AAGUGCUACGGCGUGUCCCCUACCAAGCUGAACGACCUGUGCUUC |
| | | ACAAACGUGUACGCCGACAGCUUCGUGAUCCGGGGAGAUGAAGUG |
| | | CGGCAGAUUGCCCCUGGACAGACAGGCAAGAUCGCCGACUACAAC |
| | | UACAAGCUGCCCGACGACUUCACCGGCUGUGUGAUUGCCUGGAAC |
| | | AGCAACAACCUGGACUCCAAAGUCGGCGGCAACUACAAUUACCGG |
| | | UACAGGCUGUUCCGGAAGUCCAAUCUGAAGCCCUUCGAGOGGGAC |
| | | AUCUCCACCGAGAUCUAUCAGGCCGGCAGGAAGCCUUGCAACGGC |
| | | GUGGAAGGCUUCAACUGCUACUUCCCACUGCAGUCCUACGGCUUU |
| | | CAGCCCACAAAUGGCGUGGGCUAUCAGCCCUAGAGAGUGGUGGUG |
| | | CUGAGCUUCGAACUGCUGCAUGCCCCUGCCACAGUGUGCGGCCCU |
| | | AAGAAAAGCACCAAUCUCGUGAAGAACAAAUGCGUGAACUUCAAC |
| | | UUGAACGGCCUGACCGGCACCGGCGUGCUGACAGAGAGCAACAAG |
| | | AAGUUCCUGCCAUUCCAGCAGUUUGGCCGGGAUAUCGCCGAUACC |
| | | ACAGACGCCGUUAGAGAUCCCCAGACACUGGAAAUCCUGGACAUC |
| | | ACCCCUUGCAGCUUCGGCGGAGUGUCUGUGAUCACCCCUGGCACC |
| | | AACACCAGCAAUCAGGUGGCAGUGCUGUACCAGGGCGUGAACUGU |
| | | ACAGAGGUGCCAGUGGCCAUUCACGCCGAUCAGCUGACCCCUACU |
| | | UGGCGGGUGUACUCCACAGGCAGCAAUGUGUUUCAGACCAGAGCC |
| | | GGCUGUCUGAUCGGAGCCGAGCACGUGAACAAUAGCUACGAGUGC |
| | | GACAUCCCCAUCGGCGCUGGCAUCUGUGCCAGCUACCAGACACAG |
| | | ACCAACAGCAGGCGGAGAGCCAGAUCUGUGGCCAGCCAGAGCAUC |
| | | AUUGCCUACACAAUGUCUCUGGGCGCCGAGAAGAGCGUGGCCUAC |
| | | UCCAACAACUCUAUCGCUAUCCCCACCAACUUCACCAUCAGCGUG |
| | | ACCACAGAGAUCCUGCCUGUGUCCAUGACCAAGACCAGCGUGGAC |
| | | UGCACCAUGUACAUCUGCGGCGAUUCCACCGAGUGCUCCAACCUC |
| | | CUGCUGCAGUACGGCAGCUUCUGCACCCAGCUGAAUAGAGCCCUG |
| | | ACAGGGAUCGCCGUGGAACAGGAGAAGAACACCCAAGAGGUGUUC |
| | | GCCCAAGUGAAGCAGAUCUACAAGACCCCUCCUAUCAAGGACUUC |
| | | GGCGGCUUCAAUUUCAGCCAGAUUCUGCCCGAUCCUAGCAAGCCC |
| | | AGCAAGCGGAGCUUCAUCGAGGACCUCCUGUUCAACAAAGUGACA |
| | | CUGGCCGACGCCGGCUUCAUCAAGCAGUAUGGCGAUUGUCUGGGC |
| | | GACAUUGCCGCCAGGGAUCUGAUUUGCGCCCAGAAGUUUAACGGA |
| | | CUGACAGUGCUGCCUCCUCUGCUGACCGAUGAGAUGAUCGCCCAG |
| | | UACACAUCUGCCCUGCUGGCCGGCACAAUCACAAGCGGCUGGACA |
| | | UUUGGAGCUGGCGCCGCUCUGCAGAUCCCCUUUGCUAUGCAGAUG |
| | | GCCUACCGGUUCAACGGCAUCGGAGUGACCCAGAAUGUGCUGUAC |
| | | GAGAACCAGAAGCUGAUCGCCAACCAGUUCAACAGCGCCAUCGGG |
| | | AAGAUCCAGGACAGCCUGAGCAGCACAGCAAGCGCCCUGGGAAAG |
| | | CUGCAGAACGUCGUGAACCAGAACGCCCAGGCACUGAACACCCUG |
| | | GUCAAGCAGCUGUCCUCCAACUUCGGCGCCAUCAGCUCUGUGCUG |
| | | AACGAUAUCCUGAGCAGACUGGACCCUCCUGAGGCCGAGGUGCAG |
| | | AUCGAGAGACUGAUCACAGGCAGACUGCAGAGCCUCGAGACAUAC |
| | | GUGACCCAGCAGCUGAUCAGAGCCGCCGAGAUUAGAGCCUCUGCC |
| | | AAUCUGGCCGCCACCAAGAUGUCUGAGUGUGUGCUGGGCCAGAGC |
| | | AAGAGAGUGGACUUUUGCGGCAAGGGCUACCACCUGAUGAGCUUC |
| | | CCUCAGUCUGCACCACACGGCGUGGUGUUUCUGCACGUGACCUAC |
| | | GUGCCCGCUCAAGAGAAGAAUUUCACCACCGCUCCAGCCAUCUGC |
| | | CACGACGGCAAAGCCCACUUUCCUAGAGAAGGCGUGUUCGUGUCC |
| | | AACGGCACCCAUUGGUUCGUGACACAGCGGAACUUCUACGAGCCC |
| | | CAGAUCAUCACCACCGACAACACCUUCGUGUCUGGCAACUGCGAC |
| | | GUCGUGAUCGGCAUUGUGAACAAUACCGUGUACGACCCUCUGCAG |
| | | CCCGAGGUGGACAGCUUCAAAGAGGAACUGGACAAGUACUUUAAG |
| | | AACCACACAAGCCCCGACGUGGACCUGGGGGAUAUCAGCGGAAUC |
| | | AAUGCCAGCGUGGUCAACAUCCAGAAAGAGAUCGACCGGCUGAAC |
| | | GAGGUGGCCAAGAAUCUGAACGAGAGCCUGAUCGACCUCCAAGAA |
| | | CUGGGGAAGUACGAGCAGUACAUCAAGUGGCCUUGGUACAUCUGG |
| | | CUGGGCUUUAUCGCCGGACUGAUUGCCAUCGUGAUGGUCACAAUC |
| | | AUGCUGUGUUGCAUGACCAGCUGCUGUAGCUGCCUGAAGGGCUGU |
| | | UGUAGCUGUGGCAGCUGCUGCAAGUUCGACGAGGACGAUUCUGAG |
| | | CCCGUGCUGAAAGGGGUGAAGCUGCACUACACCUGA |
| 132 | SARS- | AUGUUCGUGUUCCUGGUGCUGCUGCCUCUGGUGUCCAGCCAGUGU |
| | CoV- | GUGAACCUCCGGACCAGAACACAGCUGCCUCCAGCCUACACCAAC |
| | 2_S_stab_ | AGCUUUACCAGAGGCGUGUACUACCCCGACAAGGUGUUCAGAUCC |
| | delta_op | AGCGUGCUGCACUCUACCCAGGACCUGUUCCUGCCUUUCUUCAGC |
| | t1 | AACGUGACCUGGUUCCACGCCAUCCACGUGUCCGGCACCAAUGGC |
| | | ACCAAGAGAUUCGACAACCCCGUGCUGCCCUUCAACGACGGGGUG |
| | | UACUUUGCCAGCACCGAGAAGUCCAAGAUCAUCAGAGGCUGGAUC |
| | | UUCGGCACCACACUGGACAGCAAGACCCAGAGCCUGCUGAUCGUG |
| | | AACAACGCCACCAACGUGGUCAUCAAAGUGUGCGAGUUCCAGUUC |
| | | UGCAACGACCCCUUCCUGGACGUGUACUAUCACAAGAACAACAAG |
| | | AGCUGGAUGGAAAGCGGCGUGUACAGCAGOGCCAACAACUGCACC |
| | | UUCGAGUACGUGUCCCAGCCUUUCCUGAUGGACCUGGAAGGCAAG |
| | | CAGGGCAACUUCAAGAACCUCCGCGAGUUCGUGUUUAAGAACAUC |
| | | GACGGCUACUUCAAGAUCUACAGCAAGCACACCCCUAUCAACCUC |
| | | GUGCGGGAUCUGCCUCAGGGCUUCUCUGCUCUGGAACCCCUGGUG |
| | | GAUCUGCCCAUCGGCAUCAACAUCACCCGGUUUGAGACACUGCUG |
| | | GCCCUGCACAGAAGCUACCUGACACCUGGCGAUAGCAGCAGCGGA |
| | | UGGACAGCUGGUGCCGCCGCUUACUAUGUGGGCUACCUCCAGCCU |
| | | AGAACCUUCCUGCUGAAGUACAACGAGAACGGCACCAUCACCGAC |
| | | GCCGUGGAUUGUGCUCUGGAUCCUCUGAGCGAGACAAAGUGCACC |
| | | CUGAAGUCGUUCACCGUGGAAAAGGGCAUGUACCAGACCAGCAAC |
| | | UUCCGGGUGCAGCCCACCGAAUCCAUCGUGCGGUUCCCCAAUAUC |
| | | ACCAAUCUGUGCCCCUUCGGCGAGGUGUUCAAUGCCACCAGAUUC |
| | | GCCUCUGUGUACGCCUGGAACCGGAAGCGGAUCAGCAAUUGCGUG |
| | | GCCGAGUACUCCGUGCUGUACAACUCCGCCAGCUUCAGCACCUUC |
| | | AAGUGCUACGGCGUGUCCCCUACCAAGCUGAACGACCUGUGCUUC |
| | | ACAAACGUGUACGCCGACAGCUUCGUGAUCCGGGGAGAUGAAGUG |
| | | CGGCAGAUUGCCCCUGGACAGACAGGCAAGAUCGCCGACUACAAG |
| | | UACAAGCUGCCCGACGACUUCACCGGCUGUGUGAUUGCCUGGAAC |
| | | AGCAACAACCUGGACUCCAAAGUCGGCGGCAACUACAAUUACCGG |
| | | UACAGGCUGUUCCGGAAGUCCAAUCUGAAGCCCUUCGAGCGGGAC |
| | | AUCUCCACCGAGAUCUAUCAGGCCGGCAGGAAGCCUUGCAACGGC |
| | | GUGGAAGGCUUCAACUGCUACUUCCCAGUGCAGUCCUACGGCUUU |
| | | CAGCCCACAAAUGGCGUGGGCUAUCAGCCCUACAGAGUGGUGGUG |
| | | CUGAGCUUCGAACUGCUGCAUGCCCCUGCCACAGUGUGCGGCCCU |
| | | AAGAAAAGCACCAAUCUCGUGAAGAACAAAUGCGUGAAGUUCAAC |
| | | UUGAACGGCCUGAGCGGCACCGGCGUGCUGACAGAGAGCAACAAG |
| | | AAGUUCCUGCCAUUCCAGCAGUUUGGCCGGGAUAUCGCCGAUACC |
| | | ACAGACGCCGUUAGAGAUCCCCAGACACUGGAAAUCCUGGACAUC |
| | | ACCCCUUGCAGCUUCGGCGGAGUGUCUGUGAUCACCCCUGGCACC |
| | | AACACCAGCAAUCAGGUGGCAGUGGUGUACCAGGGCGUGAACUGU |
| | | ACAGAGGUGCCAGUGGCCAUUCACGCCGAUCAGCUGACCCCUACU |
| | | UGGCGGGUGUACUCCACAGGCAGCAAUGUGUUUCAGACCAGAGCC |
| | | GGCUGUCUGAUCGGAGCCGAGCACGUGAACAAUAGCUACGAGUGC |
| | | GACAUCCCCAUCGGCGCUGGCAUCUGUGCCAGGUACCAGACACAG |
| | | ACCAACAGCAGAGGCUCUGCCAGCUCUGUGGCCAGCCAGAGCAUC |
| | | AUUGCCUACACAAUGUCUCUGGGCGCCGAGAACAGCGUGGCCUAC |
| | | UCCAACAACUCUAUCGCUAUCCCCACCAACUUCACCAUCAGCGUG |
| | | ACCACAGAGAUCCUGCCUGUGUCCAUGACCAAGACCAGCGUGGAC |
| | | UGCACCAUGUACAUCUGCGGCGAUUCCACCGAGUGCUCCAACCUC |
| | | CUGCUGCAGUACGGCAGCUUCUGCACCCAGCUGAAUAGAGCCCUG |
| | | ACAGGGAUCGCCGUGGAACAGGAGAAGAACACCCAAGAGGUGUUC |
| | | GCCCAAGUGAAGCAGAUCUACAAGACCCCUCCUAUCAAGGACUUC |
| | | GGCGGCUUCAAUUUCAGCCAGAUUCUGCCCGAUCCUAGCAAGCCC |
| | | AGCAAGCGGAGCCGUAUCGAGGACCUCCUGUUCAACAAAGUGACA |
| | | CUGGCCGACGCCGGCUUCAUCAAGCAGUAUGGCGAUUGUCUGGGC |
| | | GACAUUGCCGCCAGGGAUCUGAUUUGCGCCCAGAAGUUUAACGGA |
| | | CUGACAGUGCUGCCUCCUCUGCUGACCGAUGAGAUGAUCGCCCAG |
| | | UACACAUCUGCCCUGCUGGCCGGCACAAUCACAAGGGGCUGGACA |
| | | UUUGGAGCCGGGCCUGCUCUGCAGAUGCCAUUUCCAAUGCAGAUG |
| | | GCGUACCGGUUCAACGGCAUCGGAGUGACCCAGAAUGUGCUGUAC |
| | | GAGAACCAGAAGCUGAUCGCCAACCAGUUCAACAGCGCCAUCGGC |
| | | AAGAUCCAGGACAGCCUGAGGAGCACACCUAGCGCUCUGGGCAAG |
| | | CUGCAGAACGUCGUGAACGAGAAUGCCCAGGCACUGAACACCCUG |
| | | GUCAAGCAGCUGUCCUCCAACUUCGGCGCCAUCUCUAGCGUGCUG |
| | | AACGAUAUCCUGAGCAGACUGGACCCUCCUGAGGCCGAGGUGCAG |
| | | AUCGAGAGACUGAUCACAGGCAGACUCCAGUCUCUGGAGACCUAC |
| | | GUGACCCAGCAGCUGAUGAGAGCCGCCGAGAUUAGAGCCAGCGCU |
| | | AAUCUGGCCGCCACCAAGAUGUCUGAGUGUGUGCUGGGCCAGAGC |
| | | AAGAGAGUGGACUUUUGCGGCAAGGGCUACCACCUGAUGAGCUUC |
| | | CCUCAGUCUGCACCACACGGCGUGGUGUUUCUGCACGUGACAUAC |
| | | GUGCCCGCUCAAGAGAAGAAUUUCACCACCGCUCCAGCCAUCUGC |
| | | CACGACGGCAAAGCCCACUUUCCUAGAGAAGGCGUGUUCGUGUCC |
| | | AACGGCACCCAUUGGUUCGUGACACAGCGGAACUUCUACGAGCCC |
| | | CAGAUCAUCACCACCGACAACACCUUCGUGUCUGGCAACUGUGAC |
| | | GUCGUGAUCGGCAUUGUGAACAAUACCGUGUACGACCCUCUGCAG |
| | | CCCGAGCUGGACAGCUUCAAAGAGGAACUGGACAAGUACUUUAAG |
| | | AACCACACAAGCCCCGACGUGGACCUGGGCGAUAUCAGCGGAAUC |
| | | AAUGCCAGCGUGGUCAACAUCCAGAAAGAGAUCGACCGGCUGAAC |
| | | GAGGUGGCCAAGAAUCUGAACGAGAGCCUGAUCGACCUCCAAGAA |
| | | CUGGGGAAGUACGAGCAGUACAUCAAGUGGCCUUGGUACAUCUGG |
| | | CUGGGCUUUAUCGCCGGACUGAUUGCCAUCGUGAUGGUCACAAUC |
| | | AUGCUGUGCUGCAUGACCAGCUGCUGUAGCUGCCUGAAGGGCUGU |
| | | UGUAGGUGUGGCAGCUGCUGCAAGUUCGAGGAGGACGAUUCUGAG |
| | | CCCGUGCUGAAAGGCGUGAAGCUGCACUACACCUGA |
| 133 | SARS- | AUGUUCGUGUUCCUGGUGCUGGUGCCCCUGGUGAGGAGCCAGUGC |
| | CoV- | GUGAACCUCCGCACCCGCACCCAGCUGCCCCCCGCCUACACCAAC |
| | 2_S_PP_ | AGCUUCACCCGCGGCGUGUACUACCCCGACAAGGUGUUCCGCAGC |
| | delta_opt | AGCGUGCUGCACAGCACCCAGGACCUGUUCCUGCCCUUCUUCAGC |
| | 2 | AACGUGACCUGGUUCCACGCCAUCCACGUGAGCGGCACCAACGGC |
| | | ACCAAGCGCUUCGACAACCCCGUGCUGCCCUUCAACGACGGCGUG |
| | | UACUUCGCCAGCACCGAGAAGAGGAAGAUGAUGGGGGGCUGGAUC |
| | | UUCGGCACCACCCUGGACAGCAAGACCCAGAGCCUGCUGAUCGUG |
| | | AACAACGCCACCAACGUGGUGAUCAAGGUGUGCGAGUUCCAGUUC |
| | | UGCAACGACCCCUUCCUGGACGUGUACUACCACAAGAACAACAAG |
| | | AGCUGGAUGGAGAGGGGGGUGUACAGCAGCGCCAACAACUGCACC |
| | | UUCGAGUACGUGAGCCAGCCCUUCCUGAUGGACCUGGAGGGCAAG |
| | | CAGGGCAACUUCAAGAACCUCCGCGAGUUCGUGUUCAAGAACAUC |
| | | GACGGCUACUUCAAGAUCUACAGCAAGCACACCCCCAUCAACCUG |
| | | GUGGGGGACCUCCCCCAGGGCUUCAGCGCCCUGGAGCCCCUGGUG |
| | | GACCUCCCCAUCGGCAUCAACAUCACCCGCUUCCAGACCCUGCUG |
| | | GCCCUGCACCGCAGCUACCUGACCCCCGGCGACAGCAGCAGCGGC |
| | | UGGACCGCCGGCGCCGCCGCCUACUACGUGGGCUACCUCCAGCCC |
| | | CGCACCUUCCUGCUGAAGUACAACGAGAACGGCACCAUCACCGAG |
| | | GCCGUGGACUGCGCCCUGGACCCCCUGAGCGAGACCAAGUGGACC |
| | | CUGAAGAGCUUCACCGUGGAGAAGGGCAUCUACCAGACCAGCAAC |
| | | UUCCGCGUGCAGCCCACCGAGAGCAUCGUGCGCUUCCCCAACAUC |
| | | ACCAACCUGUGCCCCUUCGGCGAGGUGUUCAACGCCACCCGCUUC |
| | | GCCAGCGUGUACGCCUGGAACCGGAAGCGCAUCAGCAACUGCGUG |
| | | GCCGACUACAGCGUGCUGUACAACAGCGCCAGCUUCAGCACCUUC |
| | | AAGUGCUACGGCGUGAGCCCCACCAAGGUGAACGACCUGUGCUUC |
| | | ACCAAGGUGUACGCCGAGAGCUUCGUGAUCCGCGGCGACGAGGUG |
| | | CGCCAGAUCGCCCCCGGCCAGACCGGCAAGAUCGCCGACUACAAC |
| | | UACAAGCUGCCCGACGACUUCACCGGCUGCGUGAUCGCCUGGAAC |
| | | AGCAACAACCUGGACAGCAAGGUGGGGGGCAAGUACAAGUACCGC |
| | | UACCGCCUGUUCCGGAAGAGCAACCUGAAGCCCUUCGAGCGCGAC |
| | | AUCAGCACCGAGAUCUACCAGGCCGGCAGCAAGCCCUGCAACGGC |
| | | GUGGAGGGCUUCAACUGCUACUUCCCCCUGCAGAGCUACGGCUUC |
| | | CAGCCCACCAACGGCGUGGGCUACCAGCCCUACCGCGUGGUGGUG |
| | | CUGAGCUUCGAGCUGCUGCACGCCCCCGCCACCGUGUGCGGCCCC |
| | | AAGAAGAGCACCAACCUGGUGAAGAACAAGUGCGUGAACUUCAAC |
| | | UUCAACGGCCUGACCGGCACCGGCGUGCUGACCGAGAGCAACAAG |
| | | AAGUUCCUGCCCUUCCAGCAGUUCGGCCGCGACAUCGCCGACACC |
| | | ACCGACGCCGUGCGCGACCCCCAGACCCUGGAGAUCCUGGACAUC |
| | | ACCCCCUGCAGCUUCGGCGGCGUGAGCGUGAUCACCCCCGGCACC |
| | | AACACCAGCAACCAGGUGGCCGUGCUGUACCAGGGCGUGAACUGC |
| | | ACCGAGGUGCCCGUGGCCAUCCACGCCGACCAGCUGACCCCCACC |
| | | UGGCGCGUGUACAGCACCGGCAGCAACGUGUUCCAGACCCGCGCC |
| | | GGCUGCCUGAUCGGCGCCGAGCACGUGAACAACAGCUACGAGUGC |
| | | GAGAUCCCCAUCGGCGCCGGCAUCUGCGCCAGCUACCAGACCCAG |
| | | ACCAACAGCCGCCGCCGCGCCCGCAGCGUGGCCAGCCAGAGCAUC |
| | | AUCGCCUACACCAUGAGCCUGGGCGCCGAGAACAGCGUGGCCUAC |
| | | AGCAACAACAGCAUCGCCAUCCCCACCAACUUCACCAUCAGCGUG |
| | | ACCACCGAGAUCCUGCCCGUGAGCAUGACCAAGACGAGCGUGGAC |
| | | UGCACCAUGUACAUCUGCGGCGACAGCACCGAGUGCAGCAACCUC |
| | | CUGCUGCAGUACGGCAGCUUCUGCACCCAGCUGAACCGCGCCCUG |
| | | ACCGGCAUCGCCGUGGAGCAGGACAAGAACACCCAGGAGGUGUUC |
| | | GCCCAGGUGAAGCAGAUGUACAAGACCCCCCCCAUGAAGGACUUC |
| | | GGCGGCUUCAACUUCAGCCAGAUCCUGCCCGACCCCAGCAAGOCC |
| | | AGCAAGCGCAGCUUCAUCGAGGACCUCCUGUUCAACAAGGUGACC |
| | | CUGGCCGACGCCGGCUUCAUCAAGCAGUACGGCGACUGCCUGGGC |
| | | GACAUCGCCGCCCGCGACCUGAUCUGCGCCCAGAAGUUCAACGGC |
| | | CUGACCGUGCUGCCCCCCCUGCUGACCGACGAGAUGAUCGCCCAG |
| | | UACACCAGCGCCCUGCUGGCCGGCACCAUCACCAGCGGCUGGACC |
| | | UUCGGCGCCGGCGCCGCCCUGCAGAUCCCCUUCGCCAUGCAGAUG |
| | | GCCUACCGCUUCAACGGCAUCGGCGUGACCCAGAACGUGCUGUAC |
| | | GAGAACCAGAAGCUGAUCGCCAACCAGUUCAACAGCGCCAUCGGC |
| | | AAGAUCCAGGACAGCCUGAGCAGCACCGCCAGCGCCCUGGGCAAG |
| | | CUGCAGAACGUGGUGAACCAGAACGCCCAGGCCCUGAACACCCUG |
| | | GUGAAGGAGCUGAGGAGCAACUUCGGCGCCAUGAGCAGCGUGGUG |
| | | AACGAGAUCGUGAGCCGCCUGGACCCCCCCGAGGCCGAGGUGCAG |
| | | AUCGACCGCCUGAUCACCGGCCGCCUGCAGAGCCUGCAGACCUAC |
| | | GUGACCCAGCAGCUGAUCCGCGCCGCCGAGAUCCGCGCCAGCGCC |
| | | AACCUGGCCGCCACCAAGAUGAGCGAGUGCGUGCUGGGCCAGAGC |
| | | AAGCGCGUGGACUUCUGCGGCAAGGGCUACCACCUGAUGAGCUUC |
| | | CCCCAGAGCGCCCCCCACGGCGUGGUGUUCCUGCACGUGACCUAC |
| | | GUGCCCGCCCAGGAGAAGAACUUCACCACCGCCCCCGCCAUCUGC |
| | | CACGACGGCAAGGCCCACUUCCCCCGCGAGGGCGUGUUCGUGAGC |
| | | AACGGCACCCACUGGUUCGUGACCCAGCGCAACUUCUACGAGCCC |
| | | CAGAUCAUCACCACCGACAACACCUUCGUGAGCGGCAACUGCGAC |
| | | GUGGUGAUCGGCAUCGUGAACAACACCGUGUACGACCCCCUGCAG |
| | | CCCGAGCUGGACAGCUUCAAGGAGGAGCUGGACAAGUACUUCAAG |
| | | AACCACACCAGCCCCGACGUGGACCUGGGCGACAUCAGCGGCAUC |
| | | AACGCCAGCGUGGUGAACAUCCAGAAGGAGAUCGACCGCCUGAAC |
| | | GAGGUGGCCAAGAACCUGAACGAGAGCCUGAUCGACCUCCAGGAG |
| | | CUGGGCAAGUACGAGCAGUACAUCAAGUGGGCCUGGUACAUCUGG |
| | | CUGGGCUUCAUCGCCGGCCUGAUCGCCAUCGUGAUGGUGACCAUC |
| | | AUGCUGUGCUGCAUGACCAGCUGCUGCAGCUGCCUGAAGGGCUGC |
| | | UGCAGCUGCGGCAGCUGCUGCAAGUUCGACGAGGACGACAGCGAG |
| | | CCCGUGCUGAAGGGCGUGAAGCUGCACUACACCUAA |
| 134 | SARS- | AUGUUCGUGUUCCUGGUGCUGCUGCCCCUGGUGAGCAGCCAGUGC |
| | CoV- | GUGAACCUCCGCACCCGCACCCAGCUGCCCCCCGCCUACACCAAC |
| | 2_S_stab_ | AGCUUCACCCGCGGCGUGUAGUACCCCGACAAGGUGUUCCGCAGC |
| | delta_ | AGCGUGCUGCACAGCACCCAGGACCUGUUCCUGCCCUUCUUCAGC |
| | opt2 | AACGUGACCUGGUUCCACGCCAUCCACGUGAGCGGCACCAACGGC |
| | | ACCAAGCGCUUCGACAACCCCGUGCUGCCCUUCAACGACGGCGUG |
| | | UACUUCGCCAGCACCGAGAAGAGCAACAUCAUCCGGGGCUGGAUC |
| | | UUCGGCACCACCCUGGACAGCAAGACCCAGAGCCUGCUGAUCGUG |
| | | AACAACGCCACCAACGUGGUGAUCAAGGUGUGCGAGUUCCAGUUC |
| | | UGCAACGACCCCUUCCUGGACGUGUACUACCACAAGAACAACAAG |
| | | AGCUGGAUGGAGAGCGGCGUGUACAGCAGCGCCAACAACUGCACC |
| | | UUCGAGUACGUGAGCCAGCCCUUCCUGAUGGACCUGGAGGGGAAG |
| | | CAGGGCAACUUCAAGAACCUCCGCGAGUUCGUGUUCAAGAACAUC |
| | | GACGGCUACUUCAAGAUCUACAGCAAGCACACCCCCAUCAACCUG |
| | | GUGCGCGACCUCCCCCAGGGCUUCAGCGCCCUGGAGCCCCUGGUG |
| | | GACCUCCCCAUCGGCAUCAACAUCACCCGCUUCCAGACCCUGCUG |
| | | GCCCUGCACCGCAGCUACCUGACCCCCGGCGACAGCAGCAGCGGC |
| | | UGGACCGCCGGCGCCGCCGCCUACUACGUGGGCUACCUCCAGCCC |
| | | CGCACCUUCCUGCUGAAGUACAACGAGAACGGCACCAUCACCGAC |
| | | GCCGUGGACUGCGCCCUGGACCCCCUGAGCGAGACCAAGUGCACC |
| | | CUGAAGAGCUUCACCGUGGAGAAGGGCAUCUACCAGACCAGCAAC |
| | | UUCCGCGUGCAGCCCACCGAGAGCAUCGUGCGCUUCCCCAACAUC |
| | | ACCAACCUGUGCCCCUUCGGCGAGGUGUUCAACGCCACCCGCUUC |
| | | GCCAGCGUGUACGCCUGGAACCGCAAGCGCAUGAGGAACUGCGUG |
| | | GCCGACUACAGCGUGCUGUACAACAGCGCCAGCUUCAGCACCUUC |
| | | AAGUGCUACGGCGUGAGCCCCACCAAGCUGAACGACCUGUGCUUC |
| | | ACCAACGUGUACGCCGACAGCUUCGUGAUCCGCGGCGACGAGGUG |
| | | CGCCAGAUCGCCCCCGGCCAGACCGGCAAGAUCGCCGAGUACAAG |
| | | UACAAGCUGCCCGACGACUUCACCGGCUGCGUGAUCGCCUGGAAG |
| | | AGCAACAACCUGGACAGCAAGGUGGGGGGCAACUACAACUACCGC |
| | | UACCGCCUGUUCCGCAAGAGCAACCUGAAGCCCUUCGAGCGCGAC |
| | | AUCAGCACCGAGAUCUACCAGGCCGGCAGCAAGCCCUGCAACGGC |
| | | GUGGAGGGCUUCAACUGCUACUUCCCCCUGCAGAGCUACGGCUUC |
| | | CAGCCCACCAACGGCGUGGGCUACCAGCCCUACCGCGUGGUGGUG |
| | | CUGAGCUUCGAGCUGCUGCACGCCCCCGCCACCGUGUGCGGCCCC |
| | | AAGAAGAGCACCAACCUGGUGAAGAACAAGUGCGUGAACUUCAAC |
| | | UUCAACGGCCUGACCGGCACCGGCGUGCUGACCGAGAGCAACAAG |
| | | AAGUUCCUGCCCUUCCAGCAGUUCGGCCGCGACAUCGCCGACACC |
| | | ACCGACGCCGUGCGCGACCCCCAGACCCUGGAGAUCCUGGACAUC |
| | | ACCCCCUGCAGCUUCGGCGGCGUGAGCGUGAUCACCCCCGGCACC |
| | | AACACCAGCAACCAGGUGGCCGUGCUGUACCAGGGCGUGAACUGG |
| | | ACCGAGGUGCCCGUGGCCAUCCACGCCGACCAGCUGACCCCCACC |
| | | UGGCGCGUGUACAGCACCGGCAGCAACGUGUUCCAGACCCGCGCC |
| | | GGCUGCCUGAUCGGCGCCGAGCACGUGAACAACAGCUACGAGUGC |
| | | GAGAUCCCCAUCGGCGCCGGCAUCUGCGCCAGCUACCAGACCCAG |
| | | ACCAACAGCCGCGGCAGCGCCAGCAGCGUGGCCAGCCAGAGCAUC |
| | | AUCGCCUACACCAUGAGCCUGGGCGCCGAGAACAGCGUGGCCUAC |
| | | AGCAAGAACAGCAUCGCCAUCCCCACCAACUUCACCAUCAGCGUG |
| | | ACCACCGAGAUCCUGCCCGUGAGCAUGACCAAGACGAGCGUGGAC |
| | | UGCACCAUGUACAUCUGCGGCGACAGCACCGAGUGCAGCAACCUC |
| | | CUGCUGCAGUACGGCAGCUUCUGCACCCAGCUGAACCGCGCCCUG |
| | | ACCGGCAUCGCCGUGGAGCAGGACAAGAACACCCAGGAGGUGUUC |
| | | GCCCAGGUGAAGCAGAUGUACAAGACCCCCCCCAUGAAGGAGUUC |
| | | GGCGGCUUCAACUUCAGCCAGAUCCUGCCCGACCCCAGCAAGCCC |
| | | AGGAAGCGCAGCCCCAUCGAGGACCUCCUGUUCAACAAGGUGACC |
| | | CUGGCCGACGCCGGCUUCAUCAAGCAGUACGGCGACUGCCUGGGC |
| | | GACAUCGCCGCCCGCGACCUGAUCUGCGCCCAGAAGUUCAACGGC |
| | | CUGACCGUGCUGCCCCCCCUGCUGACCGACGAGAUGAUGGCCCAG |
| | | UACACCAGCGCCCUGCUGGCCGGCACCAUCACCAGCGGCUGGACC |
| | | UUCGGCGCCGGCCCCGCCCUGCAGAUCCCCUUCCCCAUGCAGAUG |
| | | GCCUACCGCUUCAACGGCAUCGGCGUGACCCAGAACGUGCUGUAC |
| | | GAGAACCAGAAGCUGAUCGCCAACCAGUUCAACAGCGCCAUCGGC |
| | | AAGAUCCAGGACAGCCUGAGCAGCACCCCCAGCGCCCUGGGCAAG |
| | | CUGCAGAACGUGGUGAACCAGAACGCCCAGGCCCUGAACACCCUG |
| | | GUGAAGCAGCUGAGCAGCAACUUCGGCGCCAUCAGCAGCGUGCUG |
| | | AACGACAUCCUGAGCCGCCUGGACCCCCCCGAGGCCGAGGUGCAG |
| | | AUCGACCGCCUGAUCACCGGCCGCCUGCAGAGCCUGCAGACCUAC |
| | | GUGACCCAGCAGCUGAUCCGCGCCGCCGAGAUCCGCGCCAGCGCC |
| | | AACCUGGCCGCCACCAAGAUGAGCGAGUGCGUGCUGGGCCAGAGC |
| | | AAGCGCGUGGACUUCUGCGGCAAGGGCUACCACCUGAUGAGCUUC |
| | | CCCCAGAGCGCCCCCCACGGCGUGGUGUUCCUGCACGUGACCUAC |
| | | GUGCCCGCCCAGGAGAAGAACUUCACCACCGCCCCCGCCAUCUGC |
| | | CACGACGGCAAGGCCCACUUCCCCCGCGAGGGCGUGUUCGUGAGC |
| | | AACGGCACCGACUGGUUCGUGACCCAGCGCAACUUCUACGAGCCC |
| | | CAGAUCAUCACCACCGACAACACCUUCGUGAGCGGCAACUGCGAC |
| | | GUGGUGAUCGGCAUCGUGAACAACACCGUGUACGACCCCCUGCAG |
| | | CCCGAGCUGGACAGCUUCAAGGAGGAGCUGGACAAGUACUUCAAG |
| | | AACCACACCAGCCCCGACGUGGACCUGGGCGACAUCAGCGGCAUC |
| | | AACGCCAGCGUGGUGAACAUCCAGAAGGAGAUCGACCGCCUGAAC |
| | | GAGGUGGCCAAGAACCUGAACGAGAGCCUGAUCGACCUCCAGGAG |
| | | CUGGGCAAGUACGAGCAGUACAUCAAGUGGCCCUGGUACAUCUGG |
| | | CUGGGCUUCAUCGCCGGCCUGAUCGCCAUCGUGAUGGUGACCAUC |
| | | AUGCUGUGCUGCAUGACCAGCUGCUGCAGCUGCCUGAAGGGCUGC |
| | | UGCAGCUGGGGCAGCUGCUGGAAGUUCGACGAGGACGACAGCGAG |
| | | CCCGUGCUGAAGGGCGUGAAGCUGCACUACACCUAA |

### REFERENCES

Wu, F., Zhao, S., Yu, B. et al. A new coronavirus associated with human respiratory disease in China. Nature 579, 265-269 (2020).

1. A multi-epitope nucleic acid construct encoding peptides or functional variants and/or fragments thereof derived from a coronavirus wherein said peptides, variants and/or fragments thereof comprise amino acid sequences having at least 95% sequence identity to SEQ ID NO: 1, 2, 7, 9, 10, 12, 13, 16, 18, 19, 20, 24, 28, 31, 32, 33, 35, 37, 42, and 44.

2. The multi-epitope construct according to claim 1, wherein said construct comprises a sequence selected from the list comprising: SEQ ID NO: 116, 117, or 118; in particular 117; or sequences having at least 95% sequence identity thereto.

3. The multi-epitope construct according to any one of claims 1-2, further comprising one or more nucleic acid sequences encoding a coronaviral glycoprotein or functional variants and/or fragments thereof, in particular a SARS-COV-2 spike glycoprotein.

4. A combination comprising the multi-epitope construct according to anyone of claims 1 to 3, and a construct comprising one or more nucleic acid sequences encoding a coronaviral glycoprotein or functional variants and/or fragments thereof, in particular a SARS-COV-2 spike glycoprotein.

5. The multi-epitope construct according to anyone of claims 1 to 3, or the combination according to claim 4, wherein said encoded peptides or functional variants and/or fragments thereof are separated with at least one molecular linker selected from the list comprising: a flexible linker, a rigid linker, and/or a cleavable linker.

6. A polypeptide encoded by the multi-epitope construct according to any one of claim 1 to 3 or 5.

7. A pharmaceutical composition comprising the multi-epitope construct according to any one of claims 1 to 3 or claim 5, the combination according to claim 4, or the polypeptide according to claim 6, and at least one pharmaceutically acceptable agent.

8. The multi-epitope construct according to any one of claim 1 to 3 or 5, the combination according to claim 4, the polypeptide according to claim 6 or the pharmaceutical composition according to claim 7; which is formulated in liposomes or nanoparticles, such as lipid nanoparticles or polymeric nanoparticles; in particular lipid nanoparticles.

9. The multi-epitope construct according to any one of claim 1 to 3 or 5, the combination according to claim 4, the polypeptide according to claim 6 or the pharmaceutical composition according to claim 8; for use in human or veterinary medicine.

10. The multi-epitope construct according to any one of claim 1 to 3 or 5, the combination according to claim 4, the polypeptide according to claim 6 or the pharmaceutical composition according to claim 8; for use in vaccination, in particular intramuscular vaccination.

11. The multi-epitope construct according to any one of claim 1 to 3 or 5, the combination according to claim 4, the polypeptide according to claim 6 or the pharmaceutical composition according to claim 8; for use of inducing an immune response against a coronavirus in a subject; in particular the SARS-COV-2 virus.

12. The multi-epitope construct according to any one of claim 1 to 3 or 5, the combination according to claim 4, the polypeptide according to claim 6 or the pharmaceutical composition according to claim 8; for use in the treatment or prevention of a coronavirus infection in a subject; in particular the SARS-COV-2 virus.

13. A method of inducing an immune response against a coronavirus, comprising: administering a therapeutically effective amount of the multi-epitope construct according to any one of claim 1 to 3 or 5, the combination according to claim 4, the polypeptide according to claim 6 or the pharmaceutical composition according to claim 8 to a subject.


SEQUENCE LISTING (136 sequences)

SEQ ID NO: 1 - protein, 21 aa, synthetic construct
  REGION 1..21: note = >E_1-21 SEQ ID NO:1|descr=Homo sapiens|len=21
MYSFVSEETGTLIVNSVLLFL

SEQ ID NO: 2 - protein, 21 aa, synthetic construct
  REGION 1..21: note = >M_160-180 SEQ ID NO:2|descr=Homo sapiens|len=21
DIKDLPKEITVATSRTLSYYK

SEQ ID NO: 3 - protein, 21 aa, synthetic construct
  REGION 1..21: note = >M_37-57 SEQ ID NO:3|descr=Homo sapiens|len=21
FAYANRNRFLYIIKLIFLWLL

SEQ ID NO: 4 - protein, 21 aa, synthetic construct
  REGION 1..21: note = >M_8-28 SEQ ID NO:4|descr=Homo sapiens|len=21
ITVEELKKLLEQWNLVIGFLF

SEQ ID NO: 5 - protein, 21 aa, synthetic construct
  REGION 1..21: note = >N_105-125 SEQ ID NO:5|descr=Homo sapiens|len=21
SPRWYFYYLGTGPEAGLPYGA

SEQ ID NO: 6 - protein, 21 aa, synthetic construct
  REGION 1..21: note = >N_296-316 SEQ ID NO:6|descr=Homo sapiens|len=21
TDYKHWPQIAQFAPSASAFFG

SEQ ID NO: 7 - protein, 21 aa, synthetic construct
  REGION 1..21: note = >N_302-322 SEQ ID NO:7|descr=Homo sapiens|len=21
PQIAQFAPSASAFFGMSRIGM

SEQ ID NO: 8 - protein, 21 aa, synthetic construct
  REGION 1..21: note = >NS6(ORF6)_1-21 SEQ ID NO:8|descr=Homo sapiens|len=21
MFHLVDFQVTIAEILLIIMRT

SEQ ID NO: 9 - protein, 21 aa, synthetic construct
  REGION 1..21: note = >NSP1(ORF1a)_52-72 SEQ ID NO:9|descr=Homo sapiens|len=21
GLVEVEKGVLPQLEQPYVFIK

SEQ ID NO: 10 - protein, 21 aa, synthetic construct
  REGION 1..21: note = >NSP12(ORF1b)_114-134 SEQ ID NO:10|descr=Homo sapiens|len=21
ISRQRLTKYTMADLVYALRHF

SEQ ID NO: 11 - protein, 21 aa, synthetic construct
  REGION 1..21: note = >NSP12(ORF1b)_231-251 SEQ ID NO:11|descr=Homo sapiens|len=21
VPVVDSYYSLLMPILTLTRAL

SEQ ID NO: 12 - protein, 21 aa, synthetic construct
  REGION 1..21: note = >NSP12(ORF1b)_332-352 SEQ ID NO:12|descr=Homo sapiens|len=21
KIFVDGVPFVVSTGYHFRELG

SEQ ID NO: 13 - protein, 21 aa, synthetic construct
  REGION 1..21: note = >NSP12(ORF1b)_501-521 SEQ ID NO:13|descr=Homo sapiens|len=21
SAGFPFNKWGKARLYYDSMSY

SEQ ID NO: 14 - protein, 21 aa, synthetic construct
  REGION 1..21: note = >NSP12(ORF1b)_559-579 SEQ ID NO:14|descr=Homo sapiens|len=21
GVSICSTMTNRQFHQKLLKSI

SEQ ID NO: 15 - protein, 21 aa, synthetic construct
  REGION 1..21: note = >NSP12(ORF1b)_616-636 SEQ ID NO:15|descr=Homo sapiens|len=21
GWDYPKCDRAMPNMLRIMASL

SEQ ID NO: 16 - protein, 21 aa, synthetic construct
  REGION 1..21: note = >NSP12(ORF1b)_64-84 SEQ ID NO:16|descr=Homo sapiens|len=21
NLIDSYFVVKRHTFSNYQHEE

SEQ ID NO: 17 - protein, 21 aa, synthetic construct
  REGION 1..21: note = >NSP12(ORF1b)_680-700 SEQ ID NO:17|descr=Homo sapiens|len=21
TSSGDATTAYANSVFNICQAV

SEQ ID NO: 18 - protein, 21 aa, synthetic construct
  REGION 1..21: note = >NSP12(ORF1b)_852-872 SEQ ID NO:18|descr=Homo sapiens|len=21
GTLMIERFVSLAIDAYPLTKH

SEQ ID NO: 19 - protein, 21 aa, synthetic construct
  REGION 1..21: note = >NSP12(ORF1b)_886-906 SEQ ID NO:19|descr=Homo sapiens|len=21
QYIRKLHDELTGHMLDMYSVM

SEQ ID NO: 20 - protein, 21 aa, synthetic construct
  REGION 1..21: note = >NSP13(ORF1b)_131-151 SEQ ID NO:20|descr=Homo sapiens|len=21
KLFAAETLKATEETFKLSYGI

SEQ ID NO: 21 - protein, 21 aa, synthetic construct
  REGION 1..21: note = >NSP13(ORF1b)_228-248 SEQ ID NO:21|descr=Homo sapiens|len=21
TSHTVMPLSAPTLVPQEHYVR

SEQ ID NO: 22 - protein, 21 aa, synthetic construct
  REGION 1..21: note = >NSP13(ORF1b)_372-392 SEQ ID NO:22|descr=Homo sapiens|len=21
VFDEISMATNYDLSVVNARLR

SEQ ID NO: 23 - protein, 21 aa, synthetic construct
  REGION 1..21: note = >NSP13(ORF1b)_394-414 SEQ ID NO:23|descr=Homo sapiens|len=21
KHYVYIGDPAQLPAPRTLLTK

SEQ ID NO: 24 - protein, 21 aa, synthetic construct
  REGION 1..21: note = >NSP13(ORF1b)_522-542 SEQ ID NO:24|descr=Homo sapiens|len=21
ASKILGLPTQTVDSSQGSEYD

SEQ ID NO: 25 - protein, 21 aa, synthetic construct
  REGION 1..21: note = >NSP14(ORF1b)_175-195 SEQ ID NO:25|descr=Homo sapiens|len=21
KNLSDRVVFVLWAHGFELTSM

SEQ ID NO: 26 - protein, 21 aa, synthetic construct
  REGION 1..21: note = >NSP14(ORF1b)_228-248 SEQ ID NO:26|descr=Homo sapiens|len=21
HHSIGFDYVYNPFMIDVQQWG

SEQ ID NO: 27 - protein, 21 aa, synthetic construct
  REGION 1..21: note = >NSP14(ORF1b)_320-430 SEQ ID NO:27|descr=Homo sapiens|len=21
ALLADKFPVLHDIGNPKAIKC

SEQ ID NO: 28 - protein, 21 aa, synthetic construct
  REGION 1..21: note = >NSP14(ORF1b)_458-478 SEQ ID NO:28|descr=Homo sapiens|len=21
QVVSDIDYVPLKSATCITRCN

SEQ ID NO: 29 - protein, 21 aa, synthetic construct
  REGION 1..21: note = >NSP14(ORF1b)_500-520 SEQ ID NO:29|descr=Homo sapiens|len=21
MMISAGFSLWVYKQFDTYNLW

SEQ ID NO: 30 - protein, 21 aa, synthetic construct
  REGION 1..21: note = >NSP14(ORF1b)_504-524 SEQ ID NO:30|descr=Homo sapiens|len=21
AGFSLWVYKQFDTYNLWNTFT

SEQ ID NO: 31 - protein, 21 aa, synthetic construct
  REGION 1..21: note = >NSP15(ORF1b)_292-312 SEQ ID NO:31|descr=Homo sapiens|len=21
CSVIDLLLDDFVEIIKSQDLS

SEQ ID NO: 32 - protein, 21 aa, synthetic construct
  REGION 1..21: note = >NSP15(ORF1b)_41-61 SEQ ID NO:32|descr=Homo sapiens|len=21
ELFENKTTLPVNVAFELWAKR

SEQ ID NO: 33 - protein, 21 aa, synthetic construct
  REGION 1..21: note = >NSP16(ORF1b)_53-73 SEQ ID NO:33|descr=Homo sapiens|len=21
YLNTLTLAVPYNMRVIHFGAG

SEQ ID NO: 34 - protein, 21 aa, synthetic construct
  REGION 1..21: note = >NSP3(ORF1a)_1-21 SEQ ID NO:34|descr=Homo sapiens|len=21
APTKVTFGDDTVIEVQGYKSV

SEQ ID NO: 35 - protein, 21 aa, synthetic construct
  REGION 1..21: note = >NSP3(ORF1a)_1136-1156 SEQ ID NO:35|descr=Homo sapiens|len=21
ELKVTFFPDLNGDVVAIDYKH

SEQ ID NO: 36 - protein, 21 aa, synthetic construct
  REGION 1..21: note = >NSP3(ORF1a)_1504-1524 SEQ ID NO:36|descr=Homo sapiens|len=21
GLVAEWFLAYILFTRFFYVLG

SEQ ID NO: 37 - protein, 21 aa, synthetic construct
  REGION 1..21: note = >NSP3(ORF1a)_1549-1569 SEQ ID NO:37|descr=Homo sapiens|len=21
LIINLVQMAPISAMVRMYIFF

SEQ ID NO: 38 - protein, 21 aa, synthetic construct
  REGION 1..21: note = >NSP3(ORF1a)_209-229 SEQ ID NO:38|descr=Homo sapiens|len=21
SFSGYLKLTDNVYIKNADIVE

SEQ ID NO: 39 - protein, 21 aa, synthetic construct
  REGION 1..21: note = >NSP4(ORF1a)_331-351 SEQ ID NO:39|descr=Homo sapiens|len=21
LTPVYSFLPGVYSVIYLYLTF

SEQ ID NO: 40 - protein, 21 aa, synthetic construct
  REGION 1..21: note = >NSP4(ORF1a)_340-360 SEQ ID NO:40|descr=Homo sapiens|len=21
GVYSVIYLYLTFYLTNDVSFL

SEQ ID NO: 41 - protein, 21 aa, Homo sapiens
  REGION 1..21: note = >NSP4(ORF1a)_36-56 SEQ ID NO:41|descr=Homo sapiens|len=21
HTDFSSEIIGYKAIDGGVTRD

SEQ ID NO: 42 - protein, 21 aa, synthetic construct
  REGION 1..21: note = >NSP4(ORF1a)_417-437 SEQ ID NO:42|descr=Homo sapiens|len=21
LCTFLLNKEMYLKLRSDVLLP

SEQ ID NO: 43 - protein, 21 aa, synthetic construct
  REGION 1..21: note = >NSP5(ORF1a)_164-184 SEQ ID NO:43|descr=Homo sapiens|len=21
HMELPTGVHAGTDLEGNFYGP

SEQ ID NO: 44 - protein, 24 aa, synthetic construct
  REGION 1..24: note = >NSP6(ORF1a)_237-260 SEQ ID NO:44|descr=Homo sapiens|len=24
LTLGVYDYLVSTQEFRYMNSQGLL

SEQ ID NO: 45 - protein, 35 aa, synthetic construct
  REGION 1..35: note = >NSP8(ORF1a)_150-184 SEQ ID NO:45|descr=Homo sapiens|len=35
ASALWEIQQVVDADSKIVQLSEISMDNSPNLAWPL

SEQ ID NO: 46 - protein, 21 aa, synthetic construct
  REGION 1..21: note = >NSP8(ORF1a)_53-73 SEQ ID NO:46|descr=Homo sapiens|len=21
AAMQRKLEKMADQAMTQMYKQ

SEQ ID NO: 47 - protein, 21 aa, synthetic construct
  REGION 1..21: note = >NSP9(ORF1a)_14-34 SEQ ID NO:47|descr=Homo sapiens|len=21
CAAGTTQTACTDDNALAYYNT

SEQ ID NO: 48 - RNA, 63 nt, synthetic construct
  misc_feature 1..63: note = >E_1-21 SEQ ID NO:48|descr=Homo sapiens|len=63
  source 1..63: mol_type = other RNA
atgtactcattcgtttcggaagagacaggtacgttaatagttaatagcgtacttctttttctt

SEQ ID NO: 49 - RNA, 63 nt, synthetic construct
  misc_feature 1..63: note = >M_160-180 SEQ ID NO:49|descr=Homo sapiens|len=63
  source 1..63: mol_type = other RNA
gacatcaaggacctgcctaaagaaatcactgttgctacatcacgaacgctttcttattacaaa

SEQ ID NO: 50 - RNA, 63 nt, synthetic construct
  misc_feature 1..63: note = >M_37-57 SEQ ID NO:50|descr=Homo sapiens|len=63
  source 1..63: mol_type = other RNA
tttgcctatgccaacaggaataggtttttgtatataattaagttaattttcctctggctgtta

SEQ ID NO: 51 - RNA, 63 nt, synthetic construct
  misc_feature 1..63: note = >M_8-28 SEQ ID NO:51|descr=Homo sapiens|len=63
  source 1..63: mol_type = other RNA
attaccgttgaagagcttaaaaagctccttgaacaatggaacctagtaataggtttcctattc

SEQ ID NO: 52 - RNA, 63 nt, synthetic construct
  misc_feature 1..63: note = >N_105-125 SEQ ID NO:52|descr=Homo sapiens|len=63
  source 1..63: mol_type = other RNA
agtccaagatggtatttctactacctaggaactgggccagaagctggacttccctatggtgct

SEQ ID NO: 53 - RNA, 63 nt, synthetic construct
  misc_feature 1..63: note = >N_296-316 SEQ ID NO:53|descr=Homo sapiens|len=63
  source 1..63: mol_type = other RNA
actgattacaaacattggccgcaaattgcacaatttgcccccagcgcttcagcgttcttcgga

SEQ ID NO: 54 - RNA, 63 nt, synthetic construct
  misc_feature 1..63: note = >N_302-322 SEQ ID NO:54|descr=Homo sapiens|len=63
  source 1..63: mol_type = other RNA
ccgcaaattgcacaatttgcccccagcgcttcagcgttcttcggaatgtcgcgcattggcatg

SEQ ID NO: 55 - RNA, 63 nt, synthetic construct
  misc_feature 1..63: note = >NS6(ORF6)_1-21 SEQ ID NO:55|descr=Homo sapiens|len=63
  source 1..63: mol_type = other RNA
atgtttcatctcgttgactttcaggttactatagcagagatattactaattattatgaggact

SEQ ID NO: 56 - RNA, 63 nt, synthetic construct
  misc_feature 1..63: note = >NSP1(ORF1a)_52-72 SEQ ID NO:56|descr=Homo sapiens|len=63
  source 1..63: mol_type = other RNA
ggcttagtagaagttgaaaaaggcgttttgcctcaacttgaacagccctatgtgttcatcaaa

SEQ ID NO: 57 - RNA, 63 nt, synthetic construct
  misc_feature 1..63: note = >NSP12(ORF1b)_114-134 SEQ ID NO:57|descr=Homo sapiens|len=63
  source 1..63: mol_type = other RNA
atatcacgtcaacgtcttactaaatacacaatggcagacctcgtctatgctttaaggcatttt

SEQ ID NO: 58 - RNA, 63 nt, synthetic construct
  misc_feature 1..63: note = >NSP12(ORF1b)_231-251 SEQ ID NO:58|descr=Homo sapiens|len=63
  source 1..63: mol_type = other RNA
gttcctgttgtagattcttattattcattgttaatgcctatattaaccttgaccagggcttta

SEQ ID NO: 59 - RNA, 63 nt, synthetic construct
  misc_feature 1..63: note = >NSP12(ORF1b)_332-352 SEQ ID NO:59|descr=Homo sapiens|len=63
  source 1..63: mol_type = other RNA
aaaatatttgttgatggtgttccatttgtagtttcaactggataccacttcagagagctaggt

SEQ ID NO: 60 - RNA, 63 nt, synthetic construct
  misc_feature 1..63: note = >NSP12(ORF1b)_501-521 SEQ ID NO:60|descr=Homo sapiens|len=63
  source 1..63: mol_type = other RNA
tcagctggttttccatttaataaatggggtaaggctagactttattatgattcaatgagttat

SEQ ID NO: 61 - RNA, 63 nt, synthetic construct
  misc_feature 1..63: note = >NSP12(ORF1b)_559-579 SEQ ID NO:61|descr=Homo sapiens|len=63
  source 1..63: mol_type = other RNA
ggtgtctctatctgtagtactatgaccaatagacagtttcatcaaaaattattgaaatcaata

SEQ ID NO: 62 - RNA, 63 nt, synthetic construct
  misc_feature 1..63: note = >NSP12(ORF1b)_616-636 SEQ ID NO:62|descr=Homo sapiens|len=63
  source 1..63: mol_type = other RNA
ggttgggattatcctaaatgtgatagagccatgcctaacatgcttagaattatggcctcactt

SEQ ID NO: 63 - RNA, 63 nt, synthetic construct
  misc_feature 1..63: note = >NSP12(ORF1b)_64-84 SEQ ID NO:63|descr=Homo sapiens|len=63
  source 1..63: mol_type = other RNA
aatttaattgattcttactttgtagttaagagacacactttctctaactaccaacatgaagaa

SEQ ID NO: 64 - RNA, 63 nt, synthetic construct
  misc_feature 1..63: note = >NSP12(ORF1b)_680-700 SEQ ID NO:64|descr=Homo sapiens|len=63
  source 1..63: mol_type = other RNA
acctcatcaggagatgccacaactgcttatgctaatagtgtttttaacatttgtcaagctgtc

SEQ ID NO: 65 - RNA, 63 nt, synthetic construct
  misc_feature 1..63: note = >NSP12(ORF1b)_852-872 SEQ ID NO:65|descr=Homo sapiens|len=63
  source 1..63: mol_type = other RNA
ggtacacttatgattgaacggttcgtgtctttagctatagatgcttacccacttactaaacat

SEQ ID NO: 66 - RNA, 63 nt, synthetic construct
  misc_feature 1..63: note = >NSP12(ORF1b)_886-906 SEQ ID NO:66|descr=Homo sapiens|len=63
  source 1..63: mol_type = other RNA
caatacataagaaagctacatgatgagttaacaggacacatgttagacatgtattctgttatg

SEQ ID NO: 67 - RNA, 63 nt, synthetic construct
  misc_feature 1..63: note = >NSP13(ORF1b)_131-151 SEQ ID NO:67|descr=Homo sapiens|len=63
  source 1..63: mol_type = other RNA
aagctttttgcagcagaaacgctcaaagctactgaggagacatttaaactgtcttatggtatt

SEQ ID NO: 68 - RNA, 63 nt, synthetic construct
  misc_feature 1..63: note = >NSP13(ORF1b)_228-248 SEQ ID NO:68|descr=Homo sapiens|len=63
  source 1..63: mol_type = other RNA
acatcacatacagtaatgccattaagtgcacctacactagtgccacaagagcactatgttaga

SEQ ID NO: 69 - RNA, 63 nt, synthetic construct
  misc_feature 1..63: note = >NSP13(ORF1b)_372-392 SEQ ID NO:69|descr=Homo sapiens|len=63
  source 1..63: mol_type = other RNA
gtctttgatgaaatttcaatggccacaaattatgatttgagtgttgtcaatgccagattacgt

SEQ ID NO: 70 - RNA, 63 nt, synthetic construct
  misc_feature 1..63: note = >NSP13(ORF1b)_394-414 SEQ ID NO:70|descr=Homo sapiens|len=63
  source 1..63: mol_type = other RNA
aagcactatgtgtacattggcgaccctgctcaattacctgcaccacgcacattgctaactaag

SEQ ID NO: 71 - RNA, 63 nt, synthetic construct
  misc_feature 1..63: note = >NSP13(ORF1b)_522-542 SEQ ID NO:71|descr=Homo sapiens|len=63
  source 1..63: mol_type = other RNA
gcctcaaagattttgggactaccaactcaaactgttgattcatcacagggctcagaatatgac

SEQ ID NO: 72 - RNA, 63 nt, synthetic construct
  misc_feature 1..63: note = >NSP14(ORF1b)_175-195 SEQ ID NO:72|descr=Homo sapiens|len=63
  source 1..63: mol_type = other RNA
aaaaatctctctgacagagtcgtatttgtcttatgggcacatggctttgagttgacatctatg

SEQ ID NO: 73 - RNA, 63 nt, synthetic construct
  misc_feature 1..63: note = >NSP14(ORF1b)_228-248 SEQ ID NO:73|descr=Homo sapiens|len=63
  source 1..63: mol_type = other RNA
catcattctattggatttgattacgtctataatccgtttatgattgatgttcaacaatggggt

SEQ ID NO: 74 - RNA, 63 nt, synthetic construct
  misc_feature 1..63: note = >NSP14(ORF1b)_320-430 SEQ ID NO:74|descr=Homo sapiens|len=63
  source 1..63: mol_type = other RNA
gcattattagcagacaaattcccagttcttcacgacattggtaaccctaaagctattaagtgt

SEQ ID NO: 75 - RNA, 63 nt, synthetic construct
  misc_feature 1..63: note = >NSP14(ORF1b)_458-478 SEQ ID NO:75|descr=Homo sapiens|len=63
  source 1..63: mol_type = other RNA
caagtagtgtcagatatagattatgtaccactaaagtctgctacgtgtataacacgttgcaat

SEQ ID NO: 76 - RNA, 63 nt, synthetic construct
  misc_feature 1..63: note = >NSP14(ORF1b)_500-520 SEQ ID NO:76|descr=Homo sapiens|len=63
  source 1..63: mol_type = other RNA
atgatgatctcagctggctttagcttgtgggtttacaaacaatttgatacttataacctctgg

SEQ ID NO: 77 - RNA, 63 nt, synthetic construct
  misc_feature 1..63: note = >NSP14(ORF1b)_504-524 SEQ ID NO:77|descr=Homo sapiens|len=63
  source 1..63: mol_type = other RNA
gctggctttagcttgtgggtttacaaacaatttgatacttataacctctggaacacttttaca

SEQ ID NO: 78 - RNA, 63 nt, synthetic construct
  misc_feature 1..63: note = >NSP15(ORF1b)_292-312 SEQ ID NO:78|descr=Homo sapiens|len=63
  source 1..63: mol_type = other RNA
tgttctgttattgatttattacttgatgattttgttgaaataataaaatcccaagatttatct

SEQ ID NO: 79 - RNA, 63 nt, synthetic construct
  misc_feature 1..63: note = >NSP15(ORF1b)_41-61 SEQ ID NO:79|descr=Homo sapiens|len=63
  source 1..63: mol_type = other RNA
gaattgtttgaaaataaaacaacattacctgttaatgtagcatttgagctttgggctaagcgc

SEQ ID NO: 80 - RNA, 63 nt, synthetic construct
  misc_feature 1..63: note = >NSP16(ORF1b)_53-73 SEQ ID NO:80|descr=Homo sapiens|len=63
  source 1..63: mol_type = other RNA
tatttaaacacattaacattagctgtaccctataatatgagagttatacattttggtgctggt

SEQ ID NO: 81 - RNA, 63 nt, synthetic construct
  misc_feature 1..63: note = >NSP3(ORF1a)_1-21 SEQ ID NO:81|descr=Homo sapiens|len=63
  source 1..63: mol_type = other RNA
gcaccaacaaaggttacttttggtgatgacactgtgatagaagtgcaaggttacaagagtgtg

SEQ ID NO: 82 - RNA, 63 nt, synthetic construct
  misc_feature 1..63: note = >NSP3(ORF1a)_1136-1156 SEQ ID NO:82|descr=Homo sapiens|len=63
  source 1..63: mol_type = other RNA
gagcttaaagttacatttttccctgacttaaatggtgatgtggtggctattgattataaacac

SEQ ID NO: 83 - RNA, 63 nt, synthetic construct
  misc_feature 1..63: note = >NSP3(ORF1a)_1504-1524 SEQ ID NO:83|descr=Homo sapiens|len=63
  source 1..63: mol_type = other RNA
ggcttagttgcagagtggtttttggcatatattcttttcactaggtttttctatgtacttgga

SEQ ID NO: 84 - RNA, 63 nt, synthetic construct
  misc_feature 1..63: note = >NSP3(ORF1a)_1549-1569 SEQ ID NO:84|descr=Homo sapiens|len=63
  source 1..63: mol_type = other RNA
ttaataattaatcttgtacaaatggccccgatttcagctatggttagaatgtacatcttcttt

SEQ ID NO: 85 - RNA, 63 nt, synthetic construct
  misc_feature 1..63: note = >NSP3(ORF1a)_209-229 SEQ ID NO:85|descr=Homo sapiens|len=63
  source 1..63: mol_type = other RNA
agttttagtggttatttaaaacttactgacaatgtatacattaaaaatgcagacattgtggaa

SEQ ID NO: 86 - RNA, 63 nt, synthetic construct
  misc_feature 1..63: note = >NSP4(ORF1a)_331-351 SEQ ID NO:86|descr=Homo sapiens|len=63
  source 1..63: mol_type = other RNA
ttaacaccagtttactcattcttacctggtgtttattctgttatttacttgtacttgacattt

SEQ ID NO: 87 - RNA, 63 nt, synthetic construct
  misc_feature 1..63: note = >NSP4(ORF1a)_340-360 SEQ ID NO:87|descr=Homo sapiens|len=63
  source 1..63: mol_type = other RNA
ggtgtttattctgttatttacttgtacttgacattttatcttactaatgatgtttctttttta

SEQ ID NO: 88 - RNA, 63 nt, synthetic construct
  misc_feature 1..63: note = >NSP4(ORF1a)_36-56 SEQ ID NO:88|descr=Homo sapiens|len=63
  source 1..63: mol_type = other RNA
catactgacttttcaagtgaaatcataggatacaaggctattgatggtggtgtcactcgtgac

SEQ ID NO: 89 - RNA, 63 nt, synthetic construct
  misc_feature 1..63: note = >NSP4(ORF1a)_417-437 SEQ ID NO:89|descr=Homo sapiens|len=63
  source 1..63: mol_type = other RNA
ctgtgcacctttttgttaaataaagaaatgtatctaaagttgcgtagtgatgtgctattacct

SEQ ID NO: 90 - RNA, 63 nt, synthetic construct
  misc_feature 1..63: note = >NSP5(ORF1a)_164-184 SEQ ID NO:90|descr=Homo sapiens|len=63
  source 1..63: mol_type = other RNA
catatggaattaccaactggagttcatgctggcacagacttagaaggtaacttttatggacct

SEQ ID NO: 91 - RNA, 72 nt, synthetic construct
  misc_feature 1..72: note = >NSP6(ORF1a)_237-260 SEQ ID NO:91|descr=Homo sapiens|len=72
  source 1..72: mol_type = other RNA
ctgactcttggtgtttatgattacttagtttctacacaggagtttagatatatgaattcacagggactactc

SEQ ID NO: 92 - RNA, 105 nt, synthetic construct
  misc_feature 1..105: note = >NSP8(ORF1a)_150-184 SEQ ID NO:92|descr=Homo sapiens|len=105
  source 1..105: mol_type = other RNA
gcatcagcattgtgggaaatccaacaggttgtagatgcagatagtaaaattgttcaacttagtgaaattagtatggacaattcacctaatttagcatggcctctt

SEQ ID NO: 93 - RNA, 63 nt, synthetic construct
  misc_feature 1..63: note = >NSP8(ORF1a)_53-73 SEQ ID NO:93|descr=Homo sapiens|len=63
  source 1..63: mol_type = other RNA
gcagccatgcaacgtaagttggaaaagatggctgatcaagctatgacccaaatgtataaacag

SEQ ID NO: 94 - RNA, 63 nt, synthetic construct
  misc_feature 1..63: note = >NSP9(ORF1a)_14-34 SEQ ID NO:94|descr=Homo sapiens|len=63
  source 1..63: mol_type = other RNA
tgtgctgccggtactacacaaactgcttgcactgatgacaatgcgttagcttactacaacaca

SEQ ID NO: 95 - RNA, 1449 nt, synthetic construct
  misc_feature 1..1449: note = SARS-CoV-2 construct 2.1_DCL
  source 1..1449: mol_type = other RNA
atggccgctccaggtagtgcaaggagaccacttctccttttgctgctgcttcttttgctcggcctgatgcattgcgctagtgcagccatgtttcatctcgttgactttcaggttactatagcagagatattactaattattatgaggactctgtgcacctttttgttaaataaagaaatgtatctaaagttgcgtagtgatgtgctattacctatatcacgtcaacgtcttactaaatacacaatggcagacctcgtctatgctttaaggcattttactgattacaaacattggccgcaaattgcacaatttgcccccagcgcttcagcgttcttcggagacatcaaggacctgcctaaagaaatcactgttgctacatcacgaacgctttcttattacaaagcatcagcattgtgggaaatccaacaggttgtagatgcagatagtaaaattgttcaacttagtgaaattagtatggacaattcacctaatttagcatggcctcttaagcactatgtgtacattggcgaccctgctcaattacctgcaccacgcacattgctaactaaggcctcaaagattttgggactaccaactcaaactgttgattcatcacagggctcagaatatgacatgtactcattcgtttcggaagagacaggtacgttaatagttaatagcgtacttctttttcttgcattattagcagacaaattcccagttcttcacgacattggtaaccctaaagctattaagtgtgaattgtttgaaaataaaacaacattacctgttaatgtagcatttgagctttgggctaagcgcgctggctttagcttgtgggtttacaaacaatttgatacttataacctctggaacacttttacaggtgtttattctgttatttacttgtacttgacattttatcttactaatgatgtttcttttttaggtacacttatgattgaacggttcgtgtctttagctatagatgcttacccacttactaaacataaaatatttgttgatggtgttccatttgtagtttcaactggataccacttcagagagctaggtcatactgacttttcaagtgaaatcataggatacaaggctattgatggtggtgtcactcgtgacaaaaatctctctgacagagtcgtatttgtcttatgggcacatggctttgagttgacatctatgtcagctggttttccatttaataaatggggtaaggctagactttattatgattcaatgagttatgcaccaacaaaggttacttttggtgatgacactgtgatagaagtgcaaggttacaagagtgtgtcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 96 - RNA, 1953 nt, synthetic construct
  misc_feature 1..1953: note = SARS-CoV-2 construct 2.2_DCL
  source 1..1953: mol_type = other RNA
atggccgctccaggtagtgcaaggagaccacttctccttttgctgctgcttcttttgctcggcctgatgcattgcgctagtgcagccatgtttcatctcgttgactttcaggttactatagcagagatattactaattattatgaggactggtacacttatgattgaacggttcgtgtctttagctatagatgcttacccacttactaaacataaaatatttgttgatggtgttccatttgtagtttcaactggataccacttcagagagctaggtatgatgatctcagctggctttagcttgtgggtttacaaacaatttgatacttataacctctggggcttagttgcagagtggtttttggcatatattcttttcactaggtttttctatgtacttggatcagctggttttccatttaataaatggggtaaggctagactttattatgattcaatgagttattgtgctgccggtactacacaaactgcttgcactgatgacaatgcgttagcttactacaacacagcctcaaagattttgggactaccaactcaaactgttgattcatcacagggctcagaatatgacgacatcaaggacctgcctaaagaaatcactgttgctacatcacgaacgctttcttattacaaagcattattagcagacaaattcccagttcttcacgacattggtaaccctaaagctattaagtgtgaattgtttgaaaataaaacaacattacctgttaatgtagcatttgagctttgggctaagcgcggtgtttattctgttatttacttgtacttgacattttatcttactaatgatgtttcttttttacatactgacttttcaagtgaaatcataggatacaaggctattgatggtggtgtcactcgtgacctgtgcacctttttgttaaataaagaaatgtatctaaagttgcgtagtgatgtgctattacctatgtactcattcgtttcggaagagacaggtacgttaatagttaatagcgtacttctttttcttgttcctgttgtagattcttattattcattgttaatgcctatattaaccttgaccagggctttaacatcacatacagtaatgccattaagtgcacctacactagtgccacaagagcactatgttagagcatcagcattgtgggaaatccaacaggttgtagatgcagatagtaaaattgttcaacttagtgaaattagtatggacaattcacctaatttagcatggcctcttattaccgttgaagagcttaaaaagctccttgaacaatggaacctagtaataggtttcctattcagtccaagatggtatttctactacctaggaactgggccagaagctggacttccctatggtgctatatcacgtcaacgtcttactaaatacacaatggcagacctcgtctatgctttaaggcattttagttttagtggttatttaaaacttactgacaatgtatacattaaaaatgcagacattgtggaagcaccaacaaaggttacttttggtgatgacactgtgatagaagtgcaaggttacaagagtgtgactgattacaaacattggccgcaaattgcacaatttgcccccagcgcttcagcgttcttcggaaagctttttgcagcagaaacgctcaaagctactgaggagacatttaaactgtcttatggtattaaaaatctctctgacagagtcgtatttgtcttatgggcacatggctttgagttgacatctatgaagcactatgtgtacattggcgaccctgctcaattacctgcaccacgcacattgctaactaagtcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 97 - RNA, 1029 nt, synthetic construct
  misc_feature 1..1029: note = SARS-CoV-2 construct 2.2.1_DCL
  source 1..1029: mol_type = other RNA
atggccgctccaggtagtgcaaggagaccacttctccttttgctgctgcttcttttgctcggcctgatgcattgcgctagtgcagccatgtttcatctcgttgactttcaggttactatagcagagatattactaattattatgaggactggtacacttatgattgaacggttcgtgtctttagctatagatgcttacccacttactaaacataaaatatttgttgatggtgttccatttgtagtttcaactggataccacttcagagagctaggtatgatgatctcagctggctttagcttgtgggtttacaaacaatttgatacttataacctctggggcttagttgcagagtggtttttggcatatattcttttcactaggtttttctatgtacttggatcagctggttttccatttaataaatggggtaaggctagactttattatgattcaatgagttattgtgctgccggtactacacaaactgcttgcactgatgacaatgcgttagcttactacaacacagcctcaaagattttgggactaccaactcaaactgttgattcatcacagggctcagaatatgacgacatcaaggacctgcctaaagaaatcactgttgctacatcacgaacgctttcttattacaaagcattattagcagacaaattcccagttcttcacgacattggtaaccctaaagctattaagtgtgaattgtttgaaaataaaacaacattacctgttaatgtagcatttgagctttgggctaagcgcggtgtttattctgttatttacttgtacttgacattttatcttactaatgatgtttcttttttacatactgacttttcaagtgaaatcataggatacaaggctattgatggtggtgtcactcgtgactcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 98 - RNA, 1134 nt, synthetic construct
  misc_feature 1..1134: note = SARS-CoV-2 construct 2.2.2_DCL
  source 1..1134: mol_type = other RNA
atggccgctccaggtagtgcaaggagaccacttctccttttgctgctgcttcttttgctcggcctgatgcattgcgctagtgcagccctgtgcacctttttgttaaataaagaaatgtatctaaagttgcgtagtgatgtgctattacctatgtactcattcgtttcggaagagacaggtacgttaatagttaatagcgtacttctttttcttgttcctgttgtagattcttattattcattgttaatgcctatattaaccttgaccagggctttaacatcacatacagtaatgccattaagtgcacctacactagtgccacaagagcactatgttagagcatcagcattgtgggaaatccaacaggttgtagatgcagatagtaaaattgttcaacttagtgaaattagtatggacaattcacctaatttagcatggcctcttattaccgttgaagagcttaaaaagctccttgaacaatggaacctagtaataggtttcctattcagtccaagatggtatttctactacctaggaactgggccagaagctggacttccctatggtgctatatcacgtcaacgtcttactaaatacacaatggcagacctcgtctatgctttaaggcattttagttttagtggttatttaaaacttactgacaatgtatacattaaaaatgcagacattgtggaagcaccaacaaaggttacttttggtgatgacactgtgatagaagtgcaaggttacaagagtgtgactgattacaaacattggccgcaaattgcacaatttgcccccagcgcttcagcgttcttcggaaagctttttgcagcagaaacgctcaaagctactgaggagacatttaaactgtcttatggtattaaaaatctctctgacagagtcgtatttgtcttatgggcacatggctttgagttgacatctatgaagcactatgtgtacattggcgaccctgctcaattacctgcaccacgcacattgctaactaagtcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 99 - RNA, 1479 nt, synthetic construct
  misc_feature 1..1479: note = SARS-CoV-2 construct 2.3_DCL
  source 1..1479: mol_type = other RNA
atggccgctccaggtagtgcaaggagaccacttctccttttgctgctgcttcttttgctcggcctgatgcattgcgctagtgcagcctcagctggttttccatttaataaatggggtaaggctagactttattatgattcaatgagttataatttaattgattcttactttgtagttaagagacacactttctctaactaccaacatgaagaaccgcaaattgcacaatttgcccccagcgcttcagcgttcttcggaatgtcgcgcattggcatggacatcaaggacctgcctaaagaaatcactgttgctacatcacgaacgctttcttattacaaattaataattaatcttgtacaaatggccccgatttcagctatggttagaatgtacatcttctttctgtgcacctttttgttaaataaagaaatgtatctaaagttgcgtagtgatgtgctattacctgcctcaaagattttgggactaccaactcaaactgttgattcatcacagggctcagaatatgacatatcacgtcaacgtcttactaaatacacaatggcagacctcgtctatgctttaaggcattttggtacacttatgattgaacggttcgtgtctttagctatagatgcttacccacttactaaacatctgactcttggtgtttatgattacttagtttctacacaggagtttagatatatgaattcacagggactactcatgtactcattcgtttcggaagagacaggtacgttaatagttaatagcgtacttctttttcttggcttagtagaagttgaaaaaggcgttttgcctcaacttgaacagccctatgtgttcatcaaacaatacataagaaagctacatgatgagttaacaggacacatgttagacatgtattctgttatgtatttaaacacattaacattagctgtaccctataatatgagagttatacattttggtgctggtgaattgtttgaaaataaaacaacattacctgttaatgtagcatttgagctttgggctaagcgcaagctttttgcagcagaaacgctcaaagctactgaggagacatttaaactgtcttatggtatttgttctgttattgatttattacttgatgattttgttgaaataataaaatcccaagatttatctcaagtagtgtcagatatagattatgtaccactaaagtctgctacgtgtataacacgttgcaataaaatatttgttgatggtgttccatttgtagtttcaactggataccacttcagagagctaggtgagcttaaagttacatttttccctgacttaaatggtgatgtggtggctattgattataaacactcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 100 - RNA, 2046 nt, synthetic construct
  misc_feature 1..2046: note = SARS-CoV-2 construct 2.4_DCL
  source 1..2046: mol_type = other RNA
atggccgctccaggtagtgcaaggagaccacttctccttttgctgctgcttcttttgctcggcctgatgcattgcgctagtgcagccctgactcttggtgtttatgattacttagtttctacacaggagtttagatatatgaattcacagggactactcgcctcaaagattttgggactaccaactcaaactgttgattcatcacagggctcagaatatgactgttctgttattgatttattacttgatgattttgttgaaataataaaatcccaagatttatctcaatacataagaaagctacatgatgagttaacaggacacatgttagacatgtattctgttatgttaacaccagtttactcattcttacctggtgtttattctgttatttacttgtacttgacatttaatttaattgattcttactttgtagttaagagacacactttctctaactaccaacatgaagaatcagctggttttccatttaataaatggggtaaggctagactttattatgattcaatgagttattatttaaacacattaacattagctgtaccctataatatgagagttatacattttggtgctggtcatatggaattaccaactggagttcatgctggcacagacttagaaggtaacttttatggacctcaagtagtgtcagatatagattatgtaccactaaagtctgctacgtgtataacacgttgcaatcatcattctattggatttgattacgtctataatccgtttatgattgatgttcaacaatggggtccgcaaattgcacaatttgcccccagcgcttcagcgttcttcggaatgtcgcgcattggcatgaagctttttgcagcagaaacgctcaaagctactgaggagacatttaaactgtcttatggtattggtgtctctatctgtagtactatgaccaatagacagtttcatcaaaaattattgaaatcaatactgtgcacctttttgttaaataaagaaatgtatctaaagttgcgtagtgatgtgctattacctatgtactcattcgtttcggaagagacaggtacgttaatagttaatagcgtacttctttttcttggtacacttatgattgaacggttcgtgtctttagctatagatgcttacccacttactaaacatgaattgtttgaaaataaaacaacattacctgttaatgtagcatttgagctttgggctaagcgcggttgggattatcctaaatgtgatagagccatgcctaacatgcttagaattatggcctcacttatatcacgtcaacgtcttactaaatacacaatggcagacctcgtctatgctttaaggcatttttttgcctatgccaacaggaataggtttttgtatataattaagttaattttcctctggctgttagcagccatgcaacgtaagttggaaaagatggctgatcaagctatgacccaaatgtataaacagttaataattaatcttgtacaaatggccccgatttcagctatggttagaatgtacatcttctttgacatcaaggacctgcctaaagaaatcactgttgctacatcacgaacgctttcttattacaaaaaaatatttgttgatggtgttccatttgtagtttcaactggataccacttcagagagctaggtacctcatcaggagatgccacaactgcttatgctaatagtgtttttaacatttgtcaagctgtcgtctttgatgaaatttcaatggccacaaattatgatttgagtgttgtcaatgccagattacgtgagcttaaagttacatttttccctgacttaaatggtgatgtggtggctattgattataaacacggcttagtagaagttgaaaaaggcgttttgcctcaacttgaacagccctatgtgttcatcaaatcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 101 - RNA, 1101 nt, synthetic construct
  misc_feature 1..1101: note = SARS-CoV-2 construct 4.1_DCL
  source 1..1101: mol_type = other RNA
atggccgctccaggtagtgcaaggagaccacttctccttttgctgctgcttcttttgctcggcctgatgcattgcgctagtgcagccctgactcttggtgtttatgattacttagtttctacacaggagtttagatatatgaattcacagggactactcgcctcaaagattttgggactaccaactcaaactgttgattcatcacagggctcagaatatgactgttctgttattgatttattacttgatgattttgttgaaataataaaatcccaagatttatctcaatacataagaaagctacatgatgagttaacaggacacatgttagacatgtattctgttatgttaacaccagtttactcattcttacctggtgtttattctgttatttacttgtacttgacatttaatttaattgattcttactttgtagttaagagacacactttctctaactaccaacatgaagaatcagctggttttccatttaataaatggggtaaggctagactttattatgattcaatgagttattatttaaacacattaacattagctgtaccctataatatgagagttatacattttggtgctggtcatatggaattaccaactggagttcatgctggcacagacttagaaggtaacttttatggacctcaagtagtgtcagatatagattatgtaccactaaagtctgctacgtgtataacacgttgcaatcatcattctattggatttgattacgtctataatccgtttatgattgatgttcaacaatggggtccgcaaattgcacaatttgcccccagcgcttcagcgttcttcggaatgtcgcgcattggcatgaagctttttgcagcagaaacgctcaaagctactgaggagacatttaaactgtcttatggtattggtgtctctatctgtagtactatgaccaatagacagtttcatcaaaaattattgaaatcaatatcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 102 - RNA, 1155 nt, synthetic construct
  misc_feature 1..1155: note = SARS-CoV-2 construct 2.4.2_DCL
  source 1..1155: mol_type = other RNA
atggccgctccaggtagtgcaaggagaccacttctccttttgctgctgcttcttttgctcggcctgatgcattgcgctagtgcagccctgtgcacctttttgttaaataaagaaatgtatctaaagttgcgtagtgatgtgctattacctatgtactcattcgtttcggaagagacaggtacgttaatagttaatagcgtacttctttttcttggtacacttatgattgaacggttcgtgtctttagctatagatgcttacccacttactaaacatgaattgtttgaaaataaaacaacattacctgttaatgtagcatttgagctttgggctaagcgcggttgggattatcctaaatgtgatagagccatgcctaacatgcttagaattatggcctcacttatatcacgtcaacgtcttactaaatacacaatggcagacctcgtctatgctttaaggcatttttttgcctatgccaacaggaataggtttttgtatataattaagttaattttcctctggctgttagcagccatgcaacgtaagttggaaaagatggctgatcaagctatgacccaaatgtataaacagttaataattaatcttgtacaaatggccccgatttcagctatggttagaatgtacatcttctttgacatcaaggacctgcctaaagaaatcactgttgctacatcacgaacgctttcttattacaaaaaaatatttgttgatggtgttccatttgtagtttcaactggataccacttcagagagctaggtacctcatcaggagatgccacaactgcttatgctaatagtgtttttaacatttgtcaagctgtcgtctttgatgaaatttcaatggccacaaattatgatttgagtgttgtcaatgccagattacgtgagcttaaagttacatttttccctgacttaaatggtgatgtggtggctattgattataaacacggcttagtagaagttgaaaaaggcgttttgcctcaacttgaacagccctatgtgttcatcaaatcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 103 - RNA, 840 nt, synthetic construct
  misc_feature 1..840: note = SARS-CoV-2 construct 2.5_DCL
  source 1..840: mol_type = other RNA
atggccgctccaggtagtgcaaggagaccacttctccttttgctgctgcttcttttgctcggcctgatgcattgcgctagtgcagccctgtgcacctttttgttaaataaagaaatgtatctaaagttgcgtagtgatgtgctattacctaaaatatttgttgatggtgttccatttgtagtttcaactggataccacttcagagagctaggttcagctggttttccatttaataaatggggtaaggctagactttattatgattcaatgagttatgacatcaaggacctgcctaaagaaatcactgttgctacatcacgaacgctttcttattacaaaggtacacttatgattgaacggttcgtgtctttagctatagatgcttacccacttactaaacatgaattgtttgaaaataaaacaacattacctgttaatgtagcatttgagctttgggctaagcgcgcctcaaagattttgggactaccaactcaaactgttgattcatcacagggctcagaatatgacaagctttttgcagcagaaacgctcaaagctactgaggagacatttaaactgtcttatggtattatatcacgtcaacgtcttactaaatacacaatggcagacctcgtctatgctttaaggcattttatgtactcattcgtttcggaagagacaggtacgttaatagttaatagcgtacttctttttctttcatctgattatacgattgtgctgccagtgattggcgcaatcgtagttggactgtgtctgatgggcatgggggtatacaaaatcaggttgcgctgtcagtcttccggttaccaacgcatttga

SEQ ID NO: 104 - RNA, 1449 nt, synthetic construct
  misc_feature 1..1449: note = SARS-CoV-2_concat_2.1_DCL_opt1
  source 1..1449: mol_type = other RNA
atggctgctcctggatctgctagaaggcctctgctgctgcttctgttgctcctgctgctgggcctgatgcattgtgcctctgccgccatgttccacctggtggacttccaagtgacaatcgccgagatcctgctgatcatcatgcggaccctgtgcaccttcctgctgaacaaagagatgtacctgaagctgcggagcgacgtgctgctgcctatcagcagacagcggctgaccaagtacaccatggccgatctggtgtacgccctgagacacttcaccgactacaagcactggccccagatcgcccagtttgccccttctgccagcgccttcttcggcgacattaaggacctccctaaagaaatcaccgtggccaccagcagaaccctgagctactacaaggctagcgccctgtgggagatccagcaggttgtggatgccgacagcaagatcgtgcagctgagcgagatcagcatggacaacagccccaatctggcctggcctctgaagcactacgtgtacatcggcgaccctgcacagctgcccgctcctagaacactgctgacaaaggcctccaagatcctgggcctgcctacacagaccgtggatagctctcagggcagcgagtacgacatgtacagcttcgtgtccgaggaaaccggcacactgatcgtgaactctgtcctgctgtttctggccctgctggccgacaagtttcctgtgctgcacgacatcggcaaccccaaggccattaagtgcgagctgttcgagaacaagaccacactgcccgtgaacgtggcctttgagctgtgggccaaacgcgccggattttccctgtgggtgtacaagcagttcgacacctacaacctgtggaacaccttcaccggcgtgtacagcgtgatctacctgtacctgaccttctacctgaccaacgacgtgtccttcctgggcaccctgatgatcgagagattcgtgtccctggctatcgacgcctatcctctgaccaagcacaagatcttcgtggacggcgtgcccttcgtggtgtctaccggctaccactttagagagctgggccacaccgacttcagctccgagatcatcggctacaaggccatcgacggcggcgtgaccagagacaagaacctgagcgacagagtggtgttcgtgctgtgggctcacggattcgagctgacctctatgagcgccggctttcccttcaacaaatggggcaaagcccggctgtactacgacagcatgagctacgcccctaccaaagtgacctttggcgacgacaccgtgatcgaggtgcagggctacaagagcgtgtccagcgactacaccatcgtgctgcctgtgatcggcgccatcgttgtgggactgtgtctgatgggcatgggagtgtacaagatccggctgagatgccagtccagcggctaccagagaatctga

SEQ ID NO: 105 - RNA, 1449 nt, synthetic construct
  misc_feature 1..1449: note = SARS-CoV-2_concat_2.1_DCL_opt2
  source 1..1449: mol_type = other RNA
atggccgcccccggcagcgcccgccgccccctgctgctgctgctgctgctgctgctgctgggcctgatgcactgcgccagcgccgccatgttccacctggtggacttccaggtgaccatcgccgagatcctgctgatcatcatgcgcaccctgtgcaccttcctgctgaacaaggagatgtacctgaagctgcgcagcgacgtgctgctgcccatcagccgccagcgcctgaccaagtacaccatggccgacctggtgtacgccctgcgccacttcaccgactacaagcactggccccagatcgcccagttcgcccccagcgccagcgccttcttcggcgacatcaaggacctccccaaggagatcaccgtggccaccagccgcaccctgagctactacaaggccagcgccctgtgggagatccagcaggtggtggacgccgacagcaagatcgtgcagctgagcgagatcagcatggacaacagccccaacctggcctggcccctgaagcactacgtgtacatcggcgaccccgcccagctgcccgccccccgcaccctgctgaccaaggccagcaagatcctgggcctgcccacccagaccgtggacagcagccagggcagcgagtacgacatgtacagcttcgtgagcgaggagaccggcaccctgatcgtgaacagcgtgctgctgttcctggccctgctggccgacaagttccccgtgctgcacgacatcggcaaccccaaggccatcaagtgcgagctgttcgagaacaagaccaccctgcccgtgaacgtggccttcgagctgtgggccaagcgcgccggcttcagcctgtgggtgtacaagcagttcgacacctacaacctgtggaacaccttcaccggcgtgtacagcgtgatctacctgtacctgaccttctacctgaccaacgacgtgagcttcctgggcaccctgatgatcgagcgcttcgtgagcctggccatcgacgcctaccccctgaccaagcacaagatcttcgtggacggcgtgcccttcgtggtgagcaccggctaccacttccgcgagctgggccacaccgacttcagcagcgagatcatcggctacaaggccatcgacggcggcgtgacccgcgacaagaacctgagcgaccgcgtggtgttcgtgctgtgggcccacggcttcgagctgaccagcatgagcgccggcttccccttcaacaagtggggcaaggcccgcctgtactacgacagcatgagctacgcccccaccaaggtgaccttcggcgacgacaccgtgatcgaggtgcagggctacaagagcgtgagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 106 - RNA, 1449 nt, synthetic construct
  misc_feature 1..1449: note = SARS-CoV-2_concat_2.1_DCL_opt3
  source 1..1449: mol_type = other RNA
atggctgccccaggctctgcaaggcggcctctgctgctgctgttgctgctgctgctgctgggactgatgcactgcgcatctgccgccatgttccacctggtggacttccaggtgacaatcgctgagatcttgctgatcatcatgagaaccctgtgcaccttcctgctgaacaaggagatgtacctgaagctgaggagcgacgtgctgctccctatttccagacagagactgacaaaatatacaatggccgatctggtgtacgccctgcggcattttaccgattacaagcactggccccagatcgcccagttcgcccctagcgccagcgcctttttcggggacattaaggacctccctaaagagatcactgtggccacctcaaggaccctgtcttactacaaggccagcgcactgtgggaaattcagcaggtggtggacgccgactctaagatcgtgcagctgtccgagattagcatggataattcacccaatctggcctggcccctgaaacactacgtgtacatcggcgatcccgcccagctgcccgccccccgcacccttctgacaaaagcttctaagatcctgggcctgcctacacagactgtggacagctcacaggggagcgagtacgatatgtactctttcgtgagcgaggagacaggcaccctgattgtgaacagcgtgctgctgttcctggccctgctggccgataagtttcccgtgctgcacgacattggcaaccccaaggccatcaaatgcgaactgttcgagaacaagaccactctgcccgtgaatgtggcctttgagctgtgggctaaaagagccggcttctccctgtgggtgtacaagcagtttgacacctacaacctgtggaacacctttacaggagtctactccgtgatctacctgtacctgaccttctatctgacaaacgacgtgagctttctgggaacactgatgatcgagagattcgtgagcctggcgatcgacgcctaccccctgaccaagcacaaaattttcgtcgacggggtgcccttcgtggtgtccacaggctaccacttcagagaactgggccacacagatttttccagtgagattatcggctacaaggctattgacggcggagtgacaagagataagaatctgtctgatagggtggtgttcgtgctgtgggcccacggatttgagctgacctctatgtcagccggattcccttttaacaaatggggcaaggctcggctgtactacgactcaatgtcctacgcccccaccaaggtgacattcggcgatgacacagtgatcgaggtgcagggatacaagtccgtgtctagcgactatacaattgtgctccctgtgatcggcgccatcgtggtggggctgtgcctcatgggcatgggcgtgtataaaatccggctgagatgtcagagcagcggctatcagcgtatctag

SEQ ID NO: 107 - RNA, 1953 nt, synthetic construct
  misc_feature 1..1953: note = SARS-CoV-2_concat_2.2_DCL_opt1
  source 1..1953: mol_type = other RNA
atggctgctcctggatctgctagaaggcctctgctgctgcttctgttgctcctgctgctgggcctgatgcattgtgcctctgccgccatgttccacctggtggacttccaagtgacaatcgccgagatcctgctgatcatcatgcggaccggcacactgatgatcgagagattcgtgtccctggctatcgacgcctatcctctgaccaagcacaagatcttcgtggacggcgtgcccttcgtggtgtctaccggctaccacttcagagaactgggaatgatgatctccgccggcttcagcctgtgggtgtacaagcagttcgacacctacaacctgtggggcctcgtggccgaatggttcctggcctacatcctgttcacccggttcttctacgtgctgggctctgccggctttcccttcaacaaatggggcaaagcccggctgtactacgacagcatgagctactgtgccgccggaaccacacagaccgcctgcacagatgataacgccctggcctattacaacaccgccagcaagatcctgggcctgcctacacagacagtggatagctctcagggcagcgagtacgacgacatcaaggacctccctaaagaaatcaccgtggccaccagcagaaccctgagctactataaggccctgctggccgacaagttccccgtgctgcacgatatcggcaaccccaaggccatcaagtgcgagctgttcgagaacaagaccacactgcccgtgaacgtggcctttgagctgtgggctaaacggggcgtgtacagcgtgatctacctgtacctgaccttctacctgaccaacgacgtgtccttcctgcacaccgacttcagcagcgagatcatcggctacaaggccattgatggcggcgtgaccagagatctgtgcaccttcctgctgaacaaagagatgtacctgaagctgcggagcgacgtgctgctgcccatgtacagctttgtgtccgaggaaacaggcaccctgatcgtgaactctgtcctgctgttcctggtgcctgtggtggacagctactacagcctgctgatgcccatcctgacactgacaagagccctgaccagccacaccgtgatgcctctgtctgctcctacactggtgccccaagagcactatgtgcgggcatctgccctgtgggagatccagcaagtggtggacgccgactccaagattgtgcagctgagcgagatctccatggacaacagccccaatctggcctggcctctgatcacagtggaagaactgaagaaactgctggaacagtggaacctcgtgatcggcttcctgttcagccccagatggtacttctactacctcggcacaggacccgaagccggacttccttatggcgctatctcccggcagcggctgaccaagtataccatggccgatctggtgtacgccctgcggcactttagcttcagcggctatctgaagctgaccgacaacgtgtacatcaagaacgccgacatcgtggaagcccctaccaaagtgaccttcggcgacgataccgtgatcgaggtgcagggctacaagagcgtgaccgactacaagcactggccccagatcgcccagtttgccccttctgccagcgccttcttcggcaagctgtttgccgccgagacactgaaggccaccgaagagacattcaagctgagctacggcatcaagaatctgagcgacagagtggtgttcgtgctgtgggcccatggattcgagctgaccagcatgaagcactacgtctacatcggcgaccccgcacaactgcccgctcctagaacactgctgaccaaaagcagcgactacaccatcgtgctgcctgtgatcggcgccatcgttgtgggactgtgtctgatgggcatgggagtgtacaagatccggctgagatgccagtccagcggctaccagagaatctga

SEQ ID NO: 108 - RNA, 1953 nt, synthetic construct
  misc_feature 1..1953: note = SARS-CoV-2_concat_2.2_DCL_opt2
  source 1..1953: mol_type = other RNA
atggccgcccccggcagcgcccgccgccccctgctgctgctgctgctgctgctgctgctgggcctgatgcactgcgccagcgccgccatgttccacctggtggacttccaggtgaccatcgccgagatcctgctgatcatcatgcgcaccggcaccctgatgatcgagcgcttcgtgagcctggccatcgacgcctaccccctgaccaagcacaagatcttcgtggacggcgtgcccttcgtggtgagcaccggctaccacttccgcgagctgggcatgatgatcagcgccggcttcagcctgtgggtgtacaagcagttcgacacctacaacctgtggggcctggtggccgagtggttcctggcctacatcctgttcacccgcttcttctacgtgctgggcagcgccggcttccccttcaacaagtggggcaaggcccgcctgtactacgacagcatgagctactgcgccgccggcaccacccagaccgcctgcaccgacgacaacgccctggcctactacaacaccgccagcaagatcctgggcctgcccacccagaccgtggacagcagccagggcagcgagtacgacgacatcaaggacctccccaaggagatcaccgtggccaccagccgcaccctgagctactacaaggccctgctggccgacaagttccccgtgctgcacgacatcggcaaccccaaggccatcaagtgcgagctgttcgagaacaagaccaccctgcccgtgaacgtggccttcgagctgtgggccaagcgcggcgtgtacagcgtgatctacctgtacctgaccttctacctgaccaacgacgtgagcttcctgcacaccgacttcagcagcgagatcatcggctacaaggccatcgacggcggcgtgacccgcgacctgtgcaccttcctgctgaacaaggagatgtacctgaagctgcgcagcgacgtgctgctgcccatgtacagcttcgtgagcgaggagaccggcaccctgatcgtgaacagcgtgctgctgttcctggtgcccgtggtggacagctactacagcctgctgatgcccatcctgaccctgacccgcgccctgaccagccacaccgtgatgcccctgagcgcccccaccctggtgccccaggagcactacgtgcgcgccagcgccctgtgggagatccagcaagtggtggacgccgacagcaagatcgtgcagctgagcgagatcagcatggacaacagccccaacctggcctggcccctgatcaccgtggaggagctgaagaagctgctggagcagtggaacctggtgatcggcttcctgttcagcccccgctggtacttctactacctgggcaccggccccgaggccggcctgccctacggcgccatcagccgccagcgcctgaccaagtacaccatggccgacctggtgtacgccctgcgccacttcagcttcagcggctacctgaagctgaccgacaacgtgtacatcaagaacgccgacatcgtggaggcccccaccaaggtgaccttcggcgacgacaccgtgatcgaggtgcagggctacaagagcgtgaccgactacaagcactggccccagatcgcccagttcgcccccagcgccagcgccttcttcggcaagctgttcgccgccgagaccctgaaggccaccgaggagaccttcaagctgagctacggcatcaagaacctgagcgaccgcgtggtgttcgtgctgtgggcccacggcttcgagctgaccagcatgaagcactacgtgtacatcggcgaccccgcccagctgcccgccccccgcaccctgctgaccaagagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 109 - RNA, 1953 nt, synthetic construct
  misc_feature 1..1953: note = SARS-CoV-2_concat_2.2_DCL_opt3
  source 1..1953: mol_type = other RNA
atggccgccccagggagcgcccgcaggcctctgctgctgctgctgctgctgctcctgctgggcctgatgcattgtgccagcgccgccatgttccacctggtggactttcaggtgaccattgccgagatcctgctgatcatcatgcggaccggaactctgatgatcgagaggtttgtgtctctggcaatcgatgcttacccattgaccaaacacaagattttcgtggatggcgtgcccttcgtggtctcaaccggctatcactttagagagctgggaatgatgatctccgccggcttcagcctgtgggtctacaagcagttcgacacttacaatctgtgggggctggtcgccgagtggttcctggcttacatcctgttcacacgcttcttctacgtgctgggctctgccggctttcccttcaataagtggggcaaggctagactgtactatgattccatgagctactgtgcagccggcactacacagaccgcctgcacagacgataatgcactggcctactataacacagcctccaagatcctgggcctgcctactcagaccgtggacagtagtcagggctctgagtacgacgatatcaaggatctgcccaaagagatcacggtggccacatccagaacactcagctactacaaggccctgctggcagataaattcccagtgctgcacgacatcggtaaccctaaggccattaaatgcgagctgttcgaaaacaagacaaccctgcccgtgaacgtggctttcgagctgtgggccaagaggggggtgtattctgtgatctacctgtacctgacattctacctgacaaatgacgtcagcttcctgcacactgatttttccagcgagattatcggctataaggccattgatggcggcgtgacacgcgatctgtgcaccttcctgctgaataaggaaatgtacctgaagctgcgcagcgacgtgctgctgccaatgtacagttttgtgagcgaggagacgggcaccctgatcgtgaacagcgtgctgctgttcctggtgccagtggtggactcatattacagcctgctgatgcctatccttaccctgacacgcgctctgacttctcacaccgtgatgcctctctccgccccaaccctcgtgccacaggagcattatgtgcgggcatcagcactgtgggaaatccagcaagtggtggatgccgacagcaagatcgtgcagctgtccgaaattagcatggataactcacccaatctggcctggcccctgattaccgtggaggagctgaagaagctgctggagcagtggaatctggtgatcgggttcctgttttctcctaggtggtatttttactatctgggcaccggccccgaggccggcctgccttacggagccatcagccgccagcgcctgactaagtataccatggccgatctggtgtacgccctgagacacttctcattcagcggatatctgaagctgaccgacaacgtgtacatcaaaaacgctgatatcgtggaggcccctaccaaggtgaccttcggagacgatacagtgatcgaagtgcaggggtacaagtccgtgaccgattacaagcactggccgcagatcgctcagttcgcccccagcgccagcgccttctttgggaagctgttcgccgccgagaccctgaaggccacagaagagacttttaagctttcttatgggattaaaaacctgagcgaccgggtggtgtttgtgctgtgggcccacggcttcgagctgacctctatgaaacactacgtgtacattggagaccccgcccagctgcctgccccccgcaccttgctgaccaagagcagcgactacaccattgtgctgccagtgataggggccattgtggtgggactctgccttatgggcatgggcgtgtacaagatccggctgcgctgccagtctagcggataccagagaatctga

SEQ ID NO: 110 - RNA, 1029 nt, synthetic construct
  misc_feature 1..1029: note = SARS-CoV-2_concat_2.2.1_DCL_opt1
  source 1..1029: mol_type = other RNA
atggctgctcctggatctgctagaaggcctctgctgctgcttctgttgctcctgctgctgggcctgatgcattgtgcctctgccgccatgttccacctggtggacttccaagtgacaatcgccgagatcctgctgatcatcatgcggaccggcacactgatgatcgagagattcgtgtccctggctatcgacgcctatcctctgaccaagcacaagatcttcgtggacggcgtgcccttcgtggtgtctaccggctaccacttcagagaactgggaatgatgatctccgccggcttcagcctgtgggtgtacaagcagttcgacacctacaacctgtggggcctcgtggccgaatggttcctggcctacatcctgttcacccggttcttctacgtgctgggctctgccggctttcccttcaacaaatggggcaaagcccggctgtactacgacagcatgagctactgtgccgccggaaccacacagaccgcctgcacagatgataacgccctggcctattacaacaccgccagcaagatcctgggcctgcctacacagacagtggatagctctcagggcagcgagtacgacgacatcaaggacctccctaaagaaatcaccgtggccaccagcagaaccctgagctactataaggccctgctggccgacaagttccccgtgctgcacgatatcggcaaccccaaggccatcaagtgcgagctgttcgagaacaagaccacactgcccgtgaacgtggcctttgagctgtgggctaaacggggcgtgtacagcgtgatctacctgtacctgaccttctacctgaccaacgacgtgtccttcctgcacaccgacttcagcagcgagatcatcggctacaaggccattgatggcggcgtgaccagagacagcagcgactacactatcgtgctgcctgtgatcggcgccatcgttgtgggactgtgtctgatgggcatgggagtgtacaagatccggctgagatgccagagcagcggctaccagagaatctga

SEQ ID NO: 111 - RNA, 1029 nt, synthetic construct
  misc_feature 1..1029: note = SARS-CoV-2_concat_2.2.1_DCL_opt2
  source 1..1029: mol_type = other RNA
atggccgcccccggcagcgcccgccgccccctgctgctgctgctgctgctgctgctgctgggcctgatgcactgcgccagcgccgccatgttccacctggtggacttccaggtgaccatcgccgagatcctgctgatcatcatgcgcaccggcaccctgatgatcgagcgcttcgtgagcctggccatcgacgcctaccccctgaccaagcacaagatcttcgtggacggcgtgcccttcgtggtgagcaccggctaccacttccgcgagctgggcatgatgatcagcgccggcttcagcctgtgggtgtacaagcagttcgacacctacaacctgtggggcctggtggccgagtggttcctggcctacatcctgttcacccgcttcttctacgtgctgggcagcgccggcttccccttcaacaagtggggcaaggcccgcctgtactacgacagcatgagctactgcgccgccggcaccacccagaccgcctgcaccgacgacaacgccctggcctactacaacaccgccagcaagatcctgggcctgcccacccagaccgtggacagcagccagggcagcgagtacgacgacatcaaggacctccccaaggagatcaccgtggccaccagccgcaccctgagctactacaaggccctgctggccgacaagttccccgtgctgcacgacatcggcaaccccaaggccatcaagtgcgagctgttcgagaacaagaccaccctgcccgtgaacgtggccttcgagctgtgggccaagcgcggcgtgtacagcgtgatctacctgtacctgaccttctacctgaccaacgacgtgagcttcctgcacaccgacttcagcagcgagatcatcggctacaaggccatcgacggcggcgtgacccgcgacagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 112 - RNA, 1029 nt, synthetic construct
  misc_feature 1..1029: note = SARS-CoV-2_concat_2.2.1_DCL_opt3
  source 1..1029: mol_type = other RNA
atggccgctccaggcagcgcccggcgccctctgctgttgctgctgctgctgctgctgctgggcctgatgcattgcgcctctgccgccatgttccacctggtggacttccaggtgaccattgccgagattctgctgattatcatgaggaccggcacactgatgatcgagagattcgtctctctggccatcgacgcttaccctctgacaaaacacaagatcttcgtggatggggtgcccttcgtggtgtctaccgggtaccacttcagggagctgggaatgatgatctccgccggcttcagcctgtgggtctacaaacagttcgacacatacaacctgtggggcctggtggcagagtggtttctcgcctacatcctgttcaccagattcttctatgtgctgggctcagctggctttccattcaataaatggggcaaagcccggctgtattacgacagcatgagctactgcgctgccggaaccacacagacagcctgcaccgacgataatgccctggcctactacaacaccgctagcaagattctgggcctgcctacacagactgtggatagcagccaggggagcgagtacgacgacatcaaggatctgcctaaggagatcacagtggccaccagcaggaccctgagctactacaaggccctgctggccgataagtttccagtgctgcacgacattggcaaccctaaggccatcaagtgtgaactgttcgaaaacaagaccaccctgcccgtgaacgtggcctttgagctgtgggctaagcgcggcgtgtactccgtgatctatctctatctgaccttttacctgaccaatgacgtgagcttcctgcacaccgacttttcctccgagatcattggctacaaagccatcgatggcggcgtcaccagggacagctctgattacactatcgtgctgcctgtgatcggcgctatcgtggtgggcctgtgtttgatgggcatgggagtgtataagattaggcttaggtgtcagtccagcggctaccagagaatctaa

SEQ ID NO: 113 - RNA, 1134 nt, synthetic construct
  misc_feature 1..1134: note = SARS-CoV-2_concat_2.2.2_DCL_opt1
  source 1..1134: mol_type = other RNA
atggctgctcctggatctgctagaaggcctctgctgctgcttctgttgctcctgctgctgggcctgatgcactgtgcctctgcagccctgtgtaccttcctgctgaacaaagagatgtacctgaagctgcggagcgacgtgctgctgcccatgtacagctttgtgtccgaggaaaccggcacactgatcgtgaactctgtcctgctgttcctggtgcctgtggtggacagctactacagcctgctgatgcccatcctgacactgacaagagccctgaccagccacaccgtgatgcctctgtctgctcctacactggtgccccaagagcactatgtgcgggcatctgccctgtgggagatccagcaagtggtggacgccgacagcaagatcgtgcagctgagcgagatcagcatggacaacagccccaatctggcctggcctctgatcaccgtggaagaactgaagaaactgctggaacagtggaacctcgtgatcggcttcctgttcagccccagatggtacttctactacctcggcacaggacccgaagccggacttccttatggcgctatctcccggcagcggctgaccaagtataccatggccgatctggtgtacgccctgcggcactttagcttcagcggctatctgaagctgaccgacaacgtgtacatcaagaacgccgacatcgtggaagcccctaccaaagtgaccttcggcgacgataccgtgatcgaggtgcagggctacaagagcgtgaccgactacaagcactggccccagatcgcccagtttgccccttctgcctctgccttcttcggcaagctgtttgccgccgagacactgaaggccaccgaagagacattcaagctgagctacggcatcaagaatctgagcgacagagtggtgttcgtgctgtgggcccatggctttgagctgaccagcatgaagcactacgtctacatcggcgaccccgcacaactgcccgctcctagaacactgctgaccaaaagcagcgactacaccatcgtgctgcctgtgatcggcgccatcgttgtgggactgtgtctgatgggcatgggcgtgtacaagatccggctgagatgccagtctagcggctaccagagaatctga

SEQ ID NO: 114 - RNA, 1134 nt, synthetic construct
  misc_feature 1..1134: note = SARS-CoV-2_concat_2.2.2_DCL_opt2
  source 1..1134: mol_type = other RNA
atggccgcccccggcagcgcccgccgccccctgctgctgctgctgctgctgctgctgctgggcctgatgcactgcgccagcgccgccctgtgcaccttcctgctgaacaaggagatgtacctgaagctgcgcagcgacgtgctgctgcccatgtacagcttcgtgagcgaggagaccggcaccctgatcgtgaacagcgtgctgctgttcctggtgcccgtggtggacagctactacagcctgctgatgcccatcctgaccctgacccgcgccctgaccagccacaccgtgatgcccctgagcgcccccaccctggtgccccaggagcactacgtgcgcgccagcgccctgtgggagatccagcaagtggtggacgccgacagcaagatcgtgcagctgagcgagatcagcatggacaacagccccaacctggcctggcccctgatcaccgtggaggagctgaagaagctgctggagcagtggaacctggtgatcggcttcctgttcagcccccgctggtacttctactacctgggcaccggccccgaggccggcctgccctacggcgccatcagccgccagcgcctgaccaagtacaccatggccgacctggtgtacgccctgcgccacttcagcttcagcggctacctgaagctgaccgacaacgtgtacatcaagaacgccgacatcgtggaggcccccaccaaggtgaccttcggcgacgacaccgtgatcgaggtgcagggctacaagagcgtgaccgactacaagcactggccccagatcgcccagttcgcccccagcgccagcgccttcttcggcaagctgttcgccgccgagaccctgaaggccaccgaggagaccttcaagctgagctacggcatcaagaacctgagcgaccgcgtggtgttcgtgctgtgggcccacggcttcgagctgaccagcatgaagcactacgtgtacatcggcgaccccgcccagctgcccgccccccgcaccctgctgaccaagagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 115 - RNA, 1134 nt, synthetic construct
  misc_feature 1..1134: note = SARS-CoV-2_concat_2.2.2_DCL_opt3
  source 1..1134: mol_type = other RNA
atggccgccccaggctctgccagaagacctctgctgctgttgctgctgctgctgctgctgggcctgatgcactgcgcaagtgccgccctgtgtacttttctgctgaacaaagagatgtatctgaaactgagatctgacgtgctgctgccaatgtactcttttgtgagcgaggagacaggaacactgatcgtgaatagtgtgctgctgtttctggtgcccgtggtggacagctattactccctcctgatgcccatcctgacactgacccgagccctcaccagccacacagtgatgcctctgtctgcccccaccctggtgccacaggaacactacgtgcgggcctctgcactgtgggagatccagcaagtggtggatgccgatagcaagattgtccagctgtcagagatcagcatggacaattcccctaatctggcatggccactgatcacagtggaggagctgaaaaagctgctggagcagtggaacctggtgattggcttcctgttctcccccaggtggtatttctattatctggggaccggccctgaggccggcctgccctatggcgccattagcaggcagaggctgaccaaatacacaatggccgacctggtgtacgctctgagacatttttccttctccgggtacctgaagctgactgacaatgtgtacattaagaacgccgacatcgtggaggctcctaccaaggtcacatttggagacgacaccgtgatcgaggtgcagggctacaagagcgtgacagattacaaacactggccccagatcgcccagttcgcccccagcgcctccgccttctttgggaagctgttcgccgctgagacactgaaggccaccgaggagaccttcaaactgtcttacgggatcaagaatctgtcggatagggtggtgtttgtgctgtgggctcacgggttcgaactgacatccatgaagcactacgtgtacataggggaccccgcccagctgcccgcccccaggacgctcctgaccaaatcctcagactataccatcgtgctgcctgtgatcggggccattgtggtgggactgtgcctgatgggcatgggcgtctacaagatccggctgagatgccagagctccggctatcagcgcatctga

SEQ ID NO: 116 - RNA, 1479 nt, synthetic construct
  misc_feature 1..1479: note = SARS-CoV-2_concat_2.3_DCL_opt1
  source 1..1479: mol_type = other RNA
atggctgctcctggatctgctagaaggcctctgctgctgcttctgttgctcctgctgctgggcctgatgcattgtgcctctgcagcctctgccggctttcccttcaacaaatggggcaaagcccggctgtactacgacagcatgagctacaacctgatcgacagctacttcgtggtcaagcggcacaccttcagcaactaccagcacgaggaaccccagatcgcccagtttgccccttctgcctctgccttcttcggcatgagcagaatcggcatggacatcaaggacctccctaaagaaatcaccgtggccaccagcagaaccctgagctactacaagctgatcatcaacctggtgcagatggcccctatcagcgccatggtccgaatgtacatcttctttctgtgcaccttcctgctgaacaaagagatgtacctgaagctgcggagcgacgtgctgctgcctgcctctaagattctgggcctgcctacacagaccgtggatagctctcagggcagcgagtacgacatcagcagacagcggctgaccaagtacaccatggccgatctggtgtacgccctgagacacttcggcaccctgatgatcgagagattcgtgtccctggctatcgacgcctatcctctgaccaagcacctgacactgggcgtgtacgactacctggtgtctacccaagagttccggtacatgaacagccagggcctgctcatgtacagcttcgtgtccgaggaaaccggcacactgatcgtgaactctgtgctgctgttcctgggcctcgtggaagtggaaaaaggcgtgctgcctcagctggaacagccctacgtgttcatcaagcagtacatccggaagctgcacgacgagctgacaggccacatgctggacatgtactccgtgatgtatctgaacacactgaccctggccgtgccttacaacatgagagtgatccacttcggagccggcgagctgttcgagaacaagaccacactgcccgtgaacgtggcctttgagctgtgggccaagagaaagctgttcgccgccgagacactgaaggccaccgaagagacattcaagctgagctacggcatctgcagcgtgatcgacctcctgctcgacgacttcgtggaaatcatcaagagccaggacctgagccaggtggtgtccgacatcgattacgtgcccctgaagtccgccacctgtatcacccggtgcaacaagatcttcgtggacggcgtgcccttcgtggtgtccacaggctaccactttagagagctgggcgagctgaaagtgaccttctttcccgacctgaacggcgacgtggtggccatcgattataagcacagcagcgactacaccatcgtgctgccagtgatcggcgccatcgttgtgggactgtgtctgatgggcatgggagtgtacaagatccggctgagatgccagtccagcggctaccagagaatctga

SEQ ID NO: 117 - RNA, 1479 nt, synthetic construct
  misc_feature 1..1479: note = SARS-CoV-2_concat_2.3_DCL_opt2
  source 1..1479: mol_type = other RNA
atggccgcccccggcagcgcccgccgccccctgctgctgctgctgctgctgctgctgctgggcctgatgcactgcgccagcgccgccagcgccggcttccccttcaacaagtggggcaaggcccgcctgtactacgacagcatgagctacaacctgatcgacagctacttcgtggtgaagcgccacaccttcagcaactaccagcacgaggagccccagatcgcccagttcgcccccagcgccagcgccttcttcggcatgagccgcatcggcatggacatcaaggacctccccaaggagatcaccgtggccaccagccgcaccctgagctactacaagctgatcatcaacctggtgcagatggcccccatcagcgccatggtgcgcatgtacatcttcttcctgtgcaccttcctgctgaacaaggagatgtacctgaagctgcgcagcgacgtgctgctgcccgccagcaagatcctgggcctgcccacccagaccgtggacagcagccagggcagcgagtacgacatcagccgccagcgcctgaccaagtacaccatggccgacctggtgtacgccctgcgccacttcggcaccctgatgatcgagcgcttcgtgagcctggccatcgacgcctaccccctgaccaagcacctgaccctgggcgtgtacgactacctggtgagcacccaggagttccgctacatgaacagccagggcctgctgatgtacagcttcgtgagcgaggagaccggcaccctgatcgtgaacagcgtgctgctgttcctgggcctggtggaggtggagaagggcgtgctgccccagctggagcagccctacgtgttcatcaagcagtacatccgcaagctgcacgacgagctgaccggccacatgctggacatgtacagcgtgatgtacctgaacaccctgaccctggccgtgccctacaacatgcgcgtgatccacttcggcgccggcgagctgttcgagaacaagaccaccctgcccgtgaacgtggccttcgagctgtgggccaagcgcaagctgttcgccgccgagaccctgaaggccaccgaggagaccttcaagctgagctacggcatctgcagcgtgatcgacctcctgctggacgacttcgtggagatcatcaagagccaggacctgagccaggtggtgagcgacatcgactacgtgcccctgaagagcgccacctgtatcacccgctgcaacaagatcttcgtggacggcgtgcccttcgtggtgagcaccggctaccacttccgcgagctgggcgagctgaaggtgaccttcttccccgacctgaacggcgacgtggtggccatcgactacaagcacagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 118 - RNA, 1479 nt, synthetic construct
  misc_feature 1..1479: note = SARS-CoV-2_concat_2.3_DCL_opt3
  source 1..1479: mol_type = other RNA
atggccgcccctggctccgccagacggccactgcttctcttgctgctgctgctgctgctggggctgatgcactgtgcctccgccgccagcgccggcttcccctttaacaagtggggcaaggccaggctgtactatgattccatgagctacaacctgatcgactcctacttcgtggtgaaaagacacaccttcagcaattaccagcacgaagagcctcagatcgctcagttcgccccaagcgcctctgcattcttcgggatgtcccgcatcggcatggatatcaaggatctgcccaaggaaatcaccgtggccaccagtaggaccctgagctactataagctgatcatcaacctggtgcagatggcccctatcagcgctatggtgcggatgtacatttttttcctgtgcacctttctgctaaacaaggagatgtacctgaagctgagaagcgatgtgctgctgcctgccagcaaaattctgggacttcccacccagaccgtggatagcagccagggcagcgagtacgatatttccaggcagagactgacaaaatacaccatggccgacctggtgtacgctctgaggcacttcggaactctgatgatcgagaggtttgtgagcctggccatcgacgcctaccctctgaccaagcacctgaccctgggcgtgtatgactacctcgtgtccacacaggagtttcgctacatgaatagccaggggctgcttatgtactccttcgtgagcgaggagaccggaaccctgatcgtgaattccgtgctgctgttcctgggcctggtggaagtggagaagggagtgctgccccagctggagcagccctatgtgttcatcaaacagtatatccggaagctgcacgacgaactgaccggccacatgctggatatgtacagcgtcatgtacctcaacactctgacactggccgtgccctataatatgcgggttattcacttcggggctggggaactgttcgagaacaagaccacactgcccgtgaatgtggcattcgaactgtgggccaaaaggaaactgtttgccgctgagaccctgaaggccactgaagagaccttcaaactgagctacgggatctgctccgtgattgacctcctgctggacgacttcgtggaaatcatcaagagccaggacctgagccaggtggtgagcgatatcgactacgtgcctctcaagtccgccacatgtatcaccagatgcaataagatcttcgtggacggcgtgccgttcgtggtgtccaccggctatcattttcgggagctgggcgaactgaaggtgacatttttcccagatctcaatggggacgtggtggctatcgactacaagcactccagcgactacaccattgtgctgcccgtgatcggcgccatcgtggtcggcctgtgtctgatggggatgggggtgtataagatccggctgcggtgccagtcaagcggatatcagcgcatttga

SEQ ID NO: 119 - RNA, 2046 nt, synthetic construct
  misc_feature 1..2046: note = SARS-CoV-2_concat_2.4_DCL_opt1
  source 1..2046: mol_type = other RNA
atggctgctcctggatctgctagaaggcctctgctgctgcttctgttgctcctgctgctgggcctgatgcactgtgcctctgcagctctgacactgggcgtgtacgactacctggtgtctacccaagagttccggtacatgaacagccagggcctgctggcctctaagatcctgggactgcctacacagaccgtggatagctctcagggcagcgagtacgactgcagcgtgatcgatctgctgctcgacgacttcgtggaaatcatcaagagccaggacctgagccagtacatccggaagctgcacgatgagctgaccggccacatgctggacatgtacagcgtgatgctgacccctgtgtacagcttcctgcctggcgtgtactccgtgatctacctgtacctgaccttcaacctgatcgacagctacttcgtggtcaagcggcacaccttcagcaactaccagcacgaggaaagcgccggctttcccttcaacaaatggggcaaagcccggctgtactacgacagcatgagctactacctgaacacactgaccctggccgtgccttacaacatgagagtgatccacttcggcgctggccacatggaactgccaacaggtgttcacgccggcaccgacctggaaggcaatttctatggaccccaggtggtgtccgacatcgactacgtgccactgaagtccgccacctgtatcaccagatgcaaccaccacagcatcggcttcgattacgtgtacaaccccttcatgatcgacgtgcagcagtggggacctcagatcgcccagtttgccccttctgccagcgccttctttggcatgagccggatcggcatgaagctgttcgccgccgaaacactgaaggccaccgaggaaaccttcaagctgagctacggcatcggcgtgtccatctgcagcaccatgaccaacagacagttccaccagaagctgctgaagtctatcctgtgcaccttcctgctgaacaaagagatgtacctgaagctgcggagcgacgtgctgctgcctatgtactccttcgtgtccgaagagacaggcaccctgatcgtgaactctgtgctgctgttcctgggcacactgatgatcgagagattcgtgtccctggctatcgacgcctatcctctgaccaagcacgagctgttcgagaacaagaccacactgcccgtgaacgtggcctttgagctgtgggccaagagaggctgggactaccccaagtgcgatcgggccatgcctaacatgctgcggattatggccagcctgatcagccggcagagactgaccaagtacaccatggccgatctggtgtacgccctgagacacttcttcgcctacgccaaccggaacagattcctgtatattatcaagctgatcttcctgtggctgctggccgccatgcagcggaagcttgagaagatggctgaccaggctatgacccagatgtacaagcagctgatcatcaacctggtgcagatggcccctatcagcgccatggttcgaatgtacatcttcttcgacatcaaggacctcccgaaagaaatcaccgtggccaccagcagaaccctgtcctactacaagaaaatcttcgtggacggcgtgcccttcgtggtgtccacaggctaccactttagagagctgggcacaagcagcggcgacgccacaacagcctatgccaactccgtgttcaacatctgccaggccgtggtgttcgacgagatcagcatggccaccaactacgacctgagcgtggtcaatgcccggctgcgggaactgaaagtgaccttctttcccgacctgaacggcgacgtggtggcaatcgattacaagcacggactggtggaagtggaaaagggcgtgctgcctcagctggaacagccctacgtgttcatcaagtccagcgactacaccatcgtgctgccagtgatcggcgccatcgttgtgggactgtgtctgatgggcatgggagtgtacaagatccggctgagatgccagagcagcggctaccagagaatctga

SEQ ID NO: 120 - RNA, 2046 nt, synthetic construct
  misc_feature 1..2046: note = SARS-CoV-2_concat_2.4_DCL_opt2
  source 1..2046: mol_type = other RNA
atggccgcccccggcagcgcccgccgccccctgctgctgctgctgctgctgctgctgctgggcctgatgcactgcgccagcgccgccctgaccctgggcgtgtacgactacctggtgagcacccaggagttccgctacatgaacagccagggcctgctggccagcaagatcctgggcctgcccacccagaccgtggacagcagccagggcagcgagtacgactgcagcgtgatcgacctcctgctggacgacttcgtggagatcatcaagagccaggacctgagccagtacatccgcaagctgcacgacgagctgaccggccacatgctggacatgtacagcgtgatgctgacccccgtgtacagcttcctgcccggcgtgtacagcgtgatctacctgtacctgaccttcaacctgatcgacagctacttcgtggtgaagcgccacaccttcagcaactaccagcacgaggagagcgccggcttccccttcaacaagtggggcaaggcccgcctgtactacgacagcatgagctactacctgaacaccctgaccctggccgtgccctacaacatgcgcgtgatccacttcggcgccggccacatggagctgcccaccggcgtgcacgccggcaccgacctggagggcaacttctacggcccccaggtggtgagcgacatcgactacgtgcccctgaagagcgccacctgtatcacccgctgcaaccaccacagcatcggcttcgactacgtgtacaaccccttcatgatcgacgtgcagcagtggggcccccagatcgcccagttcgcccccagcgccagcgccttcttcggcatgagccgcatcggcatgaagctgttcgccgccgagaccctgaaggccaccgaggagaccttcaagctgagctacggcatcggcgtgagcatctgcagcaccatgaccaaccgccagttccaccagaagctgctgaagagcatcctgtgcaccttcctgctgaacaaggagatgtacctgaagctgcgcagcgacgtgctgctgcccatgtacagcttcgtgagcgaggagaccggcaccctgatcgtgaacagcgtgctgctgttcctgggcaccctgatgatcgagcgcttcgtgagcctggccatcgacgcctaccccctgaccaagcacgagctgttcgagaacaagaccaccctgcccgtgaacgtggccttcgagctgtgggccaagcgcggctgggactaccccaagtgcgaccgcgccatgcccaacatgctgcgcatcatggccagcctgatcagccgccagcgcctgaccaagtacaccatggccgacctggtgtacgccctgcgccacttcttcgcctacgccaaccgcaaccgcttcctgtacatcatcaagctgatcttcctgtggctgctggccgccatgcagcgcaagctggagaagatggccgaccaggccatgacccagatgtacaagcagctgatcatcaacctggtgcagatggcccccatcagcgccatggtgcgcatgtacatcttcttcgacatcaaggacctccccaaggagatcaccgtggccaccagccgcaccctgagctactacaagaagatcttcgtggacggcgtgcccttcgtggtgagcaccggctaccacttccgcgagctgggcaccagcagcggcgacgccaccaccgcctacgccaacagcgtgttcaacatctgccaggccgtggtgttcgacgagatcagcatggccaccaactacgacctgagcgtggtgaacgcccgcctgcgcgagctgaaggtgaccttcttccccgacctgaacggcgacgtggtggccatcgactacaagcacggcctggtggaggtggagaagggcgtgctgccccagctggagcagccctacgtgttcatcaagagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 121 - RNA, 2046 nt, synthetic construct
  misc_feature 1..2046: note = SARS-CoV-2_concat_2.4_DCL_opt3
  source 1..2046: mol_type = other RNA
atggccgccccaggctctgccaggcgccccctgctgctcctgctgctgctcctgctgctgggcctgatgcactgcgctagcgcagccctgacactgggcgtgtacgattacctggtgagcacacaggagttccggtacatgaatagccagggcctgctggccagcaaaattttggggctgcccacacagactgtggactcctcccagggctctgaatacgattgcagcgtgatcgacctcctgctggatgacttcgtggaaatcatcaagtctcaggatctgtcccagtacattcgcaaactgcacgatgagctgaccggacacatgctggatatgtacagcgtgatgctgacccctgtgtactccttcctgcctggcgtctacagcgtgatctacctgtatctgactttcaacctgatcgattcctacttcgtcgtgaagcgccacactttttccaactaccagcacgaggagagtgccggcttccccttcaacaaatggggcaaggccagactgtattacgattccatgtcttactacctgaatacactgacactggccgtgccctacaacatgagagtgatccactttggagccggccacatggagctgcctaccggggtgcacgccgggaccgacctggaagggaatttctatgggcctcaggtggtgtctgacattgactatgtgcccctgaaatccgccacttgcatcacccggtgcaaccatcactctatcggctttgactatgtgtacaacccattcatgattgacgtgcagcagtggggcccacagattgcccagttcgcccccagcgccagcgccttctttggcatgagcaggatcggcatgaagctctttgccgccgaaaccctgaaggcaaccgaggaaaccttcaaactgtcctacggtatcggagtgagcatctgctccacaatgaccaacaggcagtttcatcagaagctcctgaagtccatcctgtgcaccttcctgctgaataaggagatgtatctgaagctgagatccgacgtgttgctgcctatgtactcctttgtgagcgaggagacaggaactctgatcgtgaacagcgtgctgctgttcctgggaaccctgatgatcgagaggttcgtgagcctggccatcgacgcctaccctctgaccaaacacgaactgttcgagaataaaacaacactgccagtgaatgtggccttcgaactgtgggctaaaagaggctgggattatcctaaatgtgaccgggccatgcctaacatgctgaggattatggcttcactgatcagcaggcagagactgaccaagtacaccatggccgacctggtgtatgccctgaggcatttcttcgcctatgctaatagaaacaggttcctgtacatcattaagcttatctttttgtggctgctggccgccatgcagcggaagcttgagaaaatggctgatcaggccatgactcagatgtacaagcagctgatcatcaacctggtgcagatggcccccattagcgccatggtgcggatgtatatcttcttcgacatcaaggatctgccaaaagagatcacagtggccacatctcgtacgctgagctactacaagaagattttcgtggacggcgtgccattcgtggtgagcaccggctaccactttagagagctgggcaccagctctggagacgctaccaccgcatacgcaaacagcgtgtttaacatctgccaggccgtggtcttcgatgagatcagcatggctaccaactacgatctgagtgtggttaacgctcgcctgcgcgagcttaaggtcaccttcttcccagacctcaacggcgatgtggtcgccattgactacaaacacggcctggtggaggtggagaagggggtgctgccacagctggagcagccctacgtgttcattaagtctagcgattacacaatcgtgctgcccgttatcggagctattgtggtgggcctgtgcctgatgggcatgggagtgtacaagatcaggctgcgctgccagagctcagggtaccagaggatttga

SEQ ID NO: 122 - RNA, 1101 nt, synthetic construct
  misc_feature 1..1101: note = SARS-CoV-2_concat_2.4.1_DCL_opt1
  source 1..1101: mol_type = other RNA
atggctgctcctggatctgctagaaggcctctgctgctgcttctgttgctcctgctgctgggcctgatgcactgtgcctctgcagctctgacactgggcgtgtacgactacctggtgtctacccaagagttccggtacatgaacagccagggcctgctggcctctaagatcctgggactgcctacacagaccgtggatagctctcagggcagcgagtacgactgcagcgtgatcgatctgctgctcgacgacttcgtggaaatcatcaagagccaggacctgagccagtacatccggaagctgcacgatgagctgaccggccacatgctggacatgtacagcgtgatgctgacccctgtgtacagcttcctgcctggcgtgtactccgtgatctacctgtacctgaccttcaacctgatcgacagctacttcgtggtcaagcggcacaccttcagcaactaccagcacgaggaaagcgccggctttcccttcaacaaatggggcaaagcccggctgtactacgacagcatgagctactacctgaacacactgaccctggccgtgccttacaacatgagagtgatccacttcggcgctggccacatggaactgccaacaggtgttcacgccggcaccgacctggaaggcaatttctatggaccccaggtggtgtccgacatcgactacgtgccactgaagtccgccacctgtatcaccagatgcaaccaccacagcatcggcttcgattacgtgtacaaccccttcatgatcgacgtgcagcagtggggacctcagatcgcccagtttgccccttctgccagcgccttctttggcatgagccggatcggcatgaagctgttcgccgccgaaacactgaaggccaccgaggaaaccttcaagctgagctacggcatcggcgtgtccatctgcagcaccatgaccaacagacagttccaccagaagctgctgaagtccatcagcagcgactacaccatcgtgctgcctgtgatcggcgccatcgttgtgggactgtgtctgatgggcatgggagtgtacaagatccggctgagatgccagagcagcggctaccagagaatctga

SEQ ID NO: 123 - RNA, 1101 nt, synthetic construct
  misc_feature 1..1101: note = SARS-CoV-2_concat_2.4.1_DCL_opt2
  source 1..1101: mol_type = other RNA
atggccgcccccggcagcgcccgccgccccctgctgctgctgctgctgctgctgctgctgggcctgatgcactgcgccagcgccgccctgaccctgggcgtgtacgactacctggtgagcacccaggagttccgctacatgaacagccagggcctgctggccagcaagatcctgggcctgcccacccagaccgtggacagcagccagggcagcgagtacgactgcagcgtgatcgacctcctgctggacgacttcgtggagatcatcaagagccaggacctgagccagtacatccgcaagctgcacgacgagctgaccggccacatgctggacatgtacagcgtgatgctgacccccgtgtacagcttcctgcccggcgtgtacagcgtgatctacctgtacctgaccttcaacctgatcgacagctacttcgtggtgaagcgccacaccttcagcaactaccagcacgaggagagcgccggcttccccttcaacaagtggggcaaggcccgcctgtactacgacagcatgagctactacctgaacaccctgaccctggccgtgccctacaacatgcgcgtgatccacttcggcgccggccacatggagctgcccaccggcgtgcacgccggcaccgacctggagggcaacttctacggcccccaggtggtgagcgacatcgactacgtgcccctgaagagcgccacctgtatcacccgctgcaaccaccacagcatcggcttcgactacgtgtacaaccccttcatgatcgacgtgcagcagtggggcccccagatcgcccagttcgcccccagcgccagcgccttcttcggcatgagccgcatcggcatgaagctgttcgccgccgagaccctgaaggccaccgaggagaccttcaagctgagctacggcatcggcgtgagcatctgcagcaccatgaccaaccgccagttccaccagaagctgctgaagagcatcagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 124 - RNA, 1101 nt, synthetic construct
  misc_feature 1..1101: note = SARS-CoV-2_concat_2.4.1_DCL_opt3
  source 1..1101: mol_type = other RNA
atggcagcccctggctccgccagaagaccactgctgctcctgctgctgctgctcctgctgggcctgatgcactgcgcctccgctgccctgaccctgggcgtgtatgactacctggtgagcacccaggagttcagatacatgaactctcagggcctcctggcctccaagatcctgggcctgcccacccaaaccgtggactcatctcagggatccgagtacgattgctccgtgatcgacctcctgctggacgatttcgtggaaatcattaagagccaggatctgtctcagtatatcagaaagctgcatgacgagctgaccggccacatgctcgacatgtattccgtgatgctgacacccgtgtactctttcctgcccggcgtgtattctgtgatctatctctacctgaccttcaacctgatcgactcatacttcgtggtgaagagacataccttcagcaactatcagcacgaggagagcgctggcttcccctttaacaagtggggcaaggctagactgtactatgatagcatgtcttattacctgaacacactgaccctggctgtgccatacaacatgagggtgattcactttggcgctggacacatggagctgcctaccggagtgcacgccggcaccgatctggagggaaatttctatgggccccaggtggtgtccgacatcgactacgtgcccctgaagtcggccacatgtattactcgctgcaaccaccacagcattggctttgattatgtgtataaccccttcatgatcgacgtgcagcagtggggcccacagatcgctcagttcgccccttcagccagtgccttctttgggatgtccagaatcggaatgaagctgtttgccgccgagaccctgaaggccacagaagagacctttaagctgtcctacggaatcggcgtgagcatttgctccaccatgaccaatagacagttccaccagaaactgctgaagtctatttctagcgactataccattgtgctgcccgtgatcggggccatcgtggtgggcctgtgcctgatgggaatgggcgtgtacaagattaggctgaggtgtcagagcagcggctaccagcggatttaa

SEQ ID NO: 125 - RNA, 1155 nt, synthetic construct
  misc_feature 1..1155: note = SARS-CoV-2_concat_2.4.2_DCL_opt1
  source 1..1155: mol_type = other RNA
atggctgctcctggatctgctagaaggcctctgctgctgcttctgttgctcctgctgctgggcctgatgcactgtgcctctgcagccctgtgtaccttcctgctgaacaaagagatgtacctgaagctgcggagcgacgtgctgctgcccatgtacagctttgtgtccgaggaaaccggcacactgatcgtgaactctgtgctgctgttcctgggcaccctgatgatcgagagattcgtgtccctggctatcgacgcctatcctctgaccaagcacgagctgttcgagaacaagaccacactgcccgtgaacgtggcctttgagctgtgggccaagagaggctgggactaccccaagtgcgatcgggccatgcctaacatgctgcggattatggccagcctgatcagccggcagagactgaccaagtacaccatggccgatctggtgtacgccctgagacacttcttcgcctacgccaaccggaacagattcctgtatattatcaagctgatcttcctgtggctgctggccgccatgcagcggaagcttgagaagatggctgaccaggccatgacacagatgtacaagcagctgatcatcaacctggtgcagatggcccctatcagcgccatggtccgaatgtacatcttcttcgacatcaaggacctcccgaaagaaatcaccgtggccaccagcagaaccctgagctactacaagaaaatcttcgtggacggcgtgcccttcgtggtgtctaccggctaccactttagagagctgggcacaagcagcggcgacgccacaacagcctatgccaacagcgtgttcaacatctgccaggccgtggtgttcgacgagatcagcatggccaccaactacgacctgagcgtggtcaatgcccggctgcgggaactgaaagtgaccttctttcccgacctgaacggcgacgtggtggcaatcgattacaagcacggactggtggaagtggaaaagggcgtgctgcctcagctggaacagccctacgtgttcatcaagagcagcgactacaccatcgtgctgccagtgatcggcgccatcgttgtgggactgtgtctgatgggcatgggcgtgtacaagatccggctgagatgccagagcagcggctatcagagaatctga

SEQ ID NO: 126 - RNA, 1155 nt, synthetic construct
  misc_feature 1..1155: note = SARS-CoV-2_concat_2.4.2_DCL_opt2
  source 1..1155: mol_type = other RNA
atggccgcccccggcagcgcccgccgccccctgctgctgctgctgctgctgctgctgctgggcctgatgcactgcgccagcgccgccctgtgcaccttcctgctgaacaaggagatgtacctgaagctgcgcagcgacgtgctgctgcccatgtacagcttcgtgagcgaggagaccggcaccctgatcgtgaacagcgtgctgctgttcctgggcaccctgatgatcgagcgcttcgtgagcctggccatcgacgcctaccccctgaccaagcacgagctgttcgagaacaagaccaccctgcccgtgaacgtggccttcgagctgtgggccaagcgcggctgggactaccccaagtgcgaccgcgccatgcccaacatgctgcgcatcatggccagcctgatcagccgccagcgcctgaccaagtacaccatggccgacctggtgtacgccctgcgccacttcttcgcctacgccaaccgcaaccgcttcctgtacatcatcaagctgatcttcctgtggctgctggccgccatgcagcgcaagctggagaagatggccgaccaggccatgacccagatgtacaagcagctgatcatcaacctggtgcagatggcccccatcagcgccatggtgcgcatgtacatcttcttcgacatcaaggacctccccaaggagatcaccgtggccaccagccgcaccctgagctactacaagaagatcttcgtggacggcgtgcccttcgtggtgagcaccggctaccacttccgcgagctgggcaccagcagcggcgacgccaccaccgcctacgccaacagcgtgttcaacatctgccaggccgtggtgttcgacgagatcagcatggccaccaactacgacctgagcgtggtgaacgcccgcctgcgcgagctgaaggtgaccttcttccccgacctgaacggcgacgtggtggccatcgactacaagcacggcctggtggaggtggagaagggcgtgctgccccagctggagcagccctacgtgttcatcaagagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 127 - RNA, 1155 nt, synthetic construct
  misc_feature 1..1155: note = SARS-CoV-2_concat_2.4.2_DCL_opt3
  source 1..1155: mol_type = other RNA
atggccgccccaggctctgccaggcgccccctgctgctcctgctgctgctcctgctgctgggcctgatgcactgcgctagcgcagccctgtgtacattcctgctgaataaagagatgtatctgaagctgagatctgacgtcctgctgcccatgtacagcttcgtgtccgaggagacggggaccctgatcgtgaattctgtgctcctgttcctgggcaccctgatgattgagagattcgtgagcctggccatcgacgcctaccctctgaccaaacacgagctgttcgagaacaagaccaccctgcccgtgaacgtggcctttgagctgtgggctaagcgcggatgggactaccccaagtgcgaccgcgccatgcctaacatgctgagaattatggccagcttaatcagccggcagaggctgacaaaatacaccatggccgacctcgtgtacgccctgcggcacttcttcgcctacgcaaaccgcaaccgcttcctgtacatcatcaagctgatctttctgtggctgctggccgccatgcagcggaagctggaaaagatggccgaccaggccatgacccagatgtacaagcagctgatcatcaatctggtgcagatggccccaatctccgccatggtgcgaatgtacatttttttcgatattaaggacctccctaaggagattaccgtggccaccagccgtaccctgagctactacaaaaagatcttcgtggacggcgtgccctttgtggtgagcaccggctatcacttcagggaactgggtacctcctccggggatgcaactaccgcctacgccaattctgtgttcaacatttgccaggccgtggtcttcgatgagatcagcatggccaccaactacgatctgtctgtggtgaacgcccgcctgcgcgagctgaaagtgaccttcttccctgacctgaatggcgacgtggtggctattgactacaaacacggcctggtggaggtggaaaaaggagtgctgccccagctggagcagccatacgtgttcatcaaaagttctgattacaccatcgtgttgccagtgattggagctatcgtggtggggctgtgcctgatggggatgggcgtgtacaagattagactgaggtgccagagctctggctaccagagaatctga

SEQ ID NO: 128 - RNA, 840 nt, synthetic construct
  misc_feature 1..840: note = SARS-CoV-2_concat_2.5_DCL_opt1
  source 1..840: mol_type = other RNA
atggctgctcctggatctgctagaaggcctctgctgctgcttctgttgctcctgctgctgggcctgatgcactgtgcctctgcagccctgtgtaccttcctgctgaacaaagagatgtacctgaagctgcggagcgacgtgctgctgcccaagatctttgtggatggcgtgcccttcgtggtgtccaccggctaccactttagagagctgggctctgccggctttcccttcaacaaatggggcaaagcccggctgtactacgacagcatgagctacgacatcaaggacctccctaaagaaatcaccgtggccaccagcagaaccctgagctactacaagggcaccctgatgatcgagagattcgtgtccctggctatcgacgcctatcctctgaccaagcacgagctgttcgagaacaagaccacactgcccgtgaacgtggcctttgagctgtgggccaagagagccagcaagatcctgggactgcctacacagaccgtggatagctctcagggcagcgagtacgacaagctgtttgccgccgagacactgaaggccaccgaggaaaccttcaagctgtcctacggcatcatcagcagacagcggctgaccaagtacaccatggccgatctggtgtacgccctgcggcacttcatgtacagcttcgtgtccgaagagacaggcacactgatcgtgaactctgtgctgctgttcctgagcagcgactacaccatcgtgctgcctgtgatcggcgccatcgttgtgggactgtgtctgatgggcatgggcgtgtacaagatccggctgagatgccagagcagcggctatcagagaatctga

SEQ ID NO: 129 - RNA, 840 nt, synthetic construct
  misc_feature 1..840: note = SARS-CoV-2_concat_2.5_DCL_opt2
  source 1..840: mol_type = other RNA
atggccgcccccggcagcgcccgccgccccctgctgctgctgctgctgctgctgctgctgggcctgatgcactgcgccagcgccgccctgtgcaccttcctgctgaacaaggagatgtacctgaagctgcgcagcgacgtgctgctgcccaagatcttcgtggacggcgtgcccttcgtggtgagcaccggctaccacttccgcgagctgggcagcgccggcttccccttcaacaagtggggcaaggcccgcctgtactacgacagcatgagctacgacatcaaggacctccccaaggagatcaccgtggccaccagccgcaccctgagctactacaagggcaccctgatgatcgagcgcttcgtgagcctggccatcgacgcctaccccctgaccaagcacgagctgttcgagaacaagaccaccctgcccgtgaacgtggccttcgagctgtgggccaagcgcgccagcaagatcctgggcctgcccacccagaccgtggacagcagccagggcagcgagtacgacaagctgttcgccgccgagaccctgaaggccaccgaggagaccttcaagctgagctacggcatcatcagccgccagcgcctgaccaagtacaccatggccgacctggtgtacgccctgcgccacttcatgtacagcttcgtgagcgaggagaccggcaccctgatcgtgaacagcgtgctgctgttcctgagcagcgactacaccatcgtgctgcccgtgatcggcgccatcgtggtgggcctgtgcctgatgggcatgggcgtgtacaagatccgcctgcgctgccagagcagcggctaccagcgcatctaa

SEQ ID NO: 130 - RNA, 840 nt, synthetic construct
  misc_feature 1..840: note = SARS-CoV-2_concat_2.5_DCL_opt3
  source 1..840: mol_type = other RNA
atggccgccccaggcagcgccaggagaccactgctcctgctgctgctgctgctgctcctggggctgatgcactgcgcctctgccgctctgtgcaccttcctgctgaacaaggagatgtatctgaagctgaggtccgacgtgctgctgccaaaaattttcgtggacggcgtgccctttgtggtgtccaccggctaccacttcagggagctggggagcgccggcttcccctttaacaagtggggaaaagccagactgtactacgatagcatgagctacgacatcaaagatctgcctaaggagatcacagtggccacatccagaaccctgagttactacaagggaaccctcatgatcgagaggttcgtgagcctggccatcgacgcctaccctctgaccaagcacgagctgtttgaaaacaaaaccacactgcccgtcaacgtggctttcgagctgtgggccaagagggcctctaaaatcctgggcctgcctacccagaccgtggactcctcccagggctctgaatacgataaactgttcgccgccgagaccctgaaagccaccgaggagaccttcaaactgtcctacggaatcatcagccgccagaggctgaccaagtacaccatggctgacctcgtgtacgccctgcggcattttatgtacagcttcgtgagtgaggagaccggtacactgatcgtgaacagcgtgctgctgtttttgtctagcgactacaccatcgtgctgcccgtcatcggcgcgatcgtggtgggcctgtgcctgatgggcatgggtgtgtataagatccggctgaggtgtcagagctctggctaccagagaatctga

SEQ ID NO: 131 - RNA, 3816 nt, synthetic construct
  misc_feature 1..3816: note = glycoprotein_delta_opt1
  source 1..3816: mol_type = other RNA
atgttcgtgttcctggtgctgctgcctctggtgtccagccagtgtgtgaacctccggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctccgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctccagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcaggcggagagccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctcctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctcctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtcgtgaaccagaacgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctccaagaactggggaagtacgagcagtacatcaagtggccttggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaaaggcgtgaagctgcactacacctga

SEQ ID NO: 132 - RNA, 3816 nt, synthetic construct
  misc_feature 1..3816: note = glycoprotein_stab_opt1
  source 1..3816: mol_type = other RNA
atgttcgtgttcctggtgctgctgcctctggtgtccagccagtgtgtgaacctccggaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcggcgtgtacagcagcgccaacaactgcaccttcgagtacgtgtcccagcctttcctgatggacctggaaggcaagcagggcaacttcaagaacctccgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctccagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtacaggctgttccggaagtccaatctgaagcccttcgagcgggacatctccaccgagatctatcaggccggcagcaagccttgcaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagaccaacagcagaggctctgccagctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctcctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctcctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagccgggcctgctctgcagatcccatttccaatgcagatggcctaccggttcaacggcatcggagtgacccagaatgtgctgtacgagaaccagaagctgatcgccaaccagttcaacagcgccatcggcaagatccaggacagcctgagcagcacacctagcgctctgggcaagctgcagaacgtcgtgaaccagaatgcccaggcactgaacaccctggtcaagcagctgtcctccaacttcggcgccatctctagcgtgctgaacgatatcctgagcagactggaccctcctgaggccgaggtgcagatcgacagactgatcacaggcagactccagtctctgcagacctacgtgacccagcagctgatcagagccgccgagattagagccagcgctaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgtgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctccaagaactggggaagtacgagcagtacatcaagtggccttggtacatctggctgggctttatcgccggactgattgccatcgtgatggtcacaatcatgctgtgctgcatgaccagctgctgtagctgcctgaagggctgttgtagctgtggcagctgctgcaagttcgacgaggacgattctgagcccgtgctgaaaggcgtgaagctgcactacacctga

SEQ ID NO: 133 - RNA, 3816 nt, synthetic construct
  misc_feature 1..3816: note = glycoprotein_delta_opt2
  source 1..3816: mol_type = other RNA
atgttcgtgttcctggtgctgctgcccctggtgagcagccagtgcgtgaacctccgcacccgcacccagctgccccccgcctacaccaacagcttcacccgcggcgtgtactaccccgacaaggtgttccgcagcagcgtgctgcacagcacccaggacctgttcctgcccttcttcagcaacgtgacctggttccacgccatccacgtgagcggcaccaacggcaccaagcgcttcgacaaccccgtgctgcccttcaacgacggcgtgtacttcgccagcaccgagaagagcaacatcatccgcggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctccgcgagttcgtgttcaagaacatcgacggctacttcaagatctacagcaagcacacccccatcaacctggtgcgcgacctcccccagggcttcagcgccctggagcccctggtggacctccccatcggcatcaacatcacccgcttccagaccctgctggccctgcaccgcagctacctgacccccggcgacagcagcagcggctggaccgccggcgccgccgcctactacgtgggctacctccagccccgcaccttcctgctgaagtacaacgagaacggcaccatcaccgacgccgtggactgcgccctggaccccctgagcgagaccaagtgcaccctgaagagcttcaccgtggagaagggcatctaccagaccagcaacttccgcgtgcagcccaccgagagcatcgtgcgcttccccaacatcaccaacctgtgccccttcggcgaggtgttcaacgccacccgcttcgccagcgtgtacgcctggaaccgcaagcgcatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgcggcgacgaggtgcgccagatcgcccccggccagaccggcaagatcgccgactacaactacaagctgcccgacgacttcaccggctgcgtgatcgcctggaacagcaacaacctggacagcaaggtgggcggcaactacaactaccgctaccgcctgttccgcaagagcaacctgaagcccttcgagcgcgacatcagcaccgagatctaccaggccggcagcaagccctgcaacggcgtggagggcttcaactgctacttccccctgcagagctacggcttccagcccaccaacggcgtgggctaccagccctaccgcgtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggccgcgacatcgccgacaccaccgacgccgtgcgcgacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggcgcgtgtacagcaccggcagcaacgtgttccagacccgcgccggctgcctgatcggcgccgagcacgtgaacaacagctacgagtgcgacatccccatcggcgccggcatctgcgccagctaccagacccagaccaacagccgccgccgcgcccgcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctcctgctgcagtacggcagcttctgcacccagctgaaccgcgccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagcgcagcttcatcgaggacctcctgttcaacaaggtgaccctggccgacgccggcttcatcaagcagtacggcgactgcctgggcgacatcgccgcccgcgacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctaccgcttcaacggcatcggcgtgacccagaacgtgctgtacgagaaccagaagctgatcgccaaccagttcaacagcgccatcggcaagatccaggacagcctgagcagcaccgccagcgccctgggcaagctgcagaacgtggtgaaccagaacgcccaggccctgaacaccctggtgaagcagctgagcagcaacttcggcgccatcagcagcgtgctgaacgacatcctgagccgcctggacccccccgaggccgaggtgcagatcgaccgcctgatcaccggccgcctgcagagcctgcagacctacgtgacccagcagctgatccgcgccgccgagatccgcgccagcgccaacctggccgccaccaagatgagcgagtgcgtgctgggccagagcaagcgcgtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttcccccgcgagggcgtgttcgtgagcaacggcacccactggttcgtgacccagcgcaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgaccgcctgaacgaggtggccaagaacctgaacgagagcctgatcgacctccaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 134 - RNA, 3816 nt, synthetic construct
  misc_feature 1..3816: note = glycoprotein_stab_opt2
  source 1..3816: mol_type = other RNA
atgttcgtgttcctggtgctgctgcccctggtgagcagccagtgcgtgaacctccgcacccgcacccagctgccccccgcctacaccaacagcttcacccgcggcgtgtactaccccgacaaggtgttccgcagcagcgtgctgcacagcacccaggacctgttcctgcccttcttcagcaacgtgacctggttccacgccatccacgtgagcggcaccaacggcaccaagcgcttcgacaaccccgtgctgcccttcaacgacggcgtgtacttcgccagcaccgagaagagcaacatcatccgcggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctccgcgagttcgtgttcaagaacatcgacggctacttcaagatctacagcaagcacacccccatcaacctggtgcgcgacctcccccagggcttcagcgccctggagcccctggtggacctccccatcggcatcaacatcacccgcttccagaccctgctggccctgcaccgcagctacctgacccccggcgacagcagcagcggctggaccgccggcgccgccgcctactacgtgggctacctccagccccgcaccttcctgctgaagtacaacgagaacggcaccatcaccgacgccgtggactgcgccctggaccccctgagcgagaccaagtgcaccctgaagagcttcaccgtggagaagggcatctaccagaccagcaacttccgcgtgcagcccaccgagagcatcgtgcgcttccccaacatcaccaacctgtgccccttcggcgaggtgttcaacgccacccgcttcgccagcgtgtacgcctggaaccgcaagcgcatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgcggcgacgaggtgcgccagatcgcccccggccagaccggcaagatcgccgactacaactacaagctgcccgacgacttcaccggctgcgtgatcgcctggaacagcaacaacctggacagcaaggtgggcggcaactacaactaccgctaccgcctgttccgcaagagcaacctgaagcccttcgagcgcgacatcagcaccgagatctaccaggccggcagcaagccctgcaacggcgtggagggcttcaactgctacttccccctgcagagctacggcttccagcccaccaacggcgtgggctaccagccctaccgcgtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggccgcgacatcgccgacaccaccgacgccgtgcgcgacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggcgcgtgtacagcaccggcagcaacgtgttccagacccgcgccggctgcctgatcggcgccgagcacgtgaacaacagctacgagtgcgacatccccatcggcgccggcatctgcgccagctaccagacccagaccaacagccgcggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctcctgctgcagtacggcagcttctgcacccagctgaaccgcgccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagcgcagccccatcgaggacctcctgttcaacaaggtgaccctggccgacgccggcttcatcaagcagtacggcgactgcctgggcgacatcgccgcccgcgacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctaccgcttcaacggcatcggcgtgacccagaacgtgctgtacgagaaccagaagctgatcgccaaccagttcaacagcgccatcggcaagatccaggacagcctgagcagcacccccagcgccctgggcaagctgcagaacgtggtgaaccagaacgcccaggccctgaacaccctggtgaagcagctgagcagcaacttcggcgccatcagcagcgtgctgaacgacatcctgagccgcctggacccccccgaggccgaggtgcagatcgaccgcctgatcaccggccgcctgcagagcctgcagacctacgtgacccagcagctgatccgcgccgccgagatccgcgccagcgccaacctggccgccaccaagatgagcgagtgcgtgctgggccagagcaagcgcgtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttcccccgcgagggcgtgttcgtgagcaacggcacccactggttcgtgacccagcgcaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgaccgcctgaacgaggtggccaagaacctgaacgagagcctgatcgacctccaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 135 - protein, 18 aa, synthetic construct
  REGION 1..18: note = linker
KESGSVSSEQLAQFRSLD

SEQ ID NO: 136 - protein, 14 aa, synthetic construct
  REGION 1..14: note = linker2
EGKSSGSGSESKST